(19) United States
(12) Patent Application Publication
(10) Pub. No.: US 2010/0048597 A1
(43) Pub. Date: Feb. 25, 2010

(54) Organic Compounds and Their Uses

(75) Inventors: Rohan Eric John Beckwith, Cambridge, MA (US); Daniel Tim Curts, Cambridge, MA (US); Edmund Harrington, Cambridge, MA (US); Jurgen Hans-Hermann Hinrichs, Basel (CH); John Anthony Tallarico, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/520,755

(22) PCT Filed: Dec. 20, 2007
(86) PCT No.: PCT/US2007/088292

§ 371 Date: Jun. 22, 2009

Related U.S. Application Data
(60) Provisional application No. 60/871,471, filed on Dec. 22, 2006.

(51) Int. Cl. A61K 31/506; C07D 401/04; A61P 35/00

(52) U.S. Cl. 514/275; 544/297

(57) ABSTRACT

The present application describes organic compounds that are useful for the treatment, prevention and/or amelioration of diseases.

The present application claims priority to U.S. Ser. No. 60/871,471, filed Dec. 22, 2006, the entire specification of which is herein incorporated by reference.

### BACKGROUND

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. (Hardie, G. and Hanks, S. The Protein Kinase Facts Book, I and II, Academic Press, San Diego, Calif.: 1995). Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.). Sequence motifs have been identified that generally correspond to each of these kinase families (See, for example, Hanks, S. K., Hunter, T., FASEB J. 1995, 9, 576-596; Knighton et al., Science 1991, 253, 407-414; Hiles et al., Cell 1992, 70, 419-429; Kunz et al., Cell 1993, 73, 585-596; Garcia-Bustos et al., EMBO J. 1994, 13, 2352-2361).

Many diseases are associated with abnormal cellular responses triggered by the protein kinase-mediated events described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, viral diseases, and hormone-related diseases. Accordingly, there has been a substantial effort in medicinal chemistry to find protein kinase inhibitors that are effective as therapeutic agents.

The cyclin-dependent kinase (CDK) complexes are a class of kinases that are targets of interest. These complexes comprise at least a catalytic (the CDK itself) and a regulatory (cyclin) subunit. Some of the more important complexes for cell cycle regulation include cyclin A (CDK1—also known as cdc2, and CDK2), cyclin B1-B3 (CDK1) and cyclin D1-D3 (CDK2, CDK4, CDK5, CDK6), cyclin E (CDK2). Each of these complexes is involved in a particular phase of the cell cycle. Additionally, CDKs 7, 8, and 9 are implicated in the regulation of transcription.

The activity of CDKs is regulated post-translationally, by transitory associations with other proteins, and by alterations of their intracellular localization. Tumor development is closely associated with genetic alteration and deregulation of CDKs and their regulators, suggesting that inhibitors of CDKs may be useful anti-cancer therapeutics. Indeed, early results suggest that transformed and normal cells differ in their requirement for, e.g., cyclin A/CDK2 and that it may be possible to develop novel antineoplastic agents devoid of the general host toxicity observed with conventional cytotoxic and cytostatic drugs. While inhibition of cell cycle-related CDKs is clearly relevant in, e.g., oncology applications, inhibition of RNA polymerase-regulating CDKs may also be highly relevant in cancer indications.

The CDKs have been shown to participate in cell cycle progression and cellular transcription, and loss of growth control is linked to abnormal cell proliferation in disease (see e.g., Malumbres and Barbacid, Nat. Rev. Cancer 2001, 1:222). Increased activity or temporally abnormal activation of cyclin-dependent kinases has been shown to result in the development of human tumors (Sherr C. J., Science 1996, 274: 1672-1677). Indeed, human tumor development is commonly associated with alterations in either the CDK proteins themselves or their regulators (Cordon-Cardo C., Am. J. Pat 1/701. 1995; 147: 545-560; Karp J. E. and Broder S., Nat. Med. 1995; 1: 309-320; Hall M. et al., Adv. Cancer Res. 1996; 68: 67-108).

Naturally occurring protein inhibitors of CDKs such as p16 and p27 cause growth inhibition in vitro in lung cancer cell lines (Kamb A., Curr. Top. Microbiol. Immunol. 1998; 227: 139-148).

CDKs 7 and 9 play key roles in transcription initiation and elongation, respectively (see, e.g., Peterlin and Price. Cell 23: 297-305, 2006, Shapiro. J. Clin. Oncol. 24: 1770-83, 2006). Inhibition of CDK9 has been linked to direct induction of apoptosis in tumor cells of hematopoetic lineages through down-regulation of transcription of antiapoptotic proteins such as Mcl1 (Chao, S.-H. et al. J. Biol. Chem. 2000; 275:28345-28348; Chao, S.-H. et al. J. Biol. Chem. 2001; 276:31793-31799; Lam et. al. Genome Biology 2: 0041.1-11, 2001; Chen et al. Blood 2005; 106:2513; MacCallum et al. Cancer Res. 2005; 65:5399; and Alvi et al. Blood 2005; 105:4484). In solid tumor cells, transcriptional inhibition by downregulation of CDK9 activity synergizes with inhibition of cell cycle CDKs, for example CDK1 and 2, to induce apoptosis (Cai, D.-P., Cancer Res 2006, 66:9270. Inhibition of transcription through CDK9 or CDK7 may have selective killing activity in tumor cell types that are dependent on the transcription of mRNAs with short half lives, for example Cyclin D1 in Mantle Cell Lymphoma. Some transcription factors such as Myc and NF-kB selectively recruit CDK9 to their promoters, and tumors dependent on activation of these signaling pathways may be sensitive to CDK9 inhibition.

Small molecule CDK inhibitors may also be used in the treatment of cardiovascular disorders such as restenosis and atherosclerosis and other vascular disorders that are due to aberrant cell proliferation. Vascular smooth muscle proliferation and intimal hyperplasia following balloon angioplasty are inhibited by over-expression of the cyclin-dependent kinase inhibitor protein. Moreover, the purine CDK2 inhibitor CVT-313 (Ki=95 nM) resulted in greater than 80% inhibition of neointima formation in rats.

CDK inhibitors can be used to treat diseases caused by a variety of infectious agents, including fungi, protozoan parasites such as Plasmodium falciparum, and DNA and RNA viruses. For example, cyclin-dependent kinases are required for viral replication following infection by herpes simplex virus (HSV) (Schang L. M. et al., J. Virol. 1998; 72: 5626) and CDK homologs are known to play essential roles in yeast.

Inhibition of CDK9/cyclin T function was recently linked to prevention of HIV replication and the discovery of new CDK biology thus continues to open up new therapeutic indications for CDK inhibitors (Sausville, E. A. Trends Molec. Med; 2002, 8, S32-S37).

CDKs are important in neutrophil-mediated inflammation and CDK inhibitors promote the resolution of inflammation in animal models. (Rossi, A. G. et al, Nature Med. 2006, 12:1056). Thus CDK inhibitors, including CDK9 inhibitors, may act as anti-inflammatory agents.

Selective CDK inhibitors can be used to ameliorate the effects of various autoimmune disorders. The chronic inflammatory disease rheumatoid arthritis is characterized by synovial tissue hyperplasia; inhibition of synovial tissue proliferation should minimize inflammation and prevent joint destruction. In a rat model of arthritis, joint swelling was substantially inhibited by treatment with an adenovirus expressing a CDK inhibitor protein p 16. CDK inhibitors are effective against other disorders of cell proliferation including psoriasis (characterized by keratinocyte hyperproliferation), glomerulonephritis, chronic inflammation, and lupus.

Certain CDK inhibitors are useful as chemoprotective agents through their ability to inhibit cell cycle progression of normal untransformed cells (Chen, et al. J. Natl. Cancer Institute, 2000; 92: 1999-2008). Pre-treatment of a cancer patient with a CDK inhibitor prior to the use of cytotoxic agents can reduce the side effects commonly associated with chemotherapy. Normal proliferating tissues are protected from the cytotoxic effects by the action of the selective CDK inhibitor.

Accordingly, there is a great need to develop inhibitors of protein kinases, such as CDK1, CDK2, CDK3, CDK4, CDK5, CDK6, CDK7, CDK8 and CDK9, as well as combinations thereof.

### SUMMARY OF THE INVENTION

There remains a need for new treatments and therapies for protein kinase-associated disorders. There is also a need for compounds useful in the treatment or prevention or amelioration of one or more symptoms of cancer, inflammation, cardiac hypertrophy, and HIV. Furthermore, there is a need for methods for modulating the activity of protein kinases, such as CDK1, CDK2, CDK3, CDK4, CDK5, CDK6, CDK7, CDK8 and CDK9, and combinations thereof, using the compounds provided herein. In one aspect, the invention provides a compound of Formula I:

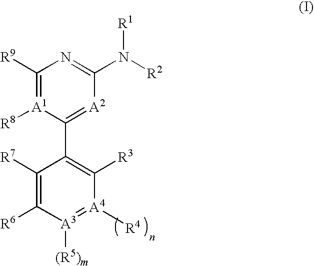

and pharmaceutically acceptable salts, enantiomers, stereoisomers, rotamers, tautomers, diastereomers, or racemates thereof.

In another aspect, the invention provides a compound of the Formula II:

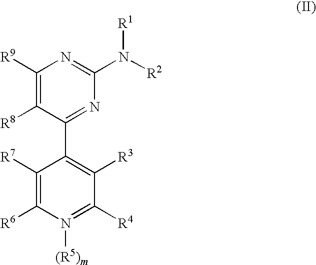

and pharmaceutically acceptable salts, enantiomers, stereoisomers, rotamers, tautomers, diastereomers, or racemates thereof.

In another aspect, the invention provides a compound of the Formula III:

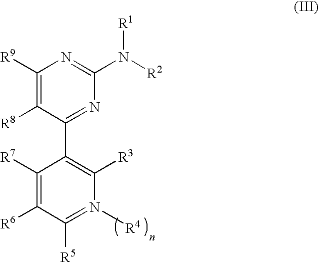

and pharmaceutically acceptable salts, enantiomers, stereoisomers, rotamers, tautomers, diastereomers, or racemates thereof.

In one embodiment, the compound of the invention is represented by a compound selected from Table A, Table B or Table C.

In another aspect, the invention provides a method of regulating, modulating, or inhibiting protein kinase activity which comprises contacting a protein kinase with a compound of the invention. In one embodiment, the protein kinase is selected from the group consisting of CDK1, CDK2, CDK3, CDK4, CDK5, CDK6, CDK7, CDK8 and CDK9, or any combination thereof. In another embodiment, the protein kinase is selected from the group consisting of CDK1, CDK2 and CDK9, or any combination thereof. In still another embodiment, the protein kinase is in a cell culture. In yet another embodiment, the protein kinase is in a mammal.

In another aspect, the invention provides a method of treating a protein kinase-associated disorder comprising administering to a subject in need thereof a pharmaceutically acceptable amount of a compound of the invention such that the protein kinase-associated disorder is treated. In one embodiment, the protein kinase is selected from the group consisting of CDK1, CDK2, CDK3, CDK4, CDK5, CDK6, CDK7, CDK8 and CDK9.

In one embodiment, the protein kinase-associated disorder is cancer. In still another embodiment, the cancer is selected from the group consisting of bladder, head and neck, breast, stomach, ovary, colon, lung, brain, larynx, lymphatic system, hematopoetic system, genitourinary tract, gastrointestinal, ovarian, prostate, gastric, bone, small-cell lung, glioma, colorectal and pancreatic cancer.

In one embodiment, the protein kinase-associated disorder is inflammation. In another embodiment, the inflammation is related to rheumatoid arthritis, lupus, type 1 diabetes, diabetic nephropathy, multiple sclerosis, glomerulonephritis, chronic inflammation, and organ transplant rejections.

In another embodiment, the protein kinase-associated disorder is a viral infection. In one embodiment, the viral infection is associated with the HIV virus, human papilloma virus, herpes virus, poxvirus virus, Epstein-Barr virus, Sindbis virus, or adenovirus.

In still another embodiment, the protein kinase-associated disorder is cardiac hypertrophy.

In another aspect, the invention provides a method of treating cancer comprising administering to a subject in need thereof a pharmaceutically acceptable amount of a compound of the invention such that the cancer is treated. In one embodiment, the cancer is selected from the group consisting of bladder, head and neck, breast, stomach, ovary, colon, lung, brain, larynx, lymphatic system, hematopoetic system, genitourinary tract, gastrointestinal, ovarian, prostate, gastric, bone, small-cell lung, glioma, colorectal and pancreatic cancer.

In another aspect, the invention provides a method of treating inflammation comprising administering to a subject in need thereof a pharmaceutically acceptable amount of a compound such that the inflammation is treated, wherein the compound is a compound of the invention. In one embodiment, the inflammation is related to rheumatoid arthritis, lupus, type 1 diabetes, diabetic nephropathy, multiple sclerosis, glomerulonephritis, chronic inflammation, and organ transplant rejections.

In another aspect, the invention provides a method of treating cardiac hypertrophy comprising administering to a subject in need thereof a pharmaceutically acceptable amount of a compound such that the cardiac hypertrophy is treated, wherein the compound is a compound of the invention.

In another aspect, the invention provides a method of treating a viral infection comprising administering to a subject in need thereof a pharmaceutically acceptable amount of a compound such that the viral infection is treated, wherein the compound is a compound of the invention. In one embodiment, the viral infection is associated with the HIV virus, human papilloma virus, herpes virus, poxvirus virus, Epstein-Barr virus, Sindbis virus, or adenovirus.

In one embodiment, the subject to be treated by the compounds of the invention is a mammal. In another embodiment, the mammal is a human.

In another aspect, the compounds of the invention is administered, simultaneously or sequentially, with an antiinflammatory, antiproliferative, chemotherapeutic agent, immunosuppressant, anti-cancer, cytotoxic agent or kinase inhibitor or salt thereof. In one embodiment, the compound, or salt thereof, is administered, simultaneously or sequentially, with one or more of a PTK inhibitor, cyclosporin A, CTLA4-Ig, antibodies selected from anti-ICAM-3, anti-IL-2 receptor, anti-CD45RB, anti-CD2, anti-CD3, anti-CD4, anti-CD80, anti-CD86, and monoclonal antibody OKT3, CVT-313, agents blocking the interaction between CD40 and gp39, fusion proteins constructed from CD40 and gp39, inhibitors of NF-kappa B function, non-steroidal antiinflammatory drugs, steroids, gold compounds, FK506, mycophenolate mofetil, cytotoxic drugs, TNF-α inhibitors, anti-TNF antibodies or soluble TNF receptor, rapamycin, leflunimide, cyclooxygenase-2 inhibitors, paclitaxel, cisplatin, carboplatin, doxorubicin, caminomycin, daunorubicin, aminopterin, methotrexate, methopterin, mitomycin C, ecteinascidin 743, porfiromycin, 5-fluorouracil, 6-mercaptopurine, gemcitabine, cytosine arabinoside, podophyllotoxin, etoposide, etoposide phosphate, teniposide, melphalan, vinblastine, vincristine, leurosidine, epothilone, vindesine, leurosine, or derivatives thereof.

In another aspect, the invention provides a packaged protein kinase-associated disorder treatment, comprising a protein kinase-modulating compound of the Formula I or Formula II, packaged with instructions for using an effective amount of the protein kinase-modulating compound to treat a protein kinase-associated disorder.

### DETAILED DESCRIPTION OF THE INVENTION

This invention is directed toward compounds, intermediates thereto and derivatives thereof, as well as pharmaceutical compositions containing the compounds for use in treatment of protein kinase-associated disorders. This invention is also directed to the compounds of the invention or compositions thereof as modulators of CDK1, CDK2, CDK3, CDK4, CDK5, CDK6, CDK7, CDK8 and CDK9, and combinations thereof. The present invention is also directed to methods of combination therapy for inhibiting protein kinase activity in cells, or for treating, preventing or ameliorating one or more symptoms of cancer, inflammation, cardiac hypertrophy, and viral disorders, (such as those associated with the HIV virus), using the compounds of the invention or pharmaceutical compositions, or kits thereof.

In one aspect, the invention provides compounds of the Formula I:

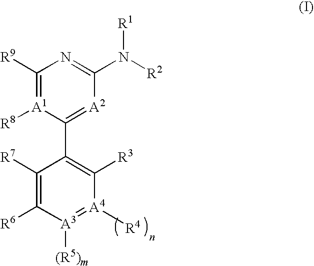

and pharmaceutically acceptable salts, enantiomers, stereoisomers, rotamers, tautomers, diastereomers, or racemates thereof;

wherein m is 0 or 1; n is 0 or 1; A¹, A², A³ and A⁴ are each, independently, C, C(H) or N; R¹, R², R³, R⁴, R⁵, R⁶, R⁷, R⁸ and R⁹ are each, independently, selected from the group consisting of hydrogen, halogen, substituted or unsubstituted amino, substituted or unsubstituted C₁₋₂₀-alkyl, substituted or unsubstituted C₁₋₂₀-alkoxy, substituted or unsubstituted aryl, and substituted or unsubstituted C₃₋₇-cycloalkyl; or R⁴ or R⁵ may independently be O⁻;

or R³ and R⁴ can also together form a six-membered ring of the following structure:

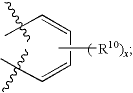

wherein x is 0, 1 or 2, and R¹⁰ is selected from the group consisting of hydrogen, halogen, substituted or unsubstituted amino, substituted or unsubstituted C₁₋₆-alkyl, substituted or unsubstituted C₁₋₆-alkoxy, substituted or unsubstituted aryl, and substituted or unsubstituted C₃₋₇-cycloalkyl;

or each of R⁷ and R⁸ can independently represent CH₂ or CH₂CH₂ groups that are bonded to one another, such that a six- or seven-membered ring is formed, or each of R³ and R⁸ can independently represent CH₂ or CH₂CH₂ groups that are bonded to one another, such that a six- or seven-membered ring is formed, wherein the six- or seven-membered rings may be independently substituted one or more times with hydrogen, halogen, substituted or unsubstituted amino, substituted or unsubstituted C₁₋₆-alkyl, substituted or unsubstituted C₁₋₆-alkoxy, substituted or unsubstituted aryl, or substituted or unsubstituted C₃₋₇-cycloalkyl.

In one embodiment of Formula I, R⁴ is hydrogen, (CH₂)₃₋₂₀CH₃ or (CH₂)₃₋₂₀NH₂, wherein the CH₂ groups can be independently interrupted one or more times with N(H), N(CH₃), O, or C(O). In another embodiment of Formula I, the C₁₋₂₀-alkyl and C₁₋₂₀-alkoxy groups are C₁₋₆-alkyl and C₁₋₆-alkoxy groups. In another embodiment of Formula I, at least one of R³, R⁷ and R⁸ is not H.

In another embodiment of Formula I, m is 0, A⁴ is C and A³ is N. Instill another embodiment of Formula I, A³ is C, A⁴ is N and n is 0. In yet another embodiment of Formula I, m and n are 0, A¹ is C and A and A⁴ are N. In another embodiment of Formula I, A¹ and A² are N. In still another embodiment of Formula I, n is 0, A¹ is C, and A³ and A⁴ are N.

In another embodiment of Formula I, R¹ is selected from the group consisting of hydrogen and C₁₋₆-alkyl; R² is selected from the group consisting of substituted-aryl and substituted-C₁₋₆-alkyl; R³ and R⁴ are each, independently, selected from the group consisting of hydrogen, halogen, C₁₋₆-alkyl, C₁₋₆-alkoxy, substituted or unsubstituted amino, substituted or unsubstituted aryl, and substituted or unsubstituted C₃₋₇-cycloalkyl;

R⁶ is selected from the group consisting of hydrogen, halogen and C₁₋₆-alkyl; and

R⁷, R⁸ and R⁹ are each, independently, selected from the group consisting of hydrogen and C₁₋₆-alkyl; or R³ and R⁴ can also together form a six-membered ring of the following structure:

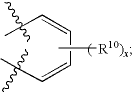

wherein x is 0, 1 or 2, and R¹⁰ is selected from the group consisting of hydrogen, halogen, substituted or unsubstituted amino, substituted or unsubstituted C₁₋₆-alkyl, substituted or unsubstituted C₁₋₆-alkoxy, and substituted or unsubstituted C₃₋₇-cycloalkyl;

or each of R⁷ and R⁸ can independently represent CH₂ or CH₂CH₂ groups that are bonded to one another, such that a six- or seven-membered ring is formed, or each of R³ and R⁸ can independently represent CH₂ or CH₂CH₂ groups that are bonded to one another, such that a six- or seven-membered ring is formed, wherein the six- or seven-membered rings may be independently substituted one or more times with hydrogen, halogen, substituted or unsubstituted amino, substituted or unsubstituted C₁₋₆-alkyl, substituted or unsubstituted C₁₋₆-alkoxy, substituted or unsubstituted aryl, or substituted or unsubstituted C₃₋₇-cycloalkyl.

In another embodiment of Formula I, R¹ is H. In yet another embodiment of Formula I, R² is aryl, which is independently substituted one or more times with substituted or unsubstituted alkyl, hydroxy, ether, thioether, amino, aryl, heterocycle, electron-withdrawing group or electron-withdrawing atom. In still another embodiment of Formula I, R² is benzothiazole, benzimidazole, benzoxazole, indazole or phenyl, all of which may be independently substituted one or more times with halogen, nitro, hydroxy, nitrile, substituted or unsubstituted amino, ether, ester, carboxylic acid, substituted or unsubstituted amide, substituted or unsubstituted sulfone, substituted or unsubstituted sulfonamide, substituted or unsubstituted phenyl or substituted or unsubstituted heterocycle.

In another embodiment of Formula I, at least one of R³, R⁷ and R⁸ is not H.

In another aspect of the invention, Formula I is represented by a compound of the Formula II:

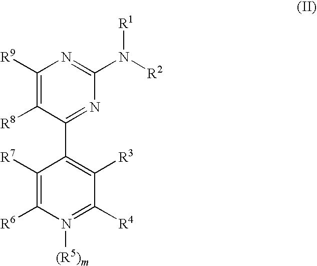

and pharmaceutically acceptable salts, enantiomers, stereoisomers, rotamers, tautomers, diastereomers, or racemates thereof;

wherein m is 0 or 1; R¹, R², R³, R⁴, R⁵, R⁶, R⁷, R⁸ and R⁹ are each, independently, selected from the group consisting of hydrogen, halogen, substituted or unsubstituted amino, substituted or unsubstituted C₁₋₂₀-alkyl, substituted or unsubstituted C₁₋₂₀-alkoxy, substituted or unsubstituted aryl, and substituted or unsubstituted C₃₋₇-cycloalkyl; or R⁵ may be O⁻;

or R³ and R⁴ can also together form a six-membered ring of the following structure:

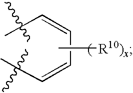

wherein x is 0, 1 or 2, and R¹⁰ is selected from the group consisting of hydrogen, halogen, substituted or unsubstituted amino, substituted or unsubstituted C₁₋₆-alkyl, substituted or unsubstituted C₁₋₆-alkoxy, and substituted or unsubstituted C₃₋₇-cycloalkyl;

or each of R⁷ and R⁸ can independently represent CH₂ or CH₂CH₂ groups that are bonded to one another, such that a six- or seven-membered ring is formed, or each of R³ and R⁸ can independently represent CH₂ or CH₂CH₂ groups that are bonded to one another, such that a six- or seven-membered ring is formed, wherein the six- or seven-membered rings may be independently substituted one or more times with hydrogen, halogen, substituted or unsubstituted amino, substituted or unsubstituted C₁₋₆-alkyl, substituted or unsubstituted C₁₋₆-alkoxy, substituted or unsubstituted aryl, or substituted or unsubstituted C₃₋₇-cycloalkyl.

In one embodiment of Formula II, R⁴ is hydrogen, (CH₂)₃₋₂₀CH₃ or (CH₂)₃₋₂₀NH₂, wherein the CH₂ groups can be independently interrupted one or more times with N(H), N(CH₃), O, or C(O). In another embodiment of Formula II, the C₁₋₂₀-alkyl and C₁₋₂₀-alkoxy groups are C₁₋₆-alkyl and C₁₋₆-alkoxy groups. In yet another embodiment of Formula II, at least one of R³, R⁷ and R⁸ is not H. In still another embodiment of Formula II, m is 0; R¹ is selected from the group consisting of hydrogen and C₁₋₆-alkyl; R² is selected from the group consisting of substituted-aryl and substituted-C₁₋₆-alkyl; R³ and R⁴ are each, independently, selected from the group consisting of hydrogen, halogen, C₁₋₆-alkyl, C₁₋₆-alkoxy, substituted or unsubstituted amino, substituted or unsubstituted aryl, and substituted or unsubstituted C₃₋₇-cycloalkyl; R⁶ is selected from the group consisting of hydrogen, halogen and C₁₋₆-alkyl; and R⁷, R⁸ and R⁹ are each, independently, selected from the group consisting of hydrogen, halogen, C₁₋₆-alkyl, C₁₋₆-alkoxy, and substituted or unsubstituted amino.

In another embodiment of Formula II, R² is aryl, which is independently substituted one or more times with substituted or unsubstituted alkyl, hydroxy, ether, thioether, amino, substituted or unsubstituted aryl, heterocycle, electron-withdrawing group or electron-withdrawing atom.

In another embodiment of Formula II, R² is benzothiazole, benzimidazole, benzoxazole, indazole or phenyl, all of which may be independently substituted one or more times with halogen, nitro, hydroxy, nitrile, substituted or unsubstituted amino, ether, ester, carboxylic acid, substituted or unsubstituted amide, substituted or unsubstituted sulfone, substituted or unsubstituted sulfonamide, substituted or unsubstituted phenyl or substituted or unsubstituted heterocycle. In still another embodiment of Formula II, R³ is selected from the group consisting of C₁₋₆-alkyl, C₁₋₆-alkoxy, and substituted or unsubstituted amino.

In another embodiment of Formula II, m is 0; R¹ is selected from the group consisting of hydrogen and C₁₋₆-alkyl; R² is benzothiazole, benzimidazole, benzoxazole, indazole or phenyl, all of which may be independently substituted one or more times with nitrile, halogen, nitro, hydroxy, substituted or unsubstituted amino, ether, ester, carboxylic acid, substituted or unsubstituted amide, substituted or unsubstituted sulfone, substituted or unsubstituted sulfonamide, substituted or unsubstituted phenyl or substituted or unsubstituted heterocycle; R³ and R⁴ are each, independently, selected from the group consisting of hydrogen, halogen, C₁₋₆-alkyl, C₁₋₆-alkoxy, substituted or unsubstituted amino, substituted or unsubstituted aryl, and substituted or unsubstituted C₃₋₇-cycloalkyl; R⁶ and R⁹ are H; and R⁷, and R⁸ are each, independently, selected from the group consisting of hydrogen, C₁₋₆-alkyl and C₁₋₆-alkoxy.

In another embodiment of Formula II, R² is selected from the group consisting of aryl substituted halogen, nitro, hydroxyl, nitrile, ether, ester, substituted or unsubstituted phenyl, substituted or unsubstituted heterocycle, SO₂(R¹¹) or N(R¹²)R¹³, and (CH₂)₁₋₆N(R¹²)R¹³; R³ is selected from the group consisting of C₁₋₆-alkyl, C₁₋₆-alkoxy and N(R¹²)R¹³; and R⁴ is selected from the group consisting of hydrogen and N(R¹²)R¹³; wherein R¹¹ is selected from the group consisting of H, C₁₋₆-alkyl and NH₂;

wherein R¹² and R¹³ are each, independently, selected from the group consisting of H and (C₁₋₆alkyl)₀₋₁G, wherein G is selected from the group consisting of H, COOH, NH₂, N(H)C(O)C₁₋₆alkyl, N(C₁₋₆alkyl)C(O)C₁₋₆alkyl, N(H)C₁₋₆alkyl, OH, OC(O)C₁₋₆alkyl, C₃₋₇-cycloalkyl, phenyl, substituted phenyl, C(O)OC₁-C₆-alkyl, C(O)C₁₋₆alkyl-COOH, C(O)C₁-C₄-alkyl, C(O)-aryl, morpholino, imidazole, pyrrolidin-2-one, substituted or unsubstituted heterocycle, pyrazole, pyridine, oxazole, thiazole, isoxazole, triazole, tetrazole, pyrimidine, pyridazine, pyrazine, piperazine and piperidine.

In another embodiment of Formula II, R² is substituted or unsubstituted imidazole, oxazole or triazole. In yet another embodiment of Formula II, m is 0; R¹ is hydrogen; R² is selected from the group consisting of aryl substituted halogen, nitro, hydroxyl, nitrile, ether, ester, substituted or unsubstituted phenyl, substituted or unsubstituted heterocycle, SO₂(R¹¹) or N(R¹²)R¹³, and (CH₂)₁₋₆N(R¹²)R¹³; R³ and R⁴ are each, independently, selected from the group consisting of hydrogen, halogen, C₁₋₆-alkyl, C₁₋₆-alkoxy, substituted or unsubstituted amino, substituted or unsubstituted aryl, and substituted or unsubstituted C₃₋₇-cycloalkyl; R⁶ is selected from the group consisting of hydrogen, chloro, and CH₃; and R⁷ and R⁸ are each, independently, selected from the group consisting of hydrogen, halogen, substituted or unsubstituted amino, C₁₋₆-alkyl and C₁₋₆-alkoxy; R⁹ is hydrogen; wherein R¹¹ is selected from the group consisting of H, C₁₋₆-alkyl and NH₂; wherein R¹² and R¹³ are each, independently, selected from the group consisting of H and (C₁₋₆alkyl)₀₋₁G, wherein G is selected from the group consisting of H, COOH, NH₂, N(H)C(O)C₁₋₆alkyl, N(C₁₋₆alkyl)C(O)C₁₋₆alkyl, N(H)C₁₋₆alkyl, OH, OC(O)C₁₋₆alkyl, C₃₋₇-cycloalkyl, phenyl, substituted phenyl, C(O)OC₁-C₆-alkyl, C(O)C₁₋₆alkyl-COOH, C(O)C₁-C₄-alkyl, C(O)-aryl, morpholino, imidazole and pyrrolidin-2-one.

In another embodiment of Formula II, m is 0; R¹ is hydrogen; R² is selected from the group consisting of aryl substituted with halogen, nitro, hydroxyl, nitrile, ether, ester, substituted or unsubstituted phenyl, substituted or unsubstituted heterocycle, SO₂(R¹¹), N(R¹²)R¹³, or (CH₂)₁₋₆N(R¹²)R¹³; R³ is selected from the group consisting of halogen, CH₃, CH₂CH₃, OCH₃ and substituted or unsubstituted amine; R⁴ is selected from the group consisting of hydrogen, halogen, CH₃, OCH₃, N(H)(CH₂)₃NH₂, N(H)(CH₂)₃N(H)C(O)CH₃, N(CH₃)(CH₂)₃N(H)CH₃, N(CH₃)(CH₂)₃N(CH₃)C(O)CH₃, N(H)(CH₂)₃OH, N(H)(CH₂)₃OC(O)CH₃, N(H)(CH₂)₃N(H)-Ph-CH₂NH₂, N(H)(CH₂)₃N(H)-Ph-CH₂N(H)C(O)CH₃, N(H)(CH₂)₃CO₂H, N(H)CH₂Ph, N(H)(CH₂)₃OCH₃, N(H)(CH₂)₃-pyrrolidin-2-one, N(H)(CH₂)₃—N-morpholino, N(H)(CH₂)₃-imidazole, N(H)(CH₂)₃—N(H)CH₃; R⁶ is selected from the group consisting of hydrogen, chloro, and CH₃; and R⁷ and R⁸ are each, independently, selected from the group consisting of hydrogen, halogen, substituted or unsubstituted amino, C₁₋₆-alkyl and C₁₋₆-alkoxy; R⁹ is hydrogen; wherein R¹¹ is selected from the group consisting of H, C₁₋₆-alkyl and NH₂; wherein R¹² and R¹³ are each, independently, selected from the group consisting of H and (C₁₋₆alkyl)₀₋₁G, wherein G is selected from the group consisting of H, COOH, NH₂, N(H)C(O)C₁₋₆alkyl, N(C₁₋₆alkyl)C(O)C₁₋₆alkyl, N(H)C₁₋₆alkyl, OH, OC(O)C₁₋₆alkyl, C₃₋₇-cycloalkyl, phenyl, substituted phenyl, C(O)OC₁-C₆-alkyl, C(O)C₁₋₆alkyl-COOH, C(O)C₁-C₄-alkyl, C(O)-aryl, morpholino, imidazole and pyrrolidin-2-one.

In another embodiment of Formula II, R³ is selected from the group consisting of CH₃, CH₂CH₃, OCH₃, OCH₂CH₃, O(CH₂)₃NH₂, O(CH₂)₃N(H)C(O)CH₃, N(H)(CH₂)₃OH, N(H)(CH₂)₃OCH₃, N(H)(CH₂)₃OC(O)CH₃, F, Cl, Br, NH₂, N(H)C(O)CH₃, N(H)(CH₂)₃CH₃, N(H)(CH₂)₂NH₂, N(H)(CH₂)₂N(H)C(O)CH₃, N(H)(CH₂)₂N(H)CH₃, N(H)(CH₂)₂N(CH₃)₂, N(H)CH₂C(CH₃)₂CH₂N(H)C(O)CH₃, N(H)(CH₂)₂-imidazole, N(H)(CH₂)₂-pyrazole, N(H)(CH₂)₃-imidazole, N(H)(CH₂)₃-pyrazole, N(H)(CH₂)₃-imidazole-CH₃, N(H)(CH₂)₃-pyrazole-CH₃, N(H)(CH₂)₃-imidazole-(CH₃)₂, N(H)(CH₂)₃-pyrazole-(CH₃)₂, N(H)(CH₂)₃-morpholino, N(H)(CH₂)₃-piperidine, N(H)(CH₂)₃-piperazine, N(H)(CH₂)₃-lactam, N(H)(CH₂)₃-pyrrolidin-2-one, N(H)(CH₂)₃NH₂, N(H)(CH₂)₃N(H)C(O)CH₃, N(H)(CH₂)₃N(H)C(O)C(CH₃)₃, N(H)(CH₂)₃N(H)C(O)CH₂C(CH₃)₃, N(H)CH₂C(CH₃)₂CH₂NH₂, N(H)(CH₂)₃N(H)CH₃, N(H)(CH₂)₃N(CH₃)₂, N(H)(CH₂)₃N(H)C(H)(CH₃)₂, N(H)(CH₂)₃N(H)S(O)₂CH₃, N(H)(CH₂)₄NH₂, N(H)(CH₂)₄N(H)C(O)CH₃, and N(H)-Ph-S(O)₂CH₃

In another aspect, Formula I is represented by a compound of the Formula III:

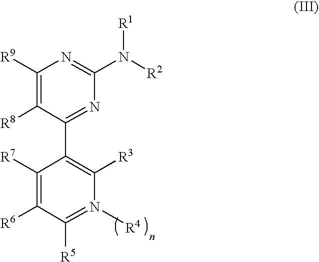

and pharmaceutically acceptable salts, enantiomers, stereoisomers, rotamers, tautomers, diastereomers, or racemates thereof;

wherein n is 0 or 1, R¹, R², R³, R⁴, R⁵, R⁶, R⁷, R⁸ and R⁹ are each, independently, selected from the group consisting of hydrogen, halogen, substituted or unsubstituted amino, substituted or unsubstituted C₁₋₂₀-alkyl, substituted or unsubstituted C₁₋₂₀-alkoxy, substituted or unsubstituted aryl, and substituted or unsubstituted C₃₋₇-cycloalkyl; or R⁴ may be O⁻;

or R³ and R⁴ can also together form a six-membered ring of the following structure:

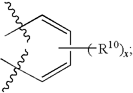

wherein x is 0, 1 or 2, and R¹⁰ is selected from the group consisting of hydrogen, halogen, substituted or unsubstituted amino, substituted or unsubstituted C₁₋₆-alkyl, substituted or unsubstituted C₁₋₆-alkoxy, and substituted or unsubstituted C₃₋₇-cycloalkyl;

or each of R⁷ and R⁸ can independently represent CH₂ or CH₂CH₂ groups that are bonded to one another, such that a six- or seven-membered ring is formed, or each of R³ and R⁸ can independently represent CH₂ or CH₂CH₂ groups that are bonded to one another, such that a six- or seven-membered ring is formed, wherein the six- or seven-membered rings may be independently substituted one or more times with hydrogen, halogen, substituted or unsubstituted amino, substituted or unsubstituted C₁₋₆-alkyl, substituted or unsubstituted C₁₋₆-alkoxy, substituted or unsubstituted aryl, or substituted or unsubstituted C₃₋₇-cycloalkyl.

In one embodiment of Formula III, R³ is halogen, (CH₂)₃₋₂₀CH₃ or (CH₂)₃₋₂₀NH₂, wherein the CH₂ groups can be independently interrupted one or more times with N(H), N(CH₃), O, or C(O). In another embodiment of Formula III, the C₁₋₂₀-alkyl and C₁₋₂₀-alkoxy groups are C₁₋₆-alkyl and C₁₋₆-alkoxy groups. In another embodiment of Formula III, at least one of R³, R⁷ and R⁸ is not H. In still another embodiment of Formula III, R¹ is H. In another embodiment of Formula III, n is 0, and R⁶, R⁷ and R⁹ are hydrogen.

In another embodiment of Formula III, R² is benzothiazole, benzimidazole, benzoxazole, indazole or phenyl, all of which may be independently substituted one or more times with halogen, nitro, hydroxy, nitrile, substituted or unsubstituted amino, ether, ester, carboxylic acid, substituted or unsubstituted amide, substituted or unsubstituted sulfone, substituted or unsubstituted sulfonamide, substituted or unsubstituted phenyl or substituted or unsubstituted heterocycle.

In another embodiment of Formula III, n is 0; R¹ is selected from the group consisting of hydrogen and C₁₋₆-alkyl; R² is selected from the group consisting of aryl substituted with halogen, nitro, hydroxyl, nitrile, ether, ester, substituted or unsubstituted phenyl, substituted or unsubstituted heterocycle, SO₂(R¹¹), N(R¹²)R¹³, or (CH₂)₁₋₆N(R¹²)R¹³;

R³ is selected from the group consisting of hydrogen, halogen, C₁₋₆-alkyl, C₁₋₆-alkoxy, substituted or unsubstituted amino, substituted or unsubstituted aryl, and substituted or unsubstituted C₃₋₇-cycloalkyl;

R⁷ and R⁸ are hydrogen, halogen, alkyl, alkoxy, or substituted or unsubstituted amino; R⁶ and R⁹ are H, wherein R¹¹ is selected from the group consisting of H, C₁₋₆-alkyl and NH₂; wherein R¹² and R¹³ are each, independently, selected from the group consisting of H and (C₁₋₆alkyl)₀₋₁G, wherein G is selected from the group consisting of H, COOH, NH₂, N(H)C(O)C₁₋₆alkyl, N(C₁₋₆alkyl)C(O)C₁₋₆alkyl, N(H)C₁₋₆alkyl, OH, OC(O)C₁₋₆alkyl, C₃₋₇-cycloalkyl, phenyl, substituted phenyl, C(O)OC₁-C₆-alkyl, C(O)C₁₋₆alkyl-COOH, C(O)C₁-C₄-alkyl, C(O)-aryl, morpholino, imidazole, pyrrolidin-2-one substituted or unsubstituted heterocycle, pyrazole, pyridine, oxazole, thiazole, isoxazole, triazole, tetrazole, pyrimidine, pyridazine, pyrazine, piperazine and piperidine.

In another embodiment of Formula III, R³ is selected from the group consisting of halogen, C₁₋₆-alkyl, C₁₋₆-alkoxy, and N(R¹²)R¹³; wherein R¹² and R¹³ are each, independently, selected from the group consisting of H and (C₁₋₆alkyl)₀₋₁G, wherein G is selected from the group consisting of H, COOH, NH₂, N(H)C(O)C₁₋₆alkyl, N(C₁₋₆alkyl)C(O)C₁₋₆alkyl, N(H)C₁₋₆alkyl, OH, OC(O)C₁₋₆alkyl, C₃₋₇-cycloalkyl, phenyl, substituted phenyl, C(O)OC₁-C₆-alkyl, C(O)C₁₋₆alkyl-COOH, C(O)C₁-C₄-alkyl, C(O)-aryl, morpholino, imidazole, pyrrolidin-2-one, substituted or unsubstituted heterocycle, pyrazole, pyridine, oxazole, thiazole, isoxazole, triazole, tetrazole, pyrimidine, pyridazine, pyrazine, piperazine and piperidine.

In another embodiment of Formula III, R¹ is hydrogen; and R² is hydrogen, CH₃, C₃₋₇-cyclohexyl, indazole, benzothiazole, benzimidazole, benzoxazole or phenyl, wherein the cyclohexyl, indazole, benzothiazole, benzimidazole, benzoxazole or phenyl groups are independently substituted one or more times with chloro, S(O)₂CH₃, C(O)NH₂, C(O)O-t-buyl, piperidine, piperidine substituted by t-butyl, oxazole, N(H)C(O)CH₃, 1,2,4-triazole substituted by methyl, SO₂NH₂, cyano, C(O)OEt, phenyl, S(O)₂N(H)CH₃, S(O)₂N(Et)₂, S(O)₂N(H)Et, S(O)₂N-imidazole, S(O)₂N(H)butyl, pyrrolidine, S(O)₂N(H)cyclohexyl, COOH, C(O)N(H)(CH₂)₂N(H)C(O)CH₃, C(O)N(H)(CH₂)₂N(CH₃)₂, C(O)N(H)Et, NO₂ or S(O)₂N(H)(CH₂)₂OH.

In another embodiment of Formula III, R³ is selected from the group consisting of CH₃, CH₂CH₃, OCH₃, OCH₂CH₃, O(CH₂)₃NH₂, O(CH₂)₃N(H)C(O)CH₃, N(H)(CH₂)₃OH, N(H)(CH₂)₃OCH₃, N(H)(CH₂)₃OC(O)CH₃, F, Cl, Br, NH₂, N(H)C(O)CH₃, N(H)(CH₂)₃CH₃, N(H)(CH₂)₂NH₂, N(H)(CH₂)₂N(H)C(O)CH₃, N(H)(CH₂)₂N(H)CH₃, N(H)(CH₂)₂N(CH₃)₂, N(H)CH₂C(CH₃)₂CH₂N(H)C(O)CH₃, N(H)(CH₂)₂-imidazole, N(H)(CH₂)₂-pyrazole, N(H)(CH₂)₃-imidazole, N(H)(CH₂)₃-pyrazole, N(H)(CH₂)₃-imidazole-CH₃, N(H)(CH₂)₃-pyrazole-CH₃, N(H)(CH₂)₃-imidazole-(CH₃)₂, N(H)(CH₂)₃-pyrazole-(CH₃)₂, N(H)(CH₂)₃-morpholino, N(H)(CH₂)₃-piperidine, N(H)(CH₂)₃-piperazine, N(H)(CH₂)₃-lactam, N(H)(CH₂)₃-pyrrolidin-2-one, N(H)(CH₂)₃NH₂, N(H)(CH₂)₃N(H)C(O)CH₃, N(H)(CH₂)₃N(H)C(O)C(CH₃)₃, N(H)(CH₂)₃N(H)C(O)CH₂C(CH₃)₃, N(H)CH₂C(CH₃)₂CH₂NH₂, N(H)(CH₂)₃N(H)CH₃, N(H)(CH₂)₃N(CH₃)₂, N(H)(CH₂)₃N(H)C(H)(CH₃)₂, N(H)(CH₂)₃N(H)S(O)₂CH₃, N(H)(CH₂)₄NH₂, N(H)(CH₂)₄N(H)C(O)CH₃, and N(H)-Ph-S(O)₂CH₃.

In yet another embodiment of Formula III, R⁵ is selected from the group consisting of fluorine, hydrogen and OCH₃. In still another embodiment of Formula III, R⁸ is selected from the group consisting of hydrogen, fluorine, OCH₃ and CH₃.

Preferred embodiments of Formula I and Formula II (including pharmaceutically acceptable salts thereof, as well as enantiomers, stereoisomers, rotamers, tautomers, diastereomers, atropisomers or racemates thereof) are shown below in Table A, Table B, and Table C, and are also considered to be “compounds of the invention.” The compounds of the invention are also referred to herein as “protein kinase inhibitors,” as well as “CDK inhibitors.”

TABLE A

| | Compound | |
|---|---|---|
| Compound name | no. | Structure |
| [4-(3-Amino-pyridin-4-yl)- pyrimidin-2-yl]-(3-chloro-phenyl)- amine | 1 | 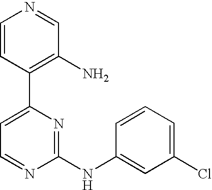 |
| [4-(2-Chloro-3-methyl-pyridin-4- yl)-pyrimidin-2-yl]-(3-chloro- phenyl)-amine | 2 | 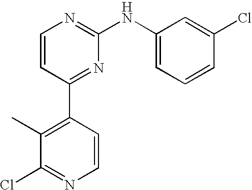 |
| N-{4-[2-(3-Chloro-phenylamino)- pyrimidin-4-yl]-3-methyl-pyridin- 2-yl}-N′-methyl-ethane-1,2- diamine | 3 | 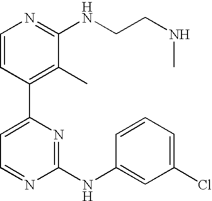 |
| N-{4-[2-(3-Chloro-phenylamino)- pyrimidin-4-yl]-3-methyl-pyridin- 2-yl}-propane-1,3-diamine | 4 | 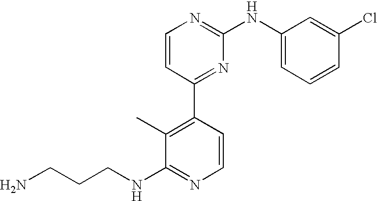 |
| N-{4-[2-(3-Chloro-phenylamino)- pyrimidin-4-yl]-3-ethyl-pyridin-2- yl}-propane-1,3-diamine | 5 | 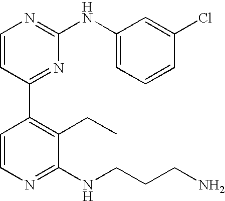 |
| N-(3-{4-[2-(3-Chloro- phenylamino)-pyrimidin-4-yl]-3- methyl-pyridin-2-ylamino}- propyl)-acetamide | 6 | 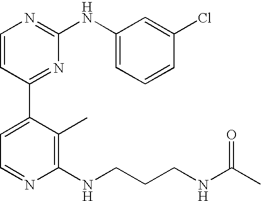 |
| N-(3-{4-[2-(3-Chloro- phenylamino)-pyrimidin-4-yl]-3- ethyl-pyridin-2-ylamino}-propyl)- acetamide | 7 | 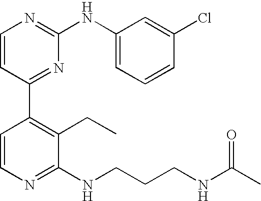 |
| N-(4-{2-[(3-Chloro-phenyl)- methyl-amino]-pyrimidin-4-yl}-3- methyl-pyridin-2-yl)-propane-1,3- diamine | 8 | 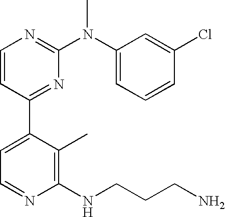 |
| N-{4-[2-(3-Chloro-phenylamino)- pyrimidin-4-yl]-3-methyl-pyridin- 2-yl}-N,N′-dimethyl-propane-1,3- diamine | 9 | 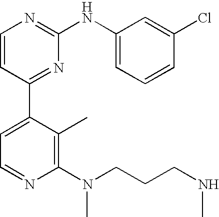 |
| N-[3-({4-[2-(3-Chloro- phenylamino)-pyrimidin-4-yl]-3- methyl-pyridin-2-yl}-methyl- amino)-propyl]-N-methyl- acetamide | 10 | 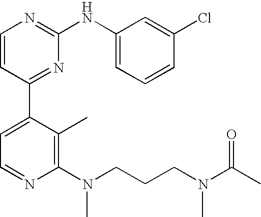 |
| N-(4-{2-[(3-Chloro-phenyl)- methyl-amino]-pyrimidin-4-yl}-3- methyl-pyridin-2-yl)-N,N′- dimethyl-propane-1,3-diamine | 11 | 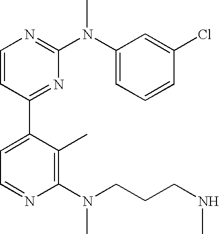 |
| N-{3-[(4-{2-[(3-Chloro-phenyl)- methyl-amino]-pyrimidin-4-yl}-3- methyl-pyridin-2-yl)-methyl- amino]-propyl}-N-methyl- acetamide | 12 | 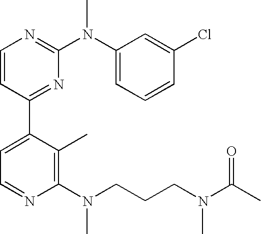 |
| N-[3-(4-{2-[(3-Chloro-phenyl)- methyl-amino]-pyrimidin-4-yl}-3- methyl-pyridin-2-ylamino)- propyl]-acetamide | 13 | 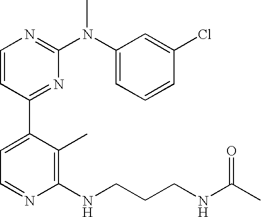 |
| N-(3-{4-[2-(3-Aminomethyl- phenylamino)- pyrimidin-4-yl]-3-methyl- pyridin- 2-ylamino}-propyl)-acetamide | 14 | 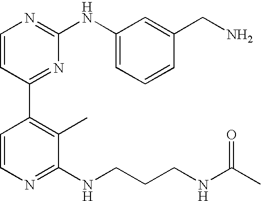 |
| N-[3-(4-{2-[3-(Acetylamino- methyl)-phenylamino]-pyrimidin- 4-yl}-3-methyl-pyridin-2- ylamino)-propyl]-acetamide | 15 | 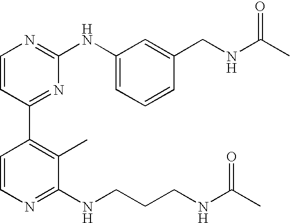 |
| N-(4-{4-[2-(3-Acetylamino- propylamino)-3-methyl-pyridin-4- yl]-pyrimidin-2-ylamino}-benzyl)- acetamide | 16 | 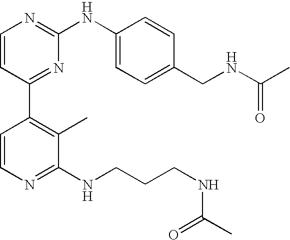 |
| 3-{4-[2-(3-Chloro-phenylamino)- pyrimidin-4-yl]-3-methyl-pyridin- 2-ylamino}-propan-1-ol | 17 | 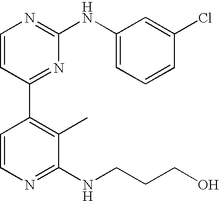 |
| (3-Chloro-phenyl)-{4-[2-(3- methoxy-propylamino)-3-methyl- pyridin-4-yl]-pyrimidin-2-yl}- amine | 18 | 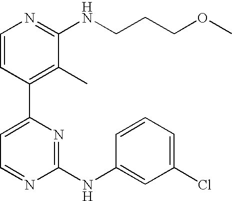 |
| Acetic acid 3-{4-[2-(3-chloro- phenylamino)-pyrimidin-4-yl]-3- methyl-pyridin-2-ylamino}-propyl ester | 19 | 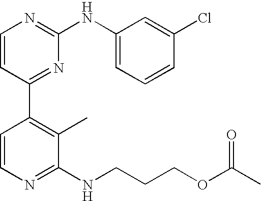 |
| 3-{4-[2-(3-Chloro-phenylamino)- pyrimidin-4-yl]-3-methyl-pyridin- 2-ylamino}-propionic acid | 20 | 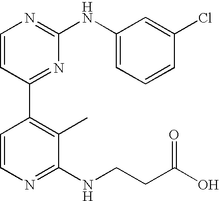 |
| [4-(2-Benzylamino-3-methyl- pyridin-4-yl)-pyrimidin-2-yl]-(3- chloro-phenyl)-amine | 21 | 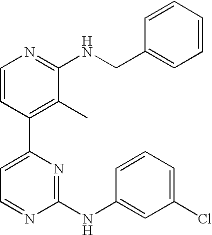 |
| (3-Chloro-phenyl)-{4-[3-methyl- 2-(3-morpholin-4-yl- propylamino)-pyridin-4-yl]- pyrimidin-2-yl}-amine | 22 | 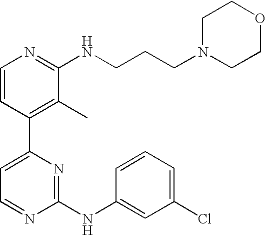 |
| (3-Chloro-phenyl)-{4-[2-(3- imidazol-1-yl-propylamino)-3- methyl-pyridin-4-yl]-pyrimidin-2- yl}-amine | 23 | 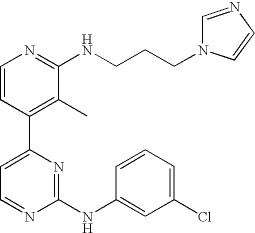 |
| 1-(3-{4-[2-(3-Chloro- phenylamino)-pyrimidin-4-yl]-3- methyl-pyridin-2-ylamino}- propyl)-pyrrolidin-2-one | 24 | 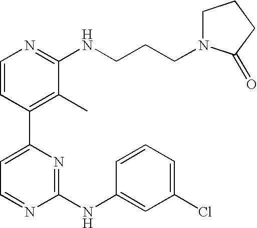 |
| N-(3-{3-Methyl-4-[2-(3-nitro- phenylamino)-pyrimidin-4-yl]- pyridin-2-ylamino}-propyl)- acetamide | 25 | 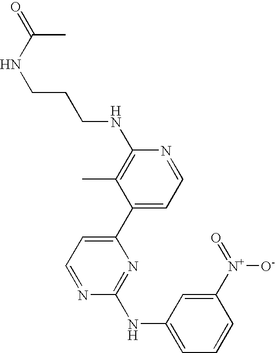 |
| N-(3-{4-[2-(3-Chloro- phenylamino)- pyrimidin- 4-yl]-3-methyl-pyridin-2- ylamino}-propyl)-3-{2-[2-(2- methoxy- ethoxy)-ethoxy]-ethoxy}- propionamide | 26 | 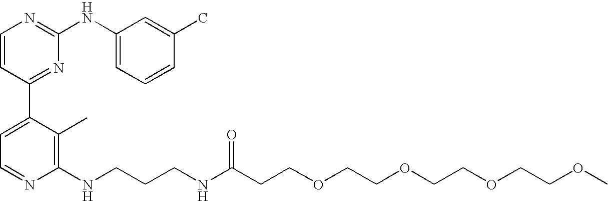 |
| N-(4-Aminomethyl-phenyl)-N′-{4- [2-(3-chloro-phenylamino)- pyrimidin-4-yl]-3-methyl-pyridin- 2-yl}-propane-1,3-diamine | 27 | 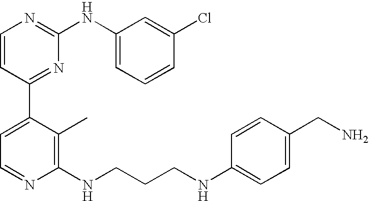 |
| N-[4-(3-{4-[2-(3-Chloro- phenylamino)-pyrimidin-4-yl]-3- methyl-pyridin-2-ylamino}- propylamino)-benzyl]-acetamide | 28 | 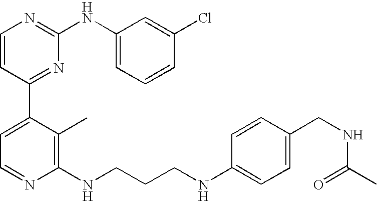 |
| [3-(3-{4-[2-(3-Chloro- phenylamino)-pyrimidin-4-yl]-3- methyl-pyridin-2-ylamino}- propylamino)-phenyl]-phenyl- methanone | 29 | 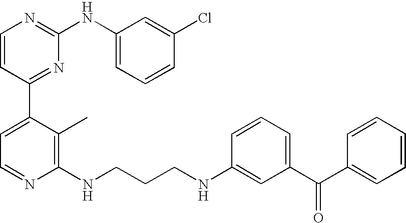 |
| [4-(3-{4-[2-(3-Chloro- phenylamino)-pyrimidin-4-yl]-3- methyl-pyridin-2-ylamino}- propylamino)-phenyl]-phenyl- methanone | 30 | 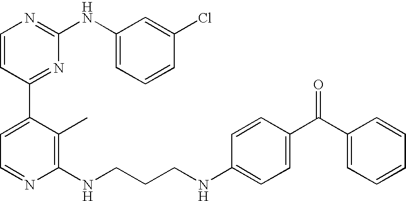 |
| N-{4-[2-(3-Chloro-phenylamino)- 5-methyl-pyrimidin-4-yl]-pyridin- 2-yl}-propane-1,3-diamine | 31 | 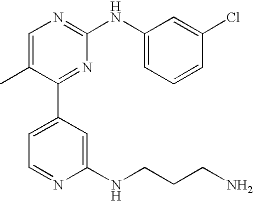 |
| N-(3-{4-[2-(3-Chloro- phenylamino)-5-methyl- pyrimidin-4-yl]-pyridin-2- ylamino}-propyl)-acetamide | 32 | 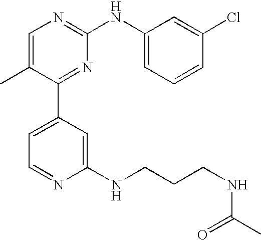 |
| N-{4-[2-(3-Chloro-phenylamino)- 6-methyl-pyrimidin-4-yl]-pyridin- 2-yl}-propane-1,3-diamine | 33 | 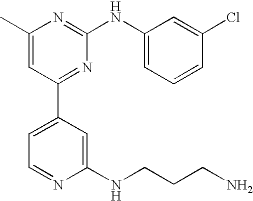 |
| N-(3-{4-[2-(3-Chloro- phenylamino)-6-methyl- pyrimidin-4-yl]-pyridin-2- ylamino}-propyl)-acetamide | 34 | 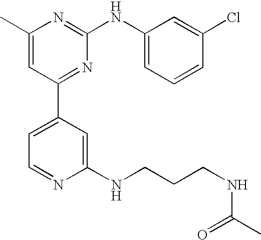 |
| N-(3-Amino-propyl)-N-(3-chloro- phenyl)-9,10-dihydro-2,4,7-triaza- phenanthrene-3,6-diamine | 35 | 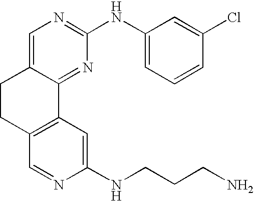 |
| N-{3-[3-(3-Chloro-phenylamino)- 9,10-dihydro-2,4,7-triaza- phenanthren-6-ylamino]-propyl}- acetamide | 36 | 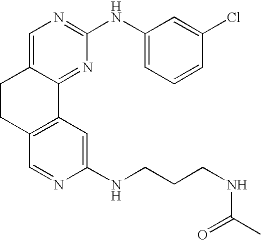 |
| N-(3-Amino-propyl)-N-(3-chloro- phenyl)-9,10-dihydro-2,4,7-triaza- phenanthrene-3,8-diamine | 37 | 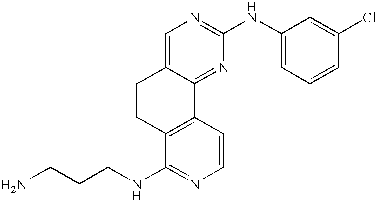 |
| N-{3-[3-(3-Chloro-phenylamino)- 9,10-dihydro-2,4,7-triaza- phenanthren-8-ylamino]-propyl}- acetamide | 38 | 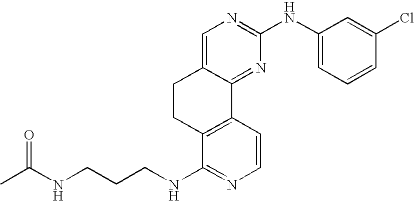 |
| N-{4-[2-(3-Chloro-phenylamino)- pyrimidin-4-yl]-5-methyl-pyridin- 2-yl}-propane-1,3-diamine | 39 | 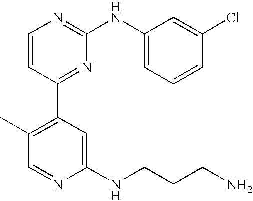 |
| N-(3-{4-[2-(3-Chloro- phenylamino)-pyrimidin-4-yl]-5- methyl-pyridin-2-ylamino}- propyl)-acetamide | 40 | 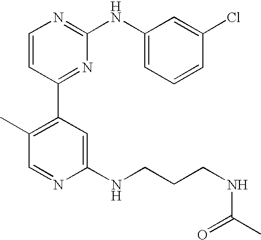 |
| N-{4-[2-(3-Chloro-phenylamino)- pyrimidin-4-yl]-6-methyl-pyridin- 2-yl}-propane-1,3-diamine | 41 | 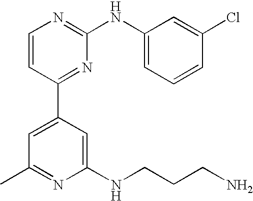 |
| N-(3-{4-[2-(3-Chloro- phenylamino)-pyrimidin-4-yl]-6- methyl-pyridin-2-ylamino}- propyl)-acetamide | 42 | 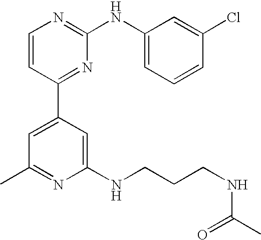 |

TABLE B

| | Compound | |
|---|---|---|
| Compound name | number | Structure |
| (3-Chloro-phenyl)-[4-(2-chloro-pyridin-3-yl)- pyrimidin-2-yl]-amine | 43 | 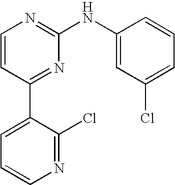 |
| (3-Chloro-phenyl)-[4-(3-methyl-pyridin-4-yl)- pyrimidin-2-yl]-amine | 44 | 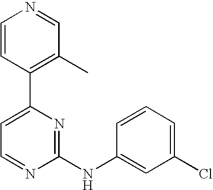 |
| (3-Chloro-phenyl)-[4-(3-methoxy-pyridin-4-yl)- pyrimidin-2-yl]-amine | 45 | 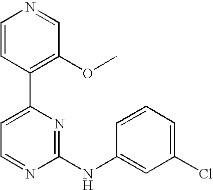 |
| (3-Methanesulfonyl-phenyl)-[4-(3-methoxy- pyridin-4-yl)-pyrimidin-2-yl]-amine | 46 | 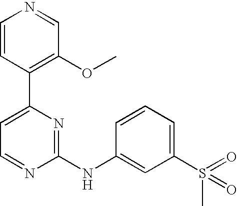 |
| 3-[4-(3-Methoxy-pyridin-4-yl)-5-methyl- pyrimidin-2-ylamino]-benzenesulfonamide | 47 | 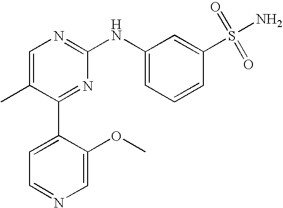 |
| (3-Chloro-phenyl)-(4-quinolin-4-yl-pyrimidin- 2-yl)-amine | 48 | 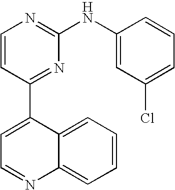 |
| (3-Chloro-phenyl)-[4-(2-dimethylamino- pyridin-3-yl)-pyrimidin-2-yl]-amine | 49 | 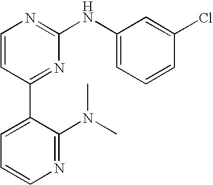 |
| (3-Chloro-phenyl)-{4-[2-(cyclohexylmethyl- amino)-pyridin-3-yl]-pyrimidin-2-yl}-amine | 50 | 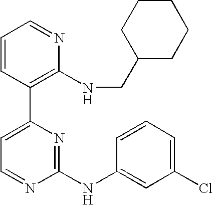 |
| (3-Methanesulfonyl-phenyl)-{4-[2-(3- methanesulfonyl-phenylamino)-pyridin-3-yl]- pyrimidin-2-yl}-amine | 51 | 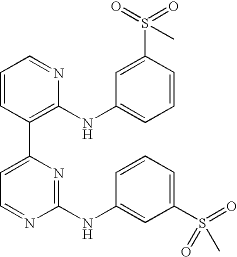 |
| N-{3-[2-(3-Chloro-phenylamino)-pyrimidin-4- yl]-pyridin-2-yl}-ethane-1,2-diamine | 52 | 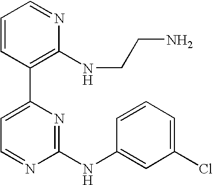 |
| N-{3-[2-(3-Chloro-phenylamino)-pyrimidin-4- yl]-pyridin-2-yl}-N′,N′-dimethyl-ethane-1,2- diamine | 53 | 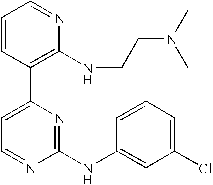 |
| N-(2-{3-[2-(3-Chloro-phenylamino)-pyrimidin- 4-yl]-pyridin-2-ylamino}-ethyl)-acetamide | 54 | 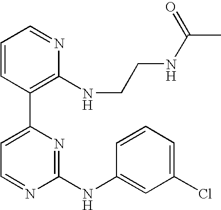 |
| N-{3-[2-(3-Chloro-phenylamino)-pyrimidin-4- yl]-pyridin-2-yl}-propane-1,3-diamine | 55 | 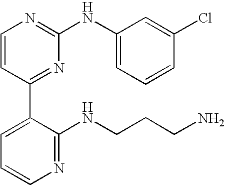 |
| N-{3-[2-(3-Chloro-phenylamino)-5-methyl- pyrimidin-4-yl]-pyridin-2-yl}-propane-1,3- diamine | 56 | 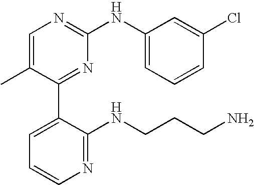 |
| [4-(2-Butylamino-pyridin-3-yl)-5-methyl- pyrimidin-2-yl]-(3-chloro-phenyl)-amine | 57 | 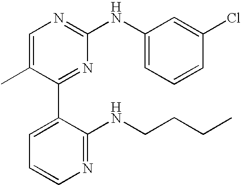 |
| N-{3-[2-(3-Methanesulfonyl-phenylamino)- pyrimidin-4-yl]-pyridin-2-yl}-propane-1,3- diamine | 58 | 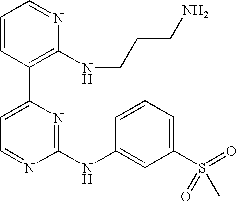 |
| 3-{4-[2-(3-Amino-propylamino)-pyridin-3-yl]- pyrimidin-2-ylamino}-benzenesulfonamide | 59 | 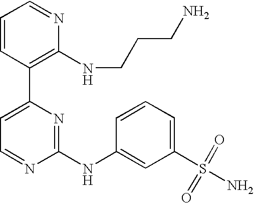 |
| 3-{4-[2-(3-Amino-propylamino)-pyridin-3-yl]- pyrimidin-2-ylamino}-benzonitrile | 60 | 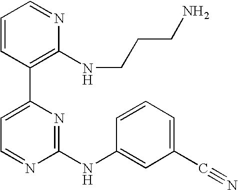 |
| N-{3-[2-(3-Chloro-phenylamino)-pyrimidin-4- yl]-pyridin-2-yl}-2,2-dimethyl-propane-1,3- diamine | 61 | 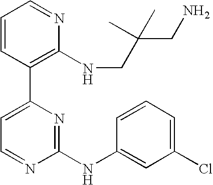 |
| N-{3-[2-(3-Chloro-phenylamino)-pyrimidin-4- yl]-pyridin-2-yl}-N′-methyl-propane-1,3- diamine | 62 | 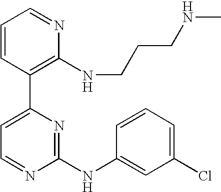 |
| N-{3-[2-(3-Chloro-phenylamino)-pyrimidin-4- yl]-pyridin-2-yl}-N′,N′-dimethyl-propane-1,3- diamine | 63 | 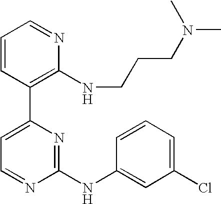 |
| N- {3-[2-(3-Chloro-phenylamino)-pyrimidin-4- yl]-pyridin-2-yl}-N′-isopropyl-propane-1,3- diamine | 64 | 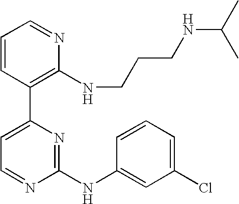 |
| N-(3-{3-[2-(3-Chloro-phenylamino)-pyrimidin- 4-yl]-pyridin-2-ylamino}-propyl)-acetamide | 65 | 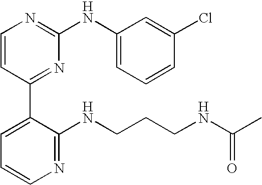 |
| N-(3-{3-[2-(3-Chloro-phenylamino)-5-methyl- pyrimidin-4-yl]-pyridin-2-ylamino}-propyl)- acetamide | 66 | 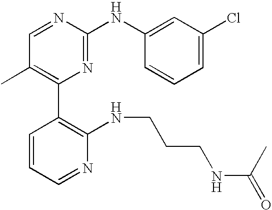 |
| N-(3-{3-[2-(3-Methanesulfonyl-phenylamino)- pyrimidin-4-yl]-pyridin-2-ylamino}-propyl)- acetamide | 67 | 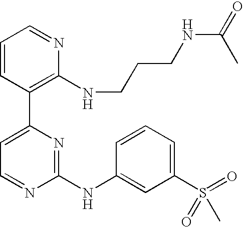 |
| N-(3-{3-[2-(3-Sulfamoyl-phenylamino)- pyrimidin-4-yl]-pyridin-2-ylamino}-propyl)- acetamide | 68 | 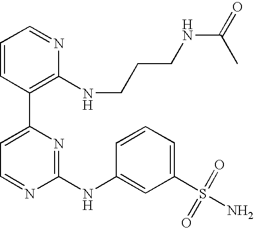 |
| N-(3-{3-[2-(3-Cyano-phenylamino)-pyrimidin- 4-yl]-pyridin-2-ylamino}-propyl)-acetamide | 69 | 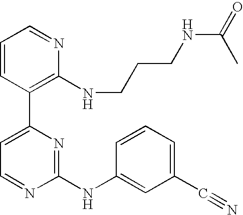 |
| N-(3-{3-[2-(3-Chloro-phenylamino)-pyrimidin- 4-yl]-pyridin-2-ylamino}-propyl)-2,2-dimethyl- propionamide | 70 | 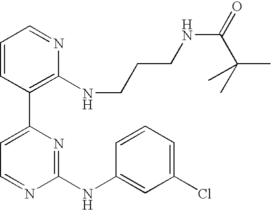 |
| N-(3-{3-[2-(3-Chloro-phenylamino)-pyrimidin- 4-yl]-pyridin-2-ylamino}-propyl)-3,3-dimethyl- butyramide | 71 | 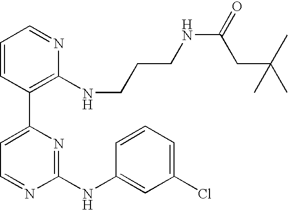 |
| N-(3-{3-[2-(3-Chloro-phenylamino)-pyrimidin- 4-yl]-pyridin-2-ylamino}-propyl)- methanesulfonamide | 72 | 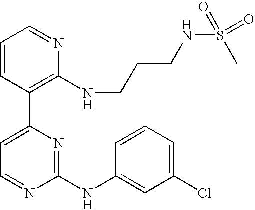 |
| N-(3-{3-[2-(3-Chloro-phenylamino)-pyrimidin- 4-yl]-pyridin-2-ylamino}-2,2-dimethyl-propyl)- acetamide | 73 | 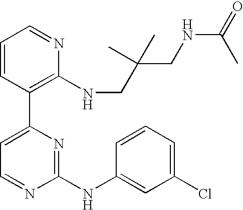 |
| 3-{3-[2-(3-Chloro-phenylamino)-pyrimidin-4- yl]-pyridin-2-ylamino}-propan-1-ol | 74 | 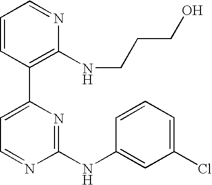 |
| (3-Chloro-phenyl)-{4-[2-(3-methoxy- propylamino)-pyridin-3-yl]-pyrimidin-2-yl}- amine | 75 | 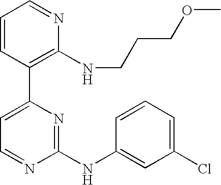 |
| Acetic acid 3-{3-[2-(3-chloro-phenylamino)- pyrimidin-4-yl]-pyridin-2-ylamino}-propyl ester | 76 | 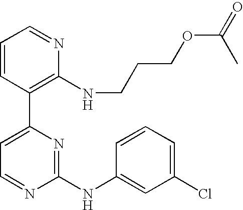 |
| 1-(3-{3-[2-(3-Chloro-phenylamino)-pyrimidin- 4-yl]-pyridin-2-ylamino}-propyl)-pyrrolidin-2- one | 77 | 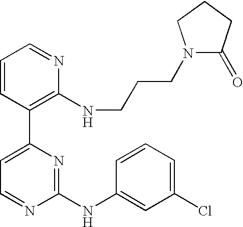 |
| (3-Chloro-phenyl)-{4-[2-(3-morpholin-4-yl- propylamino)-pyridin-3-yl]-pyrimidin-2-yl}- amine | 78 | 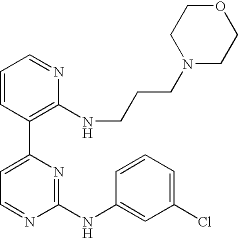 |
| (3-Chloro-phenyl)-{4-[2-(3-imidazol-1-yl- propylamino)-pyridin-3-yl]-pyrimidin-2-yl}- amine | 79 | 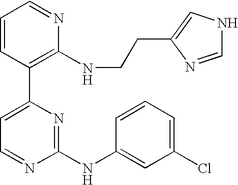 |
| (3-Chloro-phenyl)-{4-[2-(3-imidazol-1-yl- propylamino)-pyridin-3-yl]-pyrimidin-2-yl}- amine | 80 | 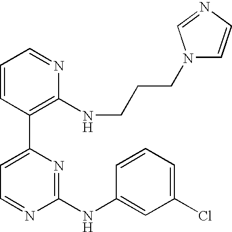 |
| (3-Chloro-phenyl)-(4-{2-[3-(2-methyl-imidazol- 1-yl)-propylamino]-pyridin-3-yl}-pyrimidin-2- yl)-amine | 81 | 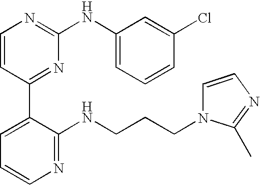 |
| (3-Chloro-phenyl)-(5-methyl-4-{2-[3-(2- methyl-imidazol-1-yl)-propylamino]-pyridin-3- yl}-pyrimidin-2-yl)-amine | 82 | 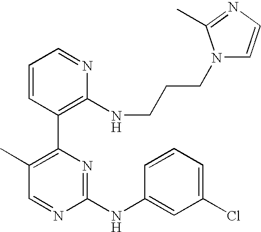 |
| 3-{4-[2-(3-Imidazol-1-yl-propylamino)-pyridin- 3-yl]-pyrimidin-2-ylamino}- benzenesulfonamide | 83 | 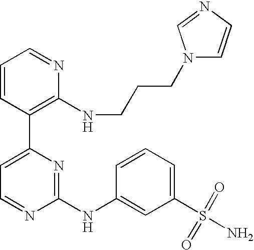 |
| N-{3-[2-(3-Chloro-phenylamino)-pyrimidin-4- yl]-pyridin-2-yl}-butane-1,4-diamine | 84 | 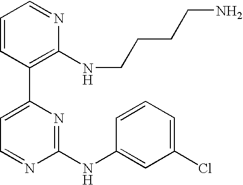 |
| N-(4-{3-[2-(3-Chloro-phenylamino)-pyrimidin- 4-yl]-pyridin-2-ylamino}-butyl)-acetamide | 85 | 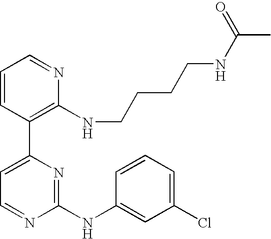 |
| 4-(2-Methoxy-pyridin-3-yl)-pyrimidin-2- ylamine | 86 | 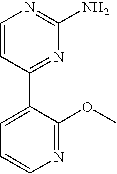 |
| [4-(2-Methoxy-pyridin-3-yl)-pyrimidin-2-yl]- methyl-amine | 87 | 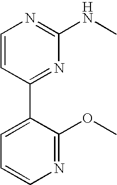 |
| Cyclohexyl-[4-(2-methoxy-pyridin-3-yl)- pyrimidin-2-yl]-amine | 88 | 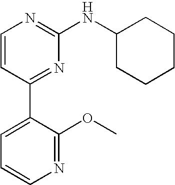 |
| 4-(2-Methoxy-pyridin-3-yl)-pyrimidin-2- ylamine | 89 | 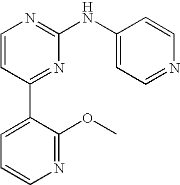 |
| [4-(2-Methoxy-pyridin-3-yl)-pyrimidin-2-yl]- pyridin-3-yl-amine | 90 | 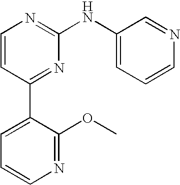 |
| 3-[4-(2-Methoxy-pyridin-3-yl)-pyrimidin-2- ylamino]-benzonitrile | 91 | 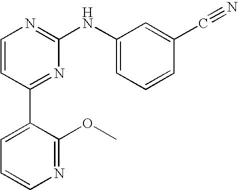 |
| [4-(2-Methoxy-pyridin-3-yl)-pyrimidin-2-yl]-(3- nitro-phenyl)-amine | 92 | 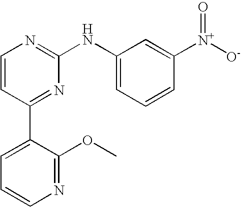 |
| (3-Methanesulfonyl-phenyl)-[4-(2-methoxy- pyridin-3-yl)-pyrimidin-2-yl]-amine | 93 | 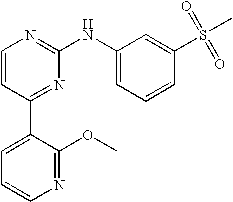 |
| 3-[4-(2-Methoxy-pyridin-3-yl)-pyrimidin-2- ylamino]-benzoic acid | 94 | 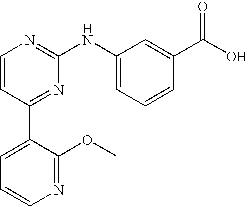 |
| 3-[4-(2-Methoxy-pyridin-3-yl)-pyrimidin-2- ylamino]-benzamide | 95 | 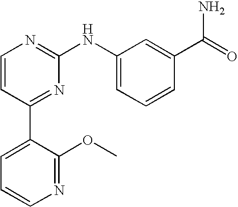 |
| N-Ethyl-3-[4-(2-methoxy-pyridin-3-yl)- pyrimidin-2-ylamino]-benzamide | 96 | 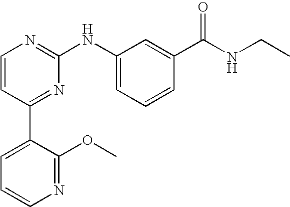 |
| N-(2-Dimethylamino-ethyl)-3-[4-(2-methoxy- pyridin-3-yl)-pyrimidin-2-ylamino]-benzamide | 97 | 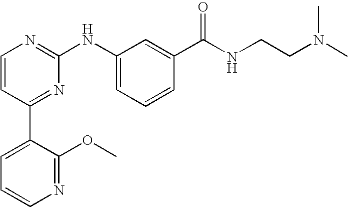 |
| N-{3-[4-(2-Methoxy-pyridin-3-yl)-pyrimidin-2- ylamino]-phenyl}-acetamide | 98 | 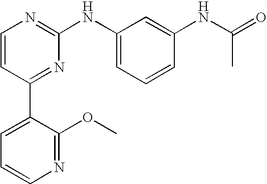 |
| 3-[4-(2-Methoxy-pyridin-3-yl)-pyrimidin-2- ylamino]-benzenesulfonamide | 99 | 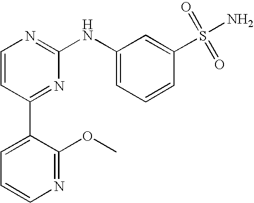 |
| 3-[4-(2-Methoxy-pyridin-3-yl)-pyrimidin-2- ylamino]-N-methyl-benzenesulfonamide | 100 | 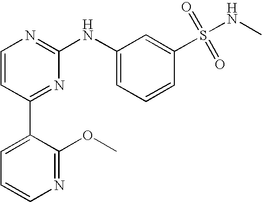 |
| N-Ethyl-3-[4-(2-methoxy-pyridin-3-yl)- pyrimidin-2-ylamino]-benzenesulfonamide | 101 | 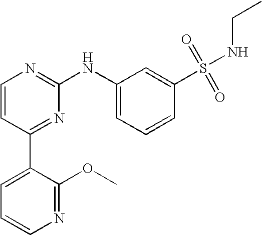 |
| N-Butyl-3-[4-(2-methoxy-pyridin-3-yl)- pyrimidin-2-ylamino]-benzenesulfonamide | 102 | 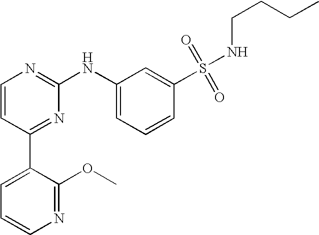 |
| N-(2-Hydroxy-ethyl)-3-[4-(2-methoxy-pyridin- 3-yl)-pyrimidin-2-ylamino]- benzenesulfonamide | 103 | 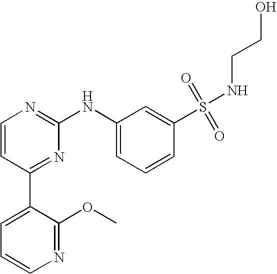 |
| N-Cyclohexyl-3-[4-(2-methoxy-pyridin-3-yl)- pyrimidin-2-ylamino]-benzenesulfonamide | 104 | 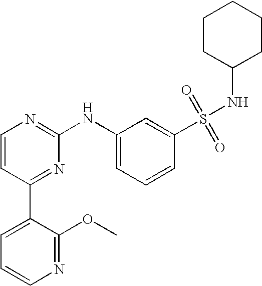 |
| N,N-Diethyl-3-[4-(2-methoxy-pyridin-3-yl)- pyrimidin-2-ylamino]-benzenesulfonamide | 105 | 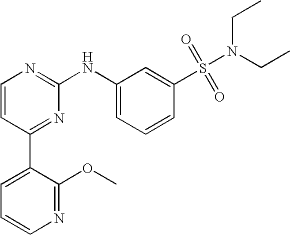 |
| [4-(2-Methoxy-pyridin-3-yl)-pyrimidin-2-yl]-(3- pyrrolidin-1-yl-phenyl)-amine | 106 | 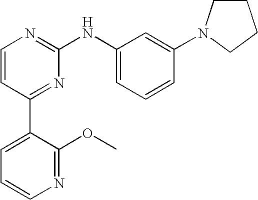 |
| [4-(2-Methoxy-pyridin-3-yl)-pyrimidin-2-yl]-[3- (piperidine-1-sulfonyl)-phenyl]-amine | 107 | 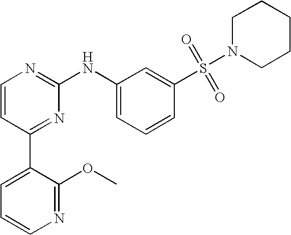 |
| 3-[4-(2-Methoxy-pyridin-3-yl)-pyrimidin-2- ylamino]-benzoic acid ethyl ester | 108 | 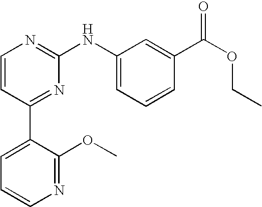 |
| 3-[4-(2-Methoxy-pyridin-3-yl)-pyrimidin-2- ylamino]-benzoic acid tert-butyl ester | 109 | 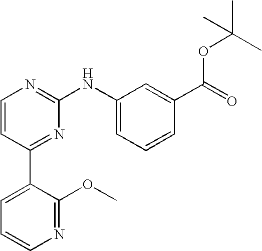 |
| [4-(2-Methoxy-pyridin-3-yl)-pyrimidin-2-yl]-(3- oxazol-5-yl-phenyl)-amine | 110 | 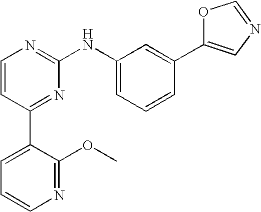 |
| [4-(2-Methoxy-pyridin-3-yl)-pyrimidin-2-yl]-[3- (4-methyl-4H-[1,2,4]triazo1-3-yl)-phenyl]-amine | 111 | 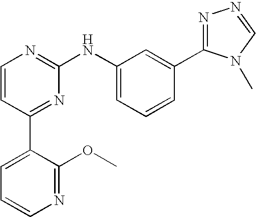 |
| (1H-Indazol-6-yl)-[4-(2-methoxy-pyridin-3-yl)- pyrimidin-2-yl]-amine | 112 | 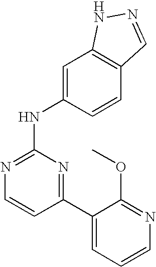 |
| [4-(2-Methoxy-pyridin-3-yl)-pyrimidin-2-yl]-(3- piperidin-4-yl-phenyl)-amine | 113 | 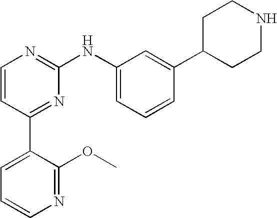 |
| 4-{3-[4-(2-Methoxy-pyridin-3-yl)-pyrimidin-2- ylamino]-phenyl}-piperidine-1-carboxylic acid tert-butyl ester | 114 | 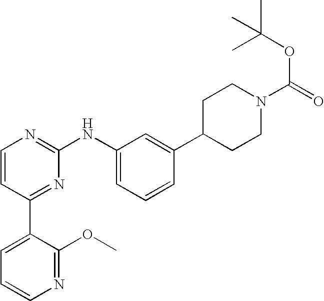 |
| Biphenyl-3-yl-[4-(2-methoxy-pyridin-3-yl)- pyrimidin-2-yl]-amine | 115 | 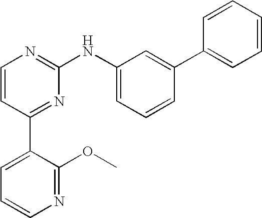 |
| [4-(2-Methoxy-pyridin-3-yl)-pyrimidin-2-yl]-(4- nitro-phenyl)-amine | 116 | 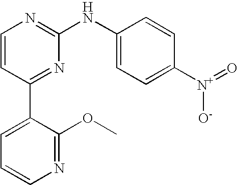 |
| [4-(2-Methoxy-pyridin-3-yl)-pyrimidin-2-yl]-[4- (4-methyl-piperazin-1-yl)-phenyl]-amine | 117 | 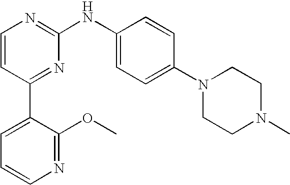 |
| (3-Chloro-phenyl)-[4-(2,6-dimethoxy-pyridin-3- yl)-pyrimidin-2-yl]-amine | 118 | 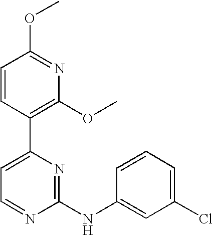 |
| [4-(2,6-Dimethoxy-pyridin-3-yl)-pyrimidin-2- yl]-(3-methanesulfonyl-phenyl)-amine | 119 | 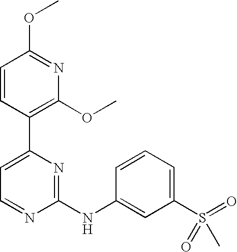 |
| {4-[2-(3-Amino-propoxy)-pyridin-3-yl]- pyrimidin-2-yl}-(3-chloro-phenyl)-amine | 120 | 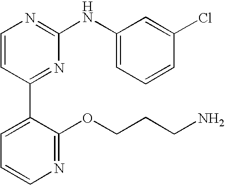 |
| N-(3-{3-[2-(3-Chloro-phenylamino)-pyrimidin- 4-yl]-pyridin-2-yloxy}-propyl)-acetamide | 121 | 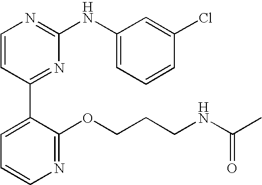 |

TABLE C

| | Compound | |
|---|---|---|
| Compound name | number | Structure |
| N-{3-[2-(3-Chloro- phenylamino)-pyrimidin-4- yl]-pyridin-2-yl}-acetamide | 123 | 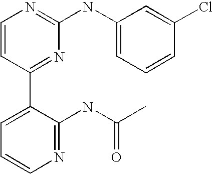 |
| (3-Chloro-phenyl)-[4-(2- methyl-pyridin-3-yl)- pyrimidin-2-yl]-amine | 124 | 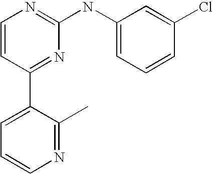 |
| (Z)-2-[2-(3-Chloro- phenylamino)-pyrimidin-4- yl]-N-methyl-penta-2,4- dienimidoyl fluoride | 125 | 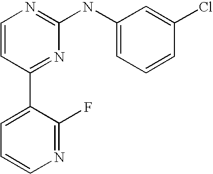 |
| [4-(2-Bromo-pyridin-3-yl)- pyrimidin-2-yl]-(3-chloro- phenyl)-amine | 126 | 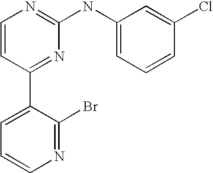 |
| 3-[2-(3-Chloro- phenylamino)-pyrimidin-4- yl]-pyridine-2-carbonitrile | 127 | 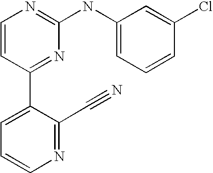 |
| (3-Chloro-phenyl)-[4-(2- ethoxy-pyridin-3-yl)- pyrimidin-2-yl]-amine | 128 | 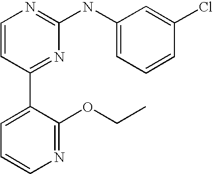 |
| (3-Chloro-phenyl)-[4-(2- ethyl-pyridin-3-yl)- pyrimidin-2-yl]-amine | 129 | 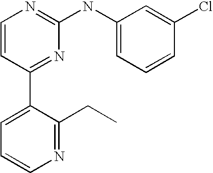 |
| (3-Chloro-phenyl)-[5- methyl-4-(2-methyl- pyridin-3-yl)-pyrimidin-2- yl]-amine | 130 | 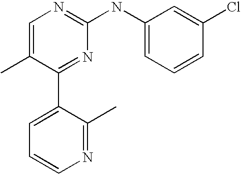 |
| (3-Chloro-phenyl)-[4-(2,5- dimethyl-pyridin-3-yl)- pyrimidin-2-yl]-amine | 131 | 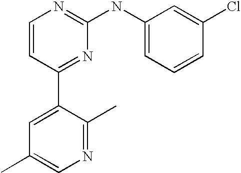 |
| (3-Chloro-phenyl)-[4-(6- fluoro-2-methoxy-pyridin- 3-yl)-pyrimidin-2-yl]amine | 132 | 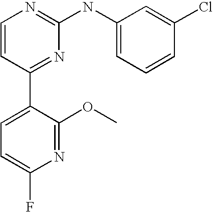 |
| N-{3-[2′-(3-Chloro- phenylamino)- [3,4]bipyridinyl-2- ylamino]-propyl}- acetamide | 133 | 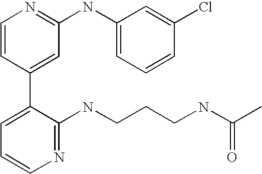 |
| N-(3-{3-[6-(3-Chloro- phenylamino)-pyrimidin-4- yl]-pyridin-2-ylamino}- propyl)-acetamide | 134 | 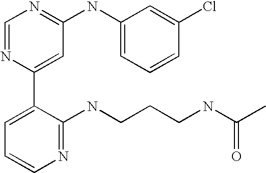 |
| N-(3-{3-[4-(3-Chloro- phenylamino)- [1,3,5]triazin-2-yl]-pyridin- 2-ylamino}-propyl)- acetamide | 135 | 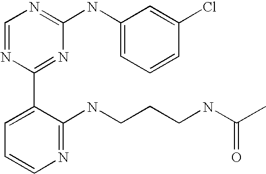 |
| (3-Chloro-phenyl)-[4-(2,5- dimethyl-pyridin-4-yl)- pyrimidin-2-yl]-amine | 136 | 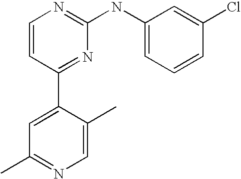 |
| (3-Chloro-phenyl)-[5- methyl-4-(3-methyl- pyridin-4-yl)-pyrimidin-2- yl]-amine | 137 | 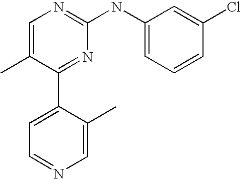 |
| (3-Chloro-phenyl)-[4-(2,3- dimethyl-pyridin-4-yl)- pyrimidin-2-yl]-amine | 138 | 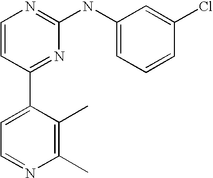 |
| (3-Chloro-phenyl)-[5- fluoro-4-(3-methyl-pyridin- 4-yl)-pyrimidin-2-yl]-amine | 139 | 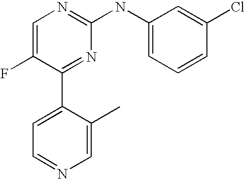 |
| [5-Bromo-4-(3-methyl- pyridin-4-yl)-pyrimidin-2- yl]-(3-chloro-phenyl)-amine | 140 | 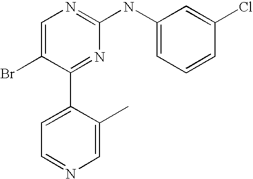 |
| (3-Chloro-phenyl)-[5- methoxy-4-(3-methyl- pyridin-4-yl)-pyrimidin-2- yl]amine | 141 | 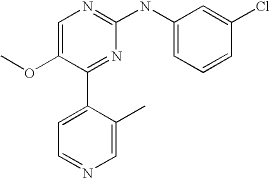 |
| N-(3-Chloro-phenyl)-4-(3- methyl-pyridin-4-yl)- pyrimidine-2,5-diamine | 142 | 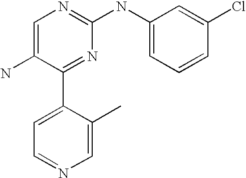 |
| N-{4-[2-(3-Chloro- phenylamino)-pyrimidin-4- yl]-pyridin-3-yl}-propane- 1,3-diamine | 143 | 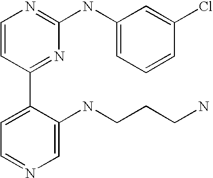 |
| N-(3-{4-[2-(3-Chloro- phenylamino)-pyrimidin-4- yl]-pyridin-3-ylamino}- propyl)-acetamide | 144 | 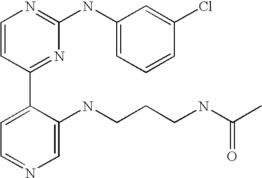 |
| (3-Chloro-phenyl)-{4-[3-(3- imidazol-1-yl- propylamino)-pyridin-4-yl]- pyrimidin-2-yl}-amine | 145 | 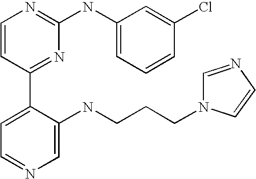 |
| N-(3-{4-[2-(3-Chloro- phenylamino)-pyrimidin-4- yl]-2-methoxy-pyridin-3- ylamino}-propyl)- acetamide | 146 | 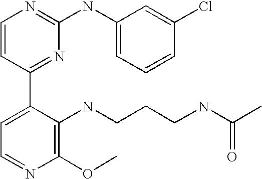 |
| N-(3-{4-[2-(3-Chloro- phenylamino)-pyrimidin-4- yl]-2-methyl-pyridin-3- ylamino}-propyl)- acetamide | 147 | 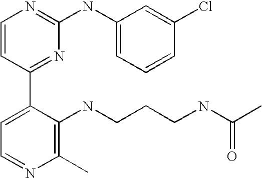 |
| N-(3-{4-[2-(3-Chloro- phenylamino)-pyrimidin-4- yl]-2-methylamino-pyridin- 3-ylamino}-propyl)- acetamide | 148 | 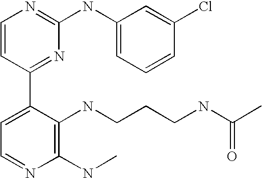 |
| N-(3-{4-[2-(3-Imidazol-1- yl-phenylamino)-pyrimidin- 4-yl]-pyridin-3-ylamino}- propyl)-acetamide | 149 | 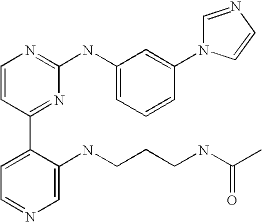 |
| N-(3-{2-Chloro-4-[2-(3- chloro-phenylamino)- pyrimidin-4-yl]-pyridin-3- ylamino}-propyl)- acetamide | 150 | 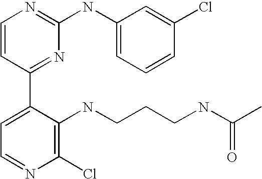 |
| N-{3-[2′-(3-Chloro- phenylamino)- [4,4′]bipyridinyl-3- ylamino]-propyl}- acetamide | 151 | 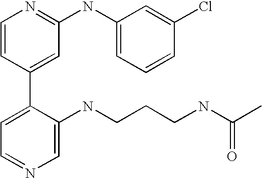 |
| N-(3-{4-[6-(3-Chloro- phenylamino)-pyrimidin-4- yl]-pyridin-3-ylamino}- propyl)-acetamide | 152 | 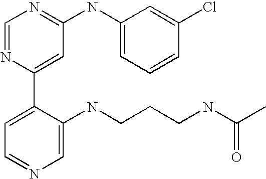 |
| N-(3-{4-[4-(3-Chloro- phenylamino)- [1,3,5]triazin-2-yl]-pyridin- 3-ylamino}-propyl)- acetamide | 153 | 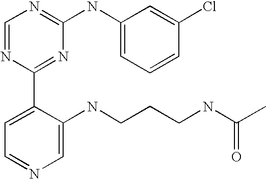 |
| N-(3-{4-[2-(3-Chloro- phenylamino)-pyrimidin-4- yl]-pyridazin-3- ylamino}propyl)-acetamide | 154 | 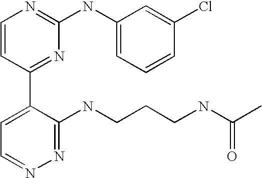 |
| (3-Chloro-phenyl)-[4-(3- chloro-pyridazin-4-yl)- pyrimidin-2-yl]-amine | 155 | 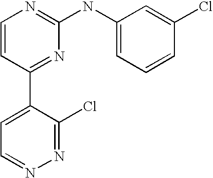 |
| (3-Chloro-phenyl)-[4-(3- methyl-pyridazin-4-yl)- pyrimidin-2-yl]-amine | 156 | 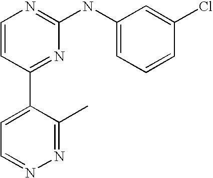 |
| (3-Chloro-phenyl)-[4-(3- methoxy-pyridazin-4-yl)- pyrimidin-2-yl]-amine | 157 | 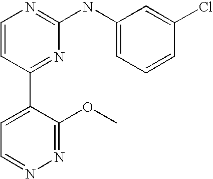 |
| N-(3-{4-[2-(3-Chloro- phenylamino)-pyridin-4- yl]-pyridazin-3-ylamino}- propyl)-acetamide | 158 | 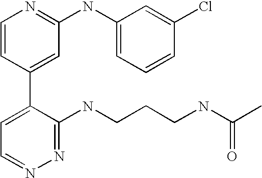 |
| N-(3-{4-[6-(3-Chloro- phenylamino)-pyrimidin-4- yl]-pyridazin-3-ylamino}- propyl)-acetamide | 159 | 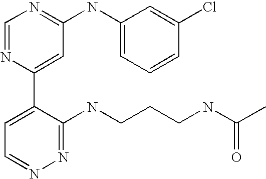 |
| N-(3-{4-[4-(3-Chloro- phenylamino)- [1,3,5]triazin-2-yl]- pyridazin-3-ylamino}- propyl)-acetamide | 160 | 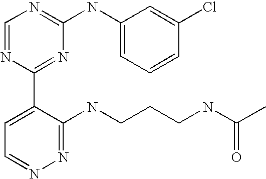 |

In certain embodiments, the compound of the present invention is further characterized as a modulator of a protein kinase, including, but not limited to, protein kinases selected from the group consisting of abl, ATK, Bcr-abl, Blk, Brk, Btk, c-fms, e-kit, c-met, c-src, CDK, cRaf1, CSFIR, CSK, EGFR, ErbB2, ErbB3, ErbB4, ERK, Fak, fes, FGFRI, FGFR2, FGFR3, FGFR4, FGFR5, Fgr, FLK-4, flt-1, Fps, Frk, Fyn, GSK, Gst-Flk1, Hck, Her-2, Her-4, IGF-1R, INS-R, Jak, JNK, KDR, Lck, Lyn, MEK, p38, panHER, PDGFR, PLK, PKC, PYK2, Raf, Rho, ros, SRC, TRK, TYK2, UL97, VEGFR, Yes, Zap70, Aurora-A, GSK3-alpha, HIPK1, HIPK2, HIP3, IRAK1, JNK1, JNK2, JNK3, TRKB, CAMKII, CK1, CK2, RAF, GSK3Beta, MAPK1, MKK4, MKK7, MST2, NEK2, AAK1, PKCalpha, PKD, RIPK2 and ROCK-II.

In a preferred embodiment, the protein kinase is selected from the group consisting of CDK1, CDK2, CDK3, CDK4, CDK5, CDK6, CDK7, CDK8 and CDK9 and any combination thereof, as well as any other CDK, as well as any CDK not yet identified. In a particularly preferred embodiment, the protein kinase is selected from the group consisting of CDK1, CDK2 and CDK9.

In a particular embodiment, CDK combinations of interest include CDK4 and CDK9; CDK1, CDK2 and CDK9; CDK9 and CDK7; CDK9 and CDK1; CDK9 and CDK2; CDK4, CDK6 and CDK9; CDK1, CDK2, CDK3, CDK4, CDK6 and CDK9.

In other embodiments, the compounds of the present invention are used for the treatment of protein kinase-associated disorders. As used herein, the term “protein kinase-associated disorder” includes disorders and states (e.g., a disease state) that are associated with the activity of a protein kinase, e.g., the CDKs, e.g., CDK1, CDK2 and/or CDK9. Non-limiting examples of protein kinase-associated disorders include abnormal cell proliferation (including protein kinase-associated cancers), viral infections, fungal infections, autoimmune diseases and neurodegenerative disorders.

Non-limiting examples of protein-kinase associated disorders include proliferative diseases, such as viral infections, auto-immune diseases, fungal disease, cancer, psoriasis, vascular smooth cell proliferation associated with atherosclerosis, pulmonary fibrosis, arthritis glomerulonephritis, chronic inflammation, neurodegenerative disorders, such as Alzheimer's disease, and post-surgical stenosis and restenosis. Protein kinase-associated diseases also include diseases related to abnormal cell proliferation, including, but not limited to, cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenocarcinoma, adenoma, adenocarcinoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's, hairy cells, and leukemia.

Additional non-limiting examples of protein kinase-associated cancers include carcinomas, hematopoietic tumors of lymphoid lineage, hematopoietic tumors of myeloid lineage, tumors of mesenchymal origin, tumors of the central and peripheral nervous system, melanoma, seminoma, teratocarcinoma, osteosarcoma, xenoderoma pigmentosum, keratoctanthoma, thyroid follicular cancer and Kaposi's sarcoma.

Protein kinase-associated disorders include diseases associated with apoptosis, including, but not limited to, cancer, viral infections, autoimmune diseases and neurodegenerative disorders.

Non-limiting examples of protein-kinase associated disorders include viral infections in a patient in need thereof, wherein the viral infections include, but are not limited to, HIV, human papilloma virus, herpes virus, poxvirus, Epstein-Barr virus, Sindbis virus and adenovirus.

Non-limiting examples of protein-kinase associated disorders include tumor angiogenesis and metastasis. Non-limiting examples of protein-kinase associated disorders also include vascular smooth muscle proliferation associated with atherosclerosis, postsurgical vascular stenosis and restenosis, and endometriosis.

Further non-limiting examples of protein-kinase associated disorders include those associated with infectious agents, including yeast, fungi, protozoan parasites such as Plasitiodium falciparum, and DNA and RNA viruses.

In another embodiment, the compound of the present invention is further characterized as a modulator of a combination of protein kinases, e.g., the CDKs, e.g., CDK1, CDK2 and/or CDK9. In certain embodiments, a compound of the present invention is used for protein kinase-associated diseases, and/or as an inhibitor of any one or more protein kinases. It is envisioned that a use can be a treatment of inhibiting one or more isoforms of protein kinases.

The compounds of the invention are inhibitors of cyclin-dependent kinase enzymes. Without being bound by theory, inhibition of the CDK4/cyclin D1 complex blocks phosphorylation of the Rb/inactive E2F complex, thereby preventing release of activated E2F and ultimately blocking E2F-dependent DNA transcription. This has the effect of inducing G₁ cell cycle arrest. In particular, the CDK4 pathway has been shown to have tumor-specific deregulation and cytotoxic effects. Accordingly, the ability to inhibit the activity of combinations of CDKs will be of beneficial therapeutic use.

Furthermore, the cell's ability to respond and survive chemotherapeutic assault may depend on rapid changes in transcription or on activation of pathways which are highly sensitive to CDK9/cyclinT1 (PTEF-b) activity. CDK9 inhibition may sensitize cells to TNFalpha or TRAIL stimulation by inhibition of NF-kB, or may block growth of cells by reducing myc-dependent gene expression. CDK9 inhibition may also sensitize cells to genotoxic chemotherapies, HDAC inhibition, or other signal transduction based therapies.

As such, the compounds of the invention can lead to depletion of anti-apoptotic proteins, which can directly induce apoptosis or sensitize to other apoptotic stimuli, such as cell cycle inhibition, DNA or microtubule damage or signal transduction inhibition. Depletion of anti-apoptotic proteins by the compounds of the invention may directly induce apoptosis or sensitize to other apoptotic stimuli, such as cell cycle inhibition, DNA or microtubule damage or signal transduction inhibition.

The compounds of the invention can be effective in combination with chemotherapy, DNA damage arresting agents, or other cell cycle arresting agents. The compounds of the invention can also be effective for use in chemotherapy-resistant cells.

The present invention includes treatment of one or more symptoms of cancer, inflammation, cardiac hypertrophy, and HIV infection, as well as protein kinase-associated disorders as described above, but the invention is not intended to be limited to the manner by which the compound performs its intended function of treatment of a disease. The present invention includes treatment of diseases described herein in any manner that allows treatment to occur, e.g., cancer, inflammation, cardiac hypertrophy, and HIV infection.

In certain embodiments, the invention provides a pharmaceutical composition of any of the compounds of the present invention. In a related embodiment, the invention provides a pharmaceutical composition of any of the compounds of the present invention and a pharmaceutically acceptable carrier or excipient of any of these compounds. In certain embodiments, the invention includes the compounds as novel chemical entities.

In one embodiment, the invention includes a packaged protein kinase-associated disorder treatment. The packaged treatment includes a compound of the invention packaged with instructions for using an effective amount of the compound of the invention for an intended use.

The compounds of the present invention are suitable as active agents in pharmaceutical compositions that are efficacious particularly for treating protein kinase-associated disorders, e.g., cancer, inflammation, cardiac hypertrophy, and HIV infection. The pharmaceutical composition in various embodiments has a pharmaceutically effective amount of the present active agent along with other pharmaceutically acceptable excipients, carriers, fillers, diluents and the like. The phrase, “pharmaceutically effective amount” as used herein indicates an amount necessary to administer to a host, or to a cell, issue, or organ of a host, to achieve a therapeutic result, especially the regulating, modulating, or inhibiting protein kinase activity, e.g., inhibition of the activity of a protein kinase, or treatment of cancer, inflammation, cardiac hypertrophy, and HIV infection.

In other embodiments, the present invention provides a method for inhibiting the activity of a protein kinase. The method includes contacting a cell with any of the compounds of the present invention. In a related embodiment, the method further provides that the compound is present in an amount effective to selectively inhibit the activity of a protein kinase.

In other embodiments, the present invention provides a use of any of the compounds of the invention for manufacture of a medicament to treat cancer, inflammation, cardiac hypertrophy, and HIV infection in a subject.

In other embodiments, the invention provides a method of manufacture of a medicament, including formulating any of the compounds of the present invention for treatment of a subject.

### DEFINITIONS

The term “treat,” “treated,” “treating” or “treatment” includes the diminishment or alleviation of at least one symptom associated or caused by the state, disorder or disease being treated. In certain embodiments, the treatment comprises the induction of a protein kinase-associated disorder, followed by the activation of the compound of the invention, which would in turn diminish or alleviate at least one symptom associated or caused by the protein kinase-associated disorder being treated. For example, treatment can be diminishment of one or several symptoms of a disorder or complete eradication of a disorder.

The term “use” includes any one or more of the following embodiments of the invention, respectively: the use in the treatment of protein kinase-associated disorders; the use for the manufacture of pharmaceutical compositions for use in the treatment of these diseases, e.g., in the manufacture of a medicament; methods of use of compounds of the invention in the treatment of these diseases; pharmaceutical preparations having compounds of the invention for the treatment of these diseases; and compounds of the invention for use in the treatment of these diseases; as appropriate and expedient, if not stated otherwise. In particular, diseases to be treated and are thus preferred for use of a compound of the present invention are selected from cancer, inflammation, cardiac hypertrophy, and HIV infection, as well as those diseases that depend on the activity of protein kinases. The term “use” further includes embodiments of compositions herein which bind to a protein kinase sufficiently to serve as tracers or labels, so that when coupled to a fluor or tag, or made radioactive, can be used as a research reagent or as a diagnostic or an imaging agent.

The term “subject” is intended to include organisms, e.g., prokaryotes and eukaryotes, which are capable of suffering from or afflicted with a disease, disorder or condition associated with the activity of a protein kinase. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from cancer, inflammation, cardiac hypertrophy, and HIV infection, and other diseases or conditions described herein (e.g., a protein kinase-associated disorder). In another embodiment, the subject is a cell.

The language “protein kinase-modulating compound,” “modulator of protein kinase” or “protein kinase inhibitor” refers to compounds that modulate, e.g., inhibit, or otherwise alter, the activity of a protein kinase. Examples of protein kinase-modulating compounds include compounds of the invention, i.e., Formula I and Formula II, as well as the compounds of Table A, Table B, and Table C (including pharmaceutically acceptable salts thereof, as well as enantiomers, stereoisomers, rotamers, tautomers, diastereomers, atropisomers or racemates thereof).

Additionally, a method of the invention includes administering to a subject an effective amount of a protein kinase-modulating compound of the invention, e.g., protein kinase-modulating compounds of Formula I and Formula II, as well as Table A, Table B, and Table C (including pharmaceutically acceptable salts thereof, as well as enantiomers, stereoisomers, rotamers, tautomers, diastereomers, atropisomers or racemates thereof).

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term “alkyl” also includes alkenyl groups and alkynyl groups. Furthermore, the expression “Cₓ—C_{y}-alkyl”, wherein x is 1-5 and y is 2-10 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl, and isobutyl and sec-butyl. Moreover, the term C₃₋₇-cycloalkyl includes, but is not limited to, cyclopropyl, cyclopentyl, cyclohexyl and cycloheptyl. As discussed below, these alkyl groups, as well as cycloalkyl groups, may be further substituted.

The term alkyl further includes alkyl groups which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer carbons. Likewise, preferred cycloalkyls have from 4-7 carbon atoms in their ring structure, and more preferably have 5 or 6 carbons in the ring structure.

Moreover, alkyl (e.g., methyl, ethyl, propyl, butyl, pentyl, hexyl, etc.) includes both “unsubstituted alkyl” and “substituted alkyl”, the latter of which refers to alkyl moieties having substituents replacing a hydrogen on one or more carbons of the hydrocarbon backbone, which allow the molecule to perform its intended function.

The term “substituted” is intended to describe moieties having substituents replacing a hydrogen on one or more atoms, e.g. C, O or N, of a molecule. Such substitutents can include electron-withdrawing groups or electron-withdrawing atoms. Such substituents can include, for example, oxo, alkyl, alkoxy, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkyl amino, dialkylamino, arylamino, diarylamino, and alkylarylamino), acylamino (including alkylcarbonylamino, arylcarbonylamino, carbamoyl and ureido), amidino, imino, sulflhydryl, alkylthio, arylthio, thiocarboxylate, sulfates, alkylsulfinyl, sulfonato, sulfamoyl, sulfonamido, nitro, trifluoromethyl, cyano, azido, heterocyclic, alkylaryl, morpholino, phenol, benzyl, phenyl, piperizine, cyclopentane, cyclohexane, pyridine, 5H-tetrazole, triazole, piperidine, or an aromatic or heteroaromatic moiety, and any combination thereof.

Further examples of substituents of the invention, which are not intended to be limiting, include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR, (CR′R″)₀₋₃ (substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, oxime, thiol, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety, and any combination thereof. In certain embodiments, a carbonyl moiety (C═O) can be further derivatized with an oxime moiety, e.g., an aldehyde moiety can be derivatized as its oxime (—C═N—OH) analog. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

The term “alkenyl” includes unsaturated aliphatic groups analogous in length and possible substitution to the alkyls described above, but which contain at least one double bond.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulflhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “amine” or “amino” should be understood as being broadly applied to both a molecule, or a moiety or functional group, as generally understood in the art, and can be primary, secondary, or tertiary. The term “amine” or “amino” includes compounds where a nitrogen atom is covalently bonded to at least one carbon, hydrogen or heteroatom. The terms include, for example, but are not limited to, “alkyl amino,” “arylamino,” “diarylamino,” “alkylarylamino,” “alkylaminoaryl,” “arylaminoalkyl,” “alkaminoalkyl,” “amide,” “amido,” and “aminocarbonyl.” The term “alkyl amino” comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonyl amino).

In a particular embodiment of the invention, the term “amine” or “amino” refers to substituents of the formulas N(R⁸)R⁹ or C₁₋₆—N(R⁸)R⁹, wherein R⁸ and R⁹ are each, independently, selected from the group consisting of —H and —(C₁₋₆alkyl)₀₋₁G, wherein G is selected from the group consisting of H, COOH, NH₂, N(H)C(O)C₁₋₆alkyl, N(C₁₋₆alkyl)C(O)C₁₋₆alkyl, N(H)C₁₋₆alkyl, OH, OC(O)C₁₋₆alkyl, C₃₋₇-cycloalkyl, phenyl, substituted phenyl, C(O)OC₁-C₆-alkyl, C(O)C₁₋₆alkyl-COOH, C(O)C₁-C₄-alkyl, C(O)-aryl, morpholino, imidazole, pyrrolidin-2-one, substituted or unsubstituted heterocycle, pyrazole, pyridine, oxazole, thiazole, isoxazole, triazole, tetrazole, pyrimidine, pyridazine, pyrazine, piperazine and piperidine.

The term “aryl” includes groups, including 5- and 6-membered single-ring aromatic groups that can include from zero to four heteroatoms, for example, phenyl, pyrrole, furan, thiophene, thiazole, isothiaozole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, anthryl, phenanthryl, napthridine, indole, benzofuran, purine, benzofuran, deazapurine, or indolizine. Those aryl groups having heteroatoms in the ring structure can also be referred to as “aryl heterocycles”, “heterocycles,” “heteroaryls” or “heteroaromatics.” The aromatic ring can be substituted at one or more ring positions with such substituents as described above, as for example, alkyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrahydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

The term “acyl” includes compounds and moieties which contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups and may include cyclic groups such as cyclopentoxy. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “carbonyl” or “carboxy” includes compounds and moieties which contain a carbon connected with a double bond to an oxygen atom, and tautomeric forms thereof. Examples of moieties that contain a carbonyl include aldehydes, ketones, carboxylic acids, amides, esters, anhydrides, etc. The term “carboxy moiety” or “carbonyl moiety” refers to groups such as “alkylcarbonyl” groups wherein an alkyl group is covalently bound to a carbonyl group, “alkenylcarbonyl” groups wherein an alkenyl group is covalently bound to a carbonyl group, “alkynylcarbonyl” groups wherein an alkynyl group is covalently bound to a carbonyl group, “arylcarbonyl” groups wherein an aryl group is covalently attached to the carbonyl group. Furthermore, the term also refers to groups wherein one or more heteroatoms are covalently bonded to the carbonyl moiety. For example, the term includes moieties such as, for example, aminocarbonyl moieties, (wherein a nitrogen atom is bound to the carbon of the carbonyl group, e.g., an amide), aminocarbonyloxy moieties, wherein an oxygen and a nitrogen atom are both bond to the carbon of the carbonyl group (e.g., also referred to as a “carbamate”). Furthermore, aminocarbonylamino groups (e.g., ureas) are also include as well as other combinations of carbonyl groups bound to heteroatoms (e.g., nitrogen, oxygen, sulfur, etc. as well as carbon atoms). Furthermore, the heteroatom can be further substituted with one or more alkyl, alkenyl, alkynyl, aryl, aralkyl, acyl, etc. moieties.

The term “thiocarbonyl” or “thiocarboxy” includes compounds and moieties which contain a carbon connected with a double bond to a sulfur atom. The term “thiocarbonyl moiety” includes moieties that are analogous to carbonyl moieties. For example, “thiocarbonyl” moieties include aminothiocarbonyl, wherein an amino group is bound to the carbon atom of the thiocarbonyl group, furthermore other thiocarbonyl moieties include, oxythiocarbonyls (oxygen bound to the carbon atom), aminothiocarbonylamino groups, etc.

The term “ether” includes compounds or moieties that contain an oxygen bonded to two different carbon atoms or heteroatoms. For example, the term includes “alkoxyalkyl” which refers to an alkyl, alkenyl, or alkynyl group covalently bonded to an oxygen atom that is covalently bonded to another alkyl group.

The term “ester” includes compounds and moieties that contain a carbon or a heteroatom bound to an oxygen atom that is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom that is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” include moieties with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

The term “electron-withdrawing group” “or electron-withdrawing atom” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (Σ) constant. This well known constant is described in many references, for instance, J. March, Advanced Organic Chemistry, McGraw Hill Book Company, New York, (1977 edition) pp. 251-259. The Hammett constant values are generally negative for electron donating groups (Σ[P]=−0.66 for NH₂) and positive for electron withdrawing groups (Σ[P]=0.78 for a nitro group), wherein Σ[P] indicates para substitution. Non-limiting examples of electron-withdrawing groups include nitro, acyl, formyl, sulfonyl, trifluoromethyl, cyano, chloride, carbonyl, thiocarbonyl, ester, imino, amido, carboxylic acid, sulfonic acid, sulfinic acid, sulfamic acid, phosphonic acid, boronic acid, sulfate ester, hydroxyl, mercapto, cyano, cyanate, thiocyanate, isocyanate, isothiocyanate, carbonate, nitrate and nitro groups and the like. Exemplary electron-withdrawing atoms include, but are not limited to, an oxygen atom, a nitrogen atom, a sulfur atom or a halogen atom, such as a fluorine, chlorine, bromine or iodine atom. It is to be understood that, unless otherwise indicated, reference herein to an acidic functional group also encompasses salts of that functional group in combination with a suitable cation.

Additionally, the phrase “any combination thereof” implies that any number of the listed functional groups and molecules may be combined to create a larger molecular architecture. For example, the terms “phenyl,” “carbonyl” (or “═O”), “—O—,” “—OH,” and C₁₋₆ (i.e., —CH₃ and —CH₂CH₂CH₂—) can be combined to form a 3-methoxy-4-propoxybenzoic acid substituent. It is to be understood that when combining functional groups and molecules to create a larger molecular architecture, hydrogens can be removed or added, as required to satisfy the valence of each atom.

The description of the disclosure herein should be construed in congruity with the laws and principals of chemical bonding. For example, it may be necessary to remove a hydrogen atom in order accommodate a substitutent at any given location. Furthermore, it is to be understood that definitions of the variables (i.e., “R groups”), as well as the bond locations of the generic formulae of the invention (e.g., formulas I or II), will be consistent with the laws of chemical bonding known in the art. It is also to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two-six bonds.

It will be noted that the structures of some of the compounds of this invention include asymmetric carbon atoms. It is to be understood accordingly that the isomers arising from such asymmetry (e.g., all enantiomers, stereoisomers, rotamers, tautomers, diastereomers, or racemates) are included within the scope of this invention. Such isomers can be obtained in substantially pure form by classical separation techniques and by stereochemically controlled synthesis. Furthermore, the structures and other compounds and moieties discussed in this application also include all tautomers thereof. Compounds described herein may be obtained through art recognized synthesis strategies.

It will also be noted that the substituents of some of the compounds of this invention include isomeric cyclic structures. It is to be understood accordingly that constitutional isomers of particular substituents are included within the scope of this invention, unless indicated otherwise. For example, the term “tetrazole” includes tetrazole, 2H-tetrazole, 3H-tetrazole, 4H-tetrazole and 5H-tetrazole.

### Use in Proliferative Diseases, Viral Infections, and Inflammation

The compounds of the present invention have valuable pharmacological properties and are useful in the treatment of diseases in a particular subject. In certain embodiments, the compounds of the invention can be used to treat proliferative diseases, such as Alzheimer's disease, viral infections, auto-immune diseases, fungal disease, cancer, psoriasis, vascular smooth cell proliferation associated with atherosclerosis, pulmonary fibrosis, arthritis glomerulonephritis, chronic inflammation, neurodegenerative disorders, such as Alzheimer's disease, and post-surgical stenosis and restenosis.

In other embodiments, the compounds of the invention can be used for the treatment of diseases associated with apoptosis, including, but not limited to, cancer, viral infections, autoimmune diseases and neurodegenerative disorders.

In still other embodiments, the compounds of the invention can be used to treat viral infections in a subject, wherein the viral infections are associated with, but are not limited to, HIV, human papilloma virus, herpes virus, poxvirus, Epstein-Barr virus, Sindbis virus and adenovirus.

In yet other embodiments, the compounds of the invention can be used to treat tumor angiogenesis and metastasis in a subject, as well as smooth muscle proliferation associated with atherosclerosis, postsurgical vascular stenosis and restenosis, and endometriosis

In certain embodiments, compounds of the invention are useful in the treatment of cancer. Examples of cancers that may be treated by the compounds of the invention include, but are not limited to, bladder, head and neck, breast, stomach, ovary, colon, lung, larynx, lymphatic system, hematopoetic system, genitourinary tract, gastrointestinal, ovarian, prostate, gastric, bone, small-cell lung, glioma, colorectal and pancreatic cancer, as well as cancers of the cervix, testis, esophagus, stomach, skin, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenocarcinoma, adenoma, adenocarcinoma, follicular carcinoma, undifferentiated carcinoma, papillary carcinoma, seminoma, melanoma, sarcoma, bladder carcinoma, liver carcinoma, kidney carcinoma, myeloid disorders, Hodgkin's, hairy cells, and leukemia. Other cancers that can be treated by the compounds of the invention include carcinomas, hematopoietic tumors of lymphoid lineage, hematopoietic tumors of myeloid lineage, tumors of mesenchymal origin, tumors of the central and peripheral nervous system, melanoma, seminoma, teratocarcinoma, osteosarcoma, xeroderoma pigmentosa, keratoctanthoma, thyroid follicular cancer and Kaposi's sarcoma.

In certain embodiments, the compounds of the invention can be used to modulate the level of cellular RNA and DNA synthesis in a patient in need thereof.

In other embodiments, the compounds of the invention can be used in the treatment of autoimmune diseases in a subject, wherein the autoimmune diseases include, but are not limited to, psoriasis, inflammation like rheumatoid arthritis, lupus, type 1 diabetes, diabetic nephropathy, multiple sclerosis, glomerulonephritis, chronic inflammation, and organ transplant rejections.

In other embodiments, the compounds of the invention can be used to treat diseases caused by a variety of infectious agents, including fungi, protozoan parasites such as Plasitiodium falciparum, and DNA and RNA viruses.

### Assays

The inhibition of protein kinase activity by the compounds of the invention may be measured using a number of assays available in the art. Examples of such assays are described in the Exemplification section below.

### Pharmaceutical Compositions

The language “effective amount” of the compound is that amount necessary or sufficient to treat or prevent a protein kinase-associated disorder, e.g. prevent the various morphological and somatic symptoms of a protein kinase-associated disorder, and/or a disease or condition described herein. In an example, an effective amount of the compound of the invention is the amount sufficient to treat a protein kinase-associated disorder in a subject. The effective amount can vary depending on such factors as the size and weight of the subject, the type of illness, or the particular compound of the invention. For example, the choice of the compound of the invention can affect what constitutes an “effective amount.” One of ordinary skill in the art would be able to study the factors contained herein and make the determination regarding the effective amount of the compounds of the invention without undue experimentation.

The regimen of administration can affect what constitutes an effective amount. The compound of the invention can be administered to the subject either prior to or after the onset of a protein kinase-associated disorder. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation.

Compounds of the invention may be used in the treatment of states, disorders or diseases as described herein, or for the manufacture of pharmaceutical compositions for use in the treatment of these diseases. Methods of use of compounds of the present invention in the treatment of these diseases, or pharmaceutical preparations having compounds of the present invention for the treatment of these diseases.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral and/or IV administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats a protein kinase-associated disorder.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

### Synthetic Procedure

Compounds of the present invention are prepared from commonly available compounds using procedures known to those skilled in the art, including any one or more of the following conditions without limitation:

Within the scope of this text, only a readily removable group that is not a constituent of the particular desired end product of the compounds of the present invention is designated a “protecting group,” unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively under physiological conditions (e.g., by enzymatic cleavage).

Acid addition salts of the compounds of the invention are most suitably formed from pharmaceutically acceptable acids, and include for example those formed with inorganic acids e.g. hydrochloric, hydrobromic, sulphuric or phosphoric acids and organic acids e.g. succinic, malaeic, acetic or fumaric acid. Other non-pharmaceutically acceptable salts e.g. oxalates can be used for example in the isolation of the compounds of the invention, for laboratory use, or for subsequent conversion to a pharmaceutically acceptable acid addition salt. Also included within the scope of the invention are solvates and hydrates of the invention.

The conversion of a given compound salt to a desired compound salt is achieved by applying standard techniques, in which an aqueous solution of the given salt is treated with a solution of base e.g. sodium carbonate or potassium hydroxide, to liberate the free base which is then extracted into an appropriate solvent, such as ether. The free base is then separated from the aqueous portion, dried, and treated with the requisite acid to give the desired salt.

In vivo hydrolyzable esters or amides of certain compounds of the invention can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds of the invention having a free carboxy group can be esterified using standard conditions which can include activation followed by treatment with the desired alcohol in the presence of a suitable base.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Particularly preferred salts are sodium, lysine and arginine salts of the compounds of the invention. Such salts can be formed by procedures well known and described in the art.

Other acids such as oxalic acid, which can not be considered pharmaceutically acceptable, can be useful in the preparation of salts useful as intermediates in obtaining a chemical compound of the invention and its pharmaceutically acceptable acid addition salt.

Metal salts of a chemical compound of the invention include alkali metal salts, such as the sodium salt of a chemical compound of the invention containing a carboxy group. Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable for example by means of fractional crystallisation, or by chromatography over optically active column materials.

Intermediates and final products can be worked up and/or purified according to standard methods, e.g., using chromatographic methods, distribution methods, (re-) crystallization, and the like.

### General Process Conditions

The following applies in general to all processes mentioned throughout this disclosure.

The process steps to synthesize the compounds of the invention can be carried out under reaction conditions that are known per se, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g., in the H⁺ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described in Science of Synthesis: Houben-Weyl Methods of Molecular Transformation. Georg Thieme Verlag, Stuttgart, Germany. 2005.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

### Prodrugs

This invention also encompasses pharmaceutical compositions containing, and methods of treating protein kinase-associated disorders through administering, pharmaceutically acceptable prodrugs of compounds of the compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

Any reference to a compound of the present invention is therefore to be understood as referring also to the corresponding pro-drugs of the compound of the present invention, as appropriate and expedient.

### Combinations

A compound of the present invention may also be used in combination with other agents, e.g., a chemotherapeutic or an additional protein kinase inhibitor that is or is not a compound of the invention, for treatment of a protein kinase-associated disorder in a subject.

By the term “combination” is meant either a fixed combination in one dosage unit form, or a kit of parts for the combined administration where a compound of the present invention and a combination partner may be administered independently at the same time or separately within time intervals that especially allow that the combination partners show a cooperative, e.g., synergistic, effect, or any combination thereof.

The compounds of the invention may be administered, simultaneously or sequentially, with an antiinflammatory, antiproliferative, chemotherapeutic agent, immunosuppressant, anti-cancer, cytotoxic agent or kinase inhibitor other than a compound of the Formula I or salt thereof. Further examples of agents that may be administered in combination with the compounds of the invention include, but are not limited to, a PTK inhibitor, cyclosporin A, CTLA4-Ig, antibodies selected from anti-ICAM-3, anti-IL-2 receptor, anti-CD45RB, anti-CD2, e.g., CVT-313, anti-CD3, anti-CD4, anti-CD80, anti-CD86, and monoclonal antibody OKT3, agents blocking the interaction between CD40 and gp39, fusion proteins constructed from CD40 and gp39, inhibitors of NF-kappa B function, non-steroidal antiinflammatory drugs, steroids, gold compounds, antiproliferative agents, FK506, mycophenolate mofetil, cytotoxic drugs, TNF-α inhibitors, anti-TNF antibodies or soluble TNF receptor, TNFalpha, TRAIL, HDAC inhibitors, gleevec, and other inhibitors of signal transduction pathways involved in cell proliferation, inhibitors of cellular responses to hypoxia, rapamycin, leflunimide, cyclooxygenase-2 inhibitors, paclitaxel, cisplatin, carboplatin, doxorubicin, caminomycin, daunorubicin, aminopterin, methotrexate, methopterin, mitomycin C, ecteinascidin 743, porfiromycin, 5-fluorouracil, 6-mercaptopurine, gemcitabine, cytosine arabinoside, podophyllotoxin, etoposide, etoposide phosphate, teniposide, melphalan, vinblastine, vincristine, leurosidine, epothilone, vindesine, leurosine, or derivatives thereof.

Further examples of agents that may be administered in combination with the compounds of the invention include, but are not limited to, anti-proliferating agents selected from the group consisting of: altretamine, busulfan, chlorambucil, cyclophosphamide, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, prednisone, bleomycin, dactinomycin, daunorubicin, doxirubicin, idarubicin, mitoxantrone, losoxantrone, mitomycin-c, plicamycin, paclitaxel, docetaxel, CPT-11, epothilones, topotecan, irinotecan, 9-amino camptothecan, 9-nitro camptothecan, GS-211, etoposide, teniposide, vinblastine, vincristine, vinorelbine, procarbazine, asparaginase, pegaspargase, methoxtrexate, octreotide, estramustine, and hydroxyurea.

The compound of the invention and any additional agent may be formulated in separate dosage forms. Alternatively, to decrease the number of dosage forms administered to a patient, the compound of the invention and any additional agent may be formulated together in any combination. For example, the compound of the invention inhibitor may be formulated in one dosage form and the additional agent may be formulated together in another dosage form. Any separate dosage forms may be administered at the same time or different times.

Alternatively, a composition of this invention comprises an additional agent as described herein. Each component may be present in individual compositions, combination compositions, or in a single composition.

### Exemplification of the Invention

The invention is further illustrated by the following examples, which should not be construed as further limiting. The practice of the present invention will employ, unless otherwise indicated, conventional techniques of cell biology, cell culture, molecular biology, transgenic biology, microbiology and immunology, which are within the skill of the art.

### General Synthesis Methods

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

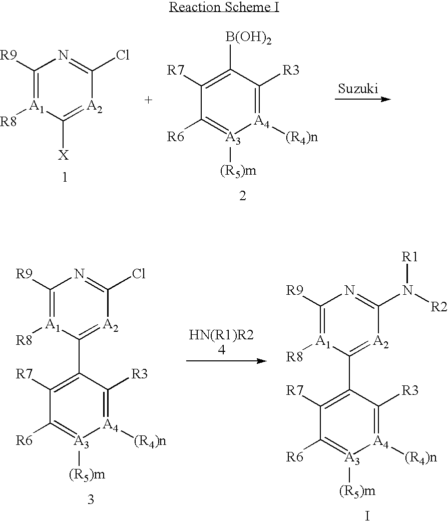

Compounds of Formula I, including substituted or unsubstituted pyridyl-pyridines, pyridyl-pyrimidines and pyridyl-triazines, can be prepared according to the method generally described in Reaction Scheme I. A compound of formula 3 can be synthesized utilizing Suzuki coupling methodology on a compound of formula 1 (where X is a halide such as iodide, bromide or chloride), with an aryl borinate of formula 2 in the presence of a suitable catalyst (for example, Pd(PPh₃)₄, (Ph₃P)₂PdCl₂ and the like), a suitable base (for example, Na₂CO₃, K₂CO₃, NaHCO₃, and the like) and a suitable solvent (for example, DME, dioxane and the like). Similarly, other known aryl coupling methods, such as the use of stannanes, zincates and copper coupling techniques are also suitable to prepare compounds of formula 3. A compound of formula I can be synthesized by reacting a compound of formula 3 with a compound of formula 4 in the presence of a suitable solvent (for example, sec-butanol, dioxane and the like) and a suitable catalyst (for example, p-toluenesulfonic acid monohydrate, and the like). The reaction proceeds in a temperature range of 60° C. to about 130° C. and can take up to about 24 hours to complete. Alternatively, compounds of formula I can be synthesized by reacting a compound of formula 3 with a compound of formula 4 in the presence of a suitable solvent (for example dioxane, and the like) and a suitable catalyst (for example, palladium acetate, and the like) and a suitable ligand (for example XantPhos, BINAP, and the like) and a suitable base (for example cesium carbonate, and the like). The reaction proceeds in a temperature range of 60° C. to about 130° C. and can take up to about 24 hours to complete.

Compounds of formula 7 can be prepared as in the following Reaction Scheme II:

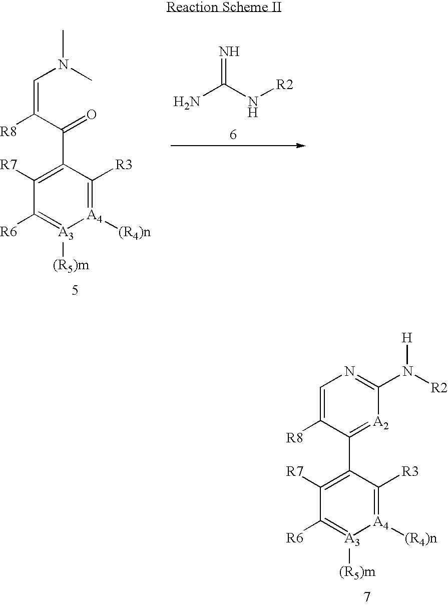

A compound of formula 7 can be synthesized by reacting a compound of formula 5 with a compound of formula 6 in the presence of a suitable solvent (for example, sec-butanol, and the like). The reaction proceeds in a temperature range of 20° C. to about 100° C. and can take up to about 24 hours to complete.

Detailed examples of the synthesis of compounds of Formula I can be found in the examples, infra.

### Example 1

### N-(3-{4-[2-(3-Chloro-phenylamino)-pyrimidin-4-yl]-3-methyl-pyridin-2-ylamino}-propyl)-acetamide (6)

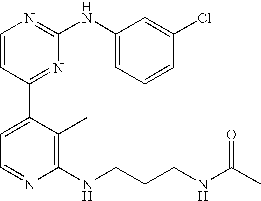

Prepared as in Reaction Scheme III:

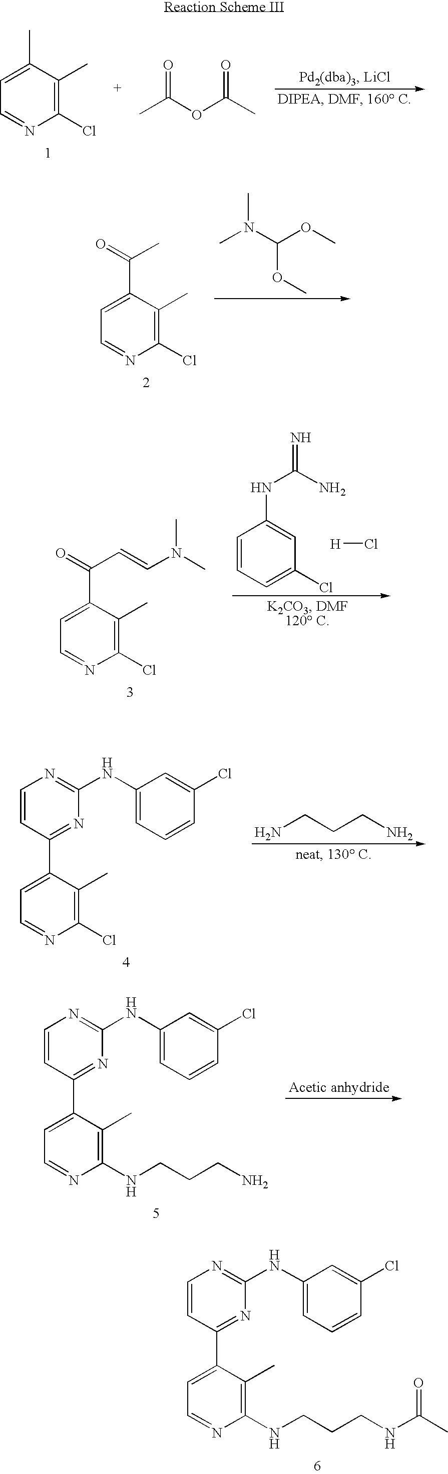

### 1-(2-Chloro-3-methyl-pyridin-4-yl)-ethanone (2)

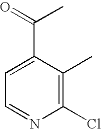

A suspension of 2-chloro-4-iodo-3-methyl-pyridine (1.25 g, 4.88 mmol), acetic anhydride (2.31 mL, 24.5 mmol), lithium chloride (1.03 g, 24.2 mmol), Pd₂(dba)₃ (90 mg, 0.1 mmol), Hüinig's base (1.71 mL, 9.8 mmol) in DMF is heated at 160° C. in the microwave for 20 min. The reaction is partitioned between EtOAc and saturated NaHCO₃(aq). The organics layer was collected and washed with brine, dried over Na₂SO₄ and stripped down in vacuo. The crude product is purified by silica column chromatography eluting with 20% EtOAc: Hexanes giving 1-(2-chloro-3-methyl-pyridin-4-yl)-ethanone (408 mg, 2.41 mmol) as a clear oil. ¹H NMR (400 MHz, CDCl₃) δ 2.46 (s, 3H), 2.59 (s, 3H), 7.28 (d, 1H), 8.36 (d, 1H); MS m/z 170.2 (M+1).

### (2-Chloro-3-methylpyridin-4-yl)-3-dimethylamino-propenone (3)

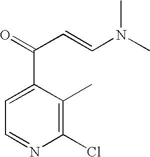

A solution of (2-chloro-3-methyl-pyridin-4-yl)-3-dimethylamino-propenone (1.63 g, 9.51 mmol) in DMF-DMA (3 mL) is heated at 80° C. for 2 hours. The reaction is concentrated in vacuo and purified by silica column eluting with methylene chloride and methanol giving (2-chloro-3-methylpyridin-4-yl)-3-dimethylamino-propenone (1.3 g, 5.79 mmol) as an orange solid. ¹H NMR (400 MHz, CDCl₃) δ 2.40 (br.s, 3H), 2.93 (s, 3H), 3.18 (br.s, 3H), 5.32 (br.s, 1H), 7.14 (br.s, 1H), 7.28 (m, 1H), 8.26 (d, J=4.6 Hz, 1H); MS m/z 225.2 (M+1).

### 4-(2-Chloro-3-methyl-pyridin-4-yl)-pyrimidin-2-yl]-(3-chloro-phenyl)-amine (4)

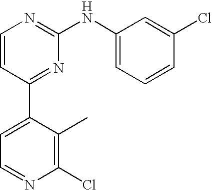

A solution of (2-chloro-3-methylpyridin-4-yl)-3-dimethylamino-propenone, (1.3 g, 5.73 mmol), N-(3-chloro-phenyl)-guanidine.HCl (1.79 g, 8.6 mmol) in DMF over potassium carbonate (2.4 g, 17.2 mmol) is heated at 120° C. overnight. The reaction is partitioned between ethyl acetate and water and extracted. The organics are washed with brine, dried (Na₂SO₄) and stripped down in vacuo. The crude product is purified by silica column eluting with ethyl acetate and hexanes giving 4-(2-chloro-3-methylpyridin-4-yl)-pyrimidin-2-yl]-(3-chloro-phenyl)amine (1.2 g, 3.62 mmol). ¹H NMR (400 MHz, CDCl₃) δ 2.34 (s, 3H), 6.75 (d, J=5.1 Hz, 1H), 6.94 (s, 1H), 7.11-7.16 (m, 1H), 7.29 (m, 1H), 7.39 (br.s, 1H), 7.72 (m, 1H), 8.24 (d, J=5.1 Hz, 1H), 8.44 (d, J=5.1 Hz, 1H); MS m/z 331.2 (M+1).

### N-{4-[2-(3-Chloro-phenylamino)-pyrimidin-4-yl]-3-methyl-pyridin-2-yl}propane-1,3-diamine (5)

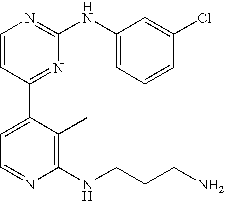

A solution of 4-(2-chloro-3-methylpyridin-4-yl)-pyrimidin-2-yl]-(3-chloro-phenyl)amine (1.2 g, 3.59 mmol) in 1,3-propanediamine is heated at 140° C. overnight. The reaction is partitioned between ethyl acetate and water and extracted. The organics were dried over sodium sulfate and stripped down in vacuo. The crude product was purified by silica column eluting with methylene chloride and methanol giving N-{4-[2-(3-Chloro-phenylamino)-pyrimidin-4-yl]-3-methyl-pyridin-2-yl}-propane-1,3-diamine (1.02 g, 2.77 mmol) as a clear oil. ¹H NMR (400 MHz, MeOD) δ 2.14 (q, J=7.1 Hz, 2H), 2.45 (s, 3H), 3.07 (t, J=6.2 Hz, 2H), 3.85 (t, J=6.82 Hz, 2H), 6.93 (d, 1H), 7.22 (d, J=5.1 Hz, 2H), 7.27 (d, J=8.1 Hz, 1H), 7.54 (t, 1H), 7.86 (d, 1H), 8.25 (d, J=5.1 Hz, 2H), 8.29 (s, 1H), 8.84 (d, 1H); MS m/z 369.2 (M+1).

### N-(3-{4-[2-(3-Chloro-phenylamino)-pyrimidin-4-yl]-3-methyl-pyridin-2-ylamino}-propyl)-acetamide (6)

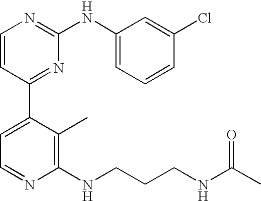

To a solution of N-{4-[2-(3-chloro-phenylamino)-pyrimidin-4-yl]-3-methyl-pyridin-2-yl}-propane-1,3-diamine (0.98 g, 2.63 mmol) in methylene chloride was added acetic anhydride (0.25 mL, 2.63 mmol) dropwise. After 15 minutes the reaction was stripped down in vacuo and placed in a vacuum oven at 40° C. for 3 days giving N-(3-{4-[2-(3-chloro-phenylamino)-pyrimidin-4-yl]-3-methyl-pyridin-2-ylamino}-propyl)-acetamide (1.06 g, 2.59 mmol) as a yellow solid. ¹H NMR 400 MHz (MeOD) δ 1.81 (quintet, J=6.6 Hz, 2H), 1.96 (s, 3H), 2.15 (s, 3H), 3.28 (t, J=6.6 Hz, 2H), 3.49 (t, J=6.6 Hz, 2H), 6.62 (d, J=5.1 Hz, 1H), 6.90 (d, J=5.1 Hz, 1H), 6.95 (d, J=7.6 Hz, 1H), 7.23 (t, J=8.1 Hz, 1H), 7.55 (d, J=8.1 Hz, 1H), 7.94 (d, J=5.1 Hz, 1H), 7.98 (s, 1H), 8.52 (d, J=5.1 Hz, 1H); MS m/z 411.2 (M+1).

The procedures described in the above examples, using appropriate starting materials, can be used to prepare the compounds of Formulas I, II and III. The spectroscopic data shown in Table D are for a selection of the compounds of the invention.

TABLE D

| Compound | | |
|---|---|---|
| no. | ¹H NMR 400 MHz | MS (m/z) |
| 1 | (MeOD) δ 6.97 (d, J = 8.1 Hz, 1H), 7.24 (t, J = 8.1 Hz, | 298.3 (M + 1) |
| | 1H), 7.32 (d, J = 5.6 Hz, 1H), 7.50 (dd, J = 8.1, | |
| | 2.0 Hz, 1H), 7.61 (d, J = 5.6 Hz, 1H), 7.76 (d, J = 5.6 Hz, | |
| | 1H), 7.85 (t, J = 1.8 Hz, 1H), 8.11 (s, 1H), | |
| | 8.51 (d, J = 5.6 Hz, 1H) | |
| 2 | (CDCl₃) δ 2.34 (s, 3H), 6.75 (d, J = 5.1 Hz, 1H), | 331.2 (M + 1) |
| | 6.94 (s, 1H), 7.13 (m, 1H), 7.29 (m, 1H), 7.39 (br.s, | |
| | 1H), 7.72 (m, 1H), 8.24 (d, J = 5.1 Hz, 1H), | |
| | 8.44 (d, J = 5.1 Hz, 1H) | |
| 3 | (CDCl₃) δ 2.15 (s, 3H), 2.54 (s, 3H), 2.99 (t, J = 5.3 Hz, | 369.2 (M + 1) |
| | 2H) 3.68 (dd, J = 5.6, 5.6 Hz, 2H), 5.08 (brs, | |
| | 1H), 6.63 (d, J = 5.6 Hz, 1H), 6.83 (d, J = 5.1 Hz, | |
| | 1H), 7.00 (ddd, J = 8.1, 1.0, 1.0 Hz, 1H), | |
| | 7.20-7.29 (m, 2H), 7.41 (ddd, J = 8.1, 1.0, 1.0 Hz, 1H), | |
| | 7.86 (brs, 1H), 8.07 (d, J = 5.6 Hz, 1H), 8.51 (d, J = 5.1 Hz, | |
| | 1H) | |
| 4 | (MeOD) δ 2.14 (quintet, J = 7.1 Hz, 2H), 2.45 (s, | 369.2 (M + 1) |
| | 3H), 3.07 (t, J = 6.2 Hz, 2H), 3.85 (t, J = 6.3 Hz, | |
| | 2H), 6.93 (d, 1H), 7.22 (d, J = 5.1 Hz, 2H), 7.27 (d, | |
| | J = 8.1 Hz, 1H), 7.54 (t, 1H), 7.86 (d, 1H), 8.25 (d, | |
| | J = 5.1 Hz, 2H), 8.29 (s, 1H), 8.84 (d, 1H) | |
| 5 | (MeOD) δ 1.12 (t, J = 7.6 Hz, 3H), 1.84 (quintet, J = 6.8 Hz, | 383.3 (M + 1) |
| | 2H), 2.58 (q, J = 7.6 Hz, 2H), 2.76 (t, J = 6.8 Hz, | |
| | 2H), 3.55 (t, J = 6.6 Hz, 2H), 6.54 (d, J = 5.6 Hz, | |
| | 1H), 6.87 (d, J = 5.1 Hz, 1H), 6.98 (dd, J = 8.1, | |
| | 2.0 Hz, 1H), 7.24 (t, J = 8.1 Hz, 1H), 7.57 (dd, | |
| | J = 8.1, 2.0 Hz, 1H), 7.89-7.94 (m, 2H), 8.53 (d, J = 5.1 Hz, | |
| | 1H) | |
| 6 | (MeOD) δ 1.81 (quintet, J = 6.6 Hz, 2H), 1.96 (s, | 411.2 (M + 1) |
| | 3H), 2.15 (s, 3H), 3.28 (t, J = 6.6 Hz, 2H), 3.49 (t, J = 6.6 Hz, | |
| | 2H), 6.62 (d, J = 5.1 Hz, 1H), 6.90 (d, J = 5.1 Hz, | |
| | 1H), 6.95 (d, J = 7.6 Hz, 1H), 7.23 (t, J = 8.1 Hz, | |
| | 1H), 7.55 (d, J = 8.1 Hz, 1H), 7.94 (d, J = 5.1 Hz, | |
| | 1H), 7.98 (s, 1H), 8.52 (d, J = 5.1 Hz, 1H); | |
| 7 | (MeOD) δ 1.09 (t, J = 7.6 Hz, 3H), 1.78 (quintet, J = 6.7 Hz, | 425.2 (M + 1) |
| | 2H), 1.93 (s, 3H), 2.55 (q, J = 7.6 Hz, | |
| | 2H), 3.21-3.30 (m, 2H), 3.46 (t, J = 6.6 Hz, 2H), | |
| | 3.60 (br.s, 1H), 6.51 (d, J = 5.6 Hz, 1H), 6.83 (d, J = 4.6 Hz, | |
| | 1H), 6.93 (dd, J = 7.6, 1.5 Hz, 1H), | |
| | 7.20 (t, J = 8.1 Hz, 1H), 7.53 (dd, J = 8.6, 2.0 Hz, 1H), | |
| | 7.87-7.91 (m, 2H), 8.49 (d, J = 5.1 Hz, 1H) | |
| 8 | (MeOD) δ 1.91 (quintet, J = 6.8 Hz, 2H), 2.02 (s, | 383.1 (M + 1) |
| | 3H), 3.33 (t, J = 7.1 Hz, 2H), 3.43 (t, J = 6.8 Hz, | |
| | 2H) 3.49 (s, 3H), 6.52 (d, J = 5.1 Hz, 1H), 6.78 (d, | |
| | J = 5.1 Hz, 1H), 7.19 (d, J = 7.1 Hz, 1H), 7.24 (d, J = 8.1 Hz, | |
| | 1H), 7.32 (d, J = 8.1 Hz, 1H), 7.37 (t, J = 2.0 Hz, | |
| | 1H), 7.85 (d, J = 5.1 Hz, 1H), 8.35 (d, J = 5.2 Hz, | |
| | 1H) | |
| 9 | (MeOD) δ 1.79 (quintet, J =, 7.3 Hz, 2H), 2.30 (s, | 397.3 (M + 1) |
| | 3H), 2.31 (s, 3H), 2.55 (t, J = 7.6 Hz, 2H), 2.84 (s, | |
| | 3H), 3.23 (t, J = 7.1 Hz, 2H), 6.93 (dd, J = 8.3, 1.8 Hz, | |
| | 1H), 6.95 (d, J = 5.1 Hz, 1H), 7.03 (d, J = 5.1 Hz, | |
| | 1H), 7.20 (t, J = 8.1 Hz, 1H), 7.46 (dd, J = 7.8, | |
| | 1.8 Hz, 1H), 8.03 (t, J = 2.0 Hz, 1H), 8.13 (d, J = 5.6 Hz, | |
| | 1H), 8.52 (d, J = 5.1 Hz, 1H) | |
| 10 | Obtained as a mixture of rotamers: (MeOD) δ | 439.2 (M + 1) |
| | 1.77-1.91 (m, 2H), 1.98 (s, 1.5H), 2.03 (s, 1.5H), 2.31 (s, | |
| | 1.5H), 2.33 (s, 1.5H), 2.85 (s, 3H), 2.85 (s, 1.5H), | |
| | 2.99 (s, 1.5H), 3.20 (t, J = 7.3 Hz, 1H), 3.25 (t, J = 6.6 Hz, | |
| | 1H), 3.35-3.41 (m, 2H), 6.94 (dd, J = 7.8, | |
| | 1.3 Hz, 1H), 6.97 (d, J = 5.1 Hz, 1H), 7.05 (d, J = 5.1 Hz, | |
| | 0.5H), 7.07 (d, J = 5.1 Hz, 0.5H), 7.21 (t, J = 8.1 Hz, | |
| | 1H), 7.48 (m, 1H), 8.03 (m, 1H), 8.15 (d, | |
| | J = 5.1 Hz, 1H), 8.18 (d, J = 5.1 Hz, 1H), 8.53 (d, J = 5.05 Hz, | |
| | 1H); | |
| 11 | (MeOD) δ 1.79 (quintet, J = 7.1 Hz, 2H), 2.23 (s, | 411.2 (M + 1) |
| | 3H), 2.36 (s, 3H), 2.60 (t, J = 7.1 Hz, 2H), 2.81 (s, | |
| | 3H), 3.21 (t, J = 7.1 Hz, 2H), 3.53 (s, 3H), 6.88 (d, | |
| | J = 5.1 Hz, 1H), 6.99 (d, J = 5.1 Hz, 1H), 7.21 (d, J = 8.1 Hz, | |
| | 1H), 7.27 (d, J = 7.1 Hz, 1H), 7.36 (t, J = 8.08 Hz, | |
| | 1H), 7.41 (t, J = 2.0 Hz, 1H), 8.11 (d, J = 5.6 Hz, | |
| | 1H), 8.41 (d, J = 5.0 Hz, 1H) | |
| 12 | (MeOD) δ 1.75-1.88 (m, 2H), 1.95 (s, 1.5H), | 453.2 (M + 1) |
| | 2.03 (s, 1.5H), 2.24 (s, 1.5H), 2.24 (s, 1.5H), 2.80 (s, | |
| | 3H), 2.83 (s, 1.5H), 2.97 (s, 1.5H), 3.15 (t, J = 7.0 Hz, | |
| | 1H), 3.21 (t, J = 6.6 Hz, 1H), 3.31-3.39 (m, | |
| | 2H), 3.53 (s, 3H), 6.88 (dd, J = 5.1, 1.0 Hz, 1H), | |
| | 6.98 (d, J = 5.1 Hz, 0.5H), 7.02 (d, J = 5.1 Hz, | |
| | 0.5H), 7.21 (d, J = 8.1 Hz, 1H), 7.27 (d, J = 8.1 Hz, | |
| | 1H), 7.36 (t, J = 8.0, 1H), 7.41 (t, J = 1.77 Hz, 1H), | |
| | 8.11 (d, J = 5.1 Hz, 0.5H), 8.14 (d, J = 5.1 Hz, | |
| | 0.5H), 8.41 (d, J = 5.05 Hz, 0.5H), 8.42 (d, J = 5.05 Hz, | |
| | 0.5H) | |
| 13 | | 425.2 (M + 1) |
| 14 | (MeOD) δ 1.81 (quintet, J = 6.6 Hz, 2H), 1.96 (s, | 406.4 (M + 1) |
| | 3H), 2.14 (s, 3H), 3.27 (t, J = 6.6 Hz, 2H), 3.49 (t, J = 6.7 Hz, | |
| | 2H), 3.78 (s, 2H), 6.62 (d, J = 5.6 Hz, | |
| | 1H), 6.85 (d, J = 5.1 Hz, 1H), 6.99 (d, J = 7.6 Hz, | |
| | 1H), 7.26 (t, J = 7.6 Hz, 1H), 7.61-7.66 (m, 2H), | |
| | 7.94 (d, J = 5.6 Hz, 1H), 8.48 (d, J = 5.1 Hz, 1H) | |
| 15 | (MeOD) δ 1.79 (quintet, J = 6.8 Hz, 2H), 1.91 (s, | 448.4 (M + 1) |
| | 3H), 1.93 (s, 3H), 2.11 (s, 3H), 3.25 (t J = 7.0 Hz, | |
| | 2H), 3.46 (t, J = 6.8 Hz, 2H), 4.31 (s, 2H), 6.59 (d, | |
| | J = 5.6 Hz, 1H), 6.82 (d, J = 5.1 Hz, 1H), 6.89 (d, J = 7.6 Hz, | |
| | 1H), 7.21 (t, J = 7.8 Hz, 1H), 7.56 (d, J = 8.1 Hz, | |
| | 1H), 7.65 (br.s, 1H), 7.91 (d, J = 5.1 Hz, | |
| | 1H), 8.45 (d, J = 5.1 Hz, 1H) | |
| 16 | | 411.2 (M + 1) |
| 17 | (MeOD) δ 1.83 (quintet, J = 6.3 Hz, 2H), 2.11 (s, | 370.3 (M + 1) |
| | 3H), 3.54 (t, J = 6.6 Hz, 2H), 3.65 (t, J = 6.1 Hz, | |
| | 2H), 6.59 (d, J = 5.6 Hz, 1H), 6.88 (d, J = 5.1 Hz, | |
| | 1H), 6.93 (dd, J = 7.6, 2.0 Hz, 1H), 7.20 (t, J = 8.1 Hz, | |
| | 1H), 7.52 (dd, J = 8.1, 2.0 Hz, 1H), 7.90 (d, J = 5.1 Hz, | |
| | 1H), 7.95 (t, J = 2.02 Hz, 1H), 8.50 (d, J = 5.1 Hz, | |
| | 1H) | |
| 18 | (CDCl₃) δ 1.98 (quintet, J = 5.8 Hz, 2H), 2.13 (s, | 384.2 (M + 1) |
| | 3H), 3.38 (s, 3H), 3.60 (t, J = 5.3 Hz, 2H), | |
| | 3.70 (brs, 2H), 6.63 (d, J = 5.1 Hz, 1H), 6.84 (d, J = 5.1 Hz, | |
| | 1H), 7.00 (d, J = 8.1 Hz, 1H), 7.22 (t, J = 8.1 Hz, | |
| | 1H), 7.40 (dd, J = 8.1, 2.0 Hz, 1H), 7.87 (t, J = 2.0 Hz, | |
| | 1H), 8.10 (d, J = 5.6 Hz, 1H), 8.52 (d, J = 5.1 Hz, | |
| | 1H) | |
| 19 | (MeOD) δ 1.97 (quintet, J = 6.8 Hz, 2H), 2.01 (s, | 412.1 (M + 1) |
| | 3H), 2.12 (s, 3H), 3.52 (t, J = 6.8 Hz, 2H), 4.17 (t, J = 6.3 Hz, | |
| | 2H), 6.60 (d, J = 5.6 Hz, 1H), 6.88 (d, J = 5.1 Hz, | |
| | 1H), 6.93 (dd, J = 8.1, 2.0 Hz, 1H), 7.20 (t, | |
| | J = 8.1 Hz, 1H), 7.52 (dd, J = 8.1, 2.0 Hz, 1H), | |
| | 7.92 (d, J = 5.6 Hz, 1H), 7.95 (t, J = 2.0 Hz, 1H), | |
| | 8.50 (d, J = 5.1 Hz, 1H) | |
| 20 | (MeOD) δ 2.11 (s, 3H), 2.63 (t, J = 6.6 Hz, 2H), | 384.1 (M + 1) |
| | 3.69 (t, J = 6.6 Hz, 2H), 6.67 (d, J = 5.6 Hz, 1H), | |
| | 6.88 (d, J = 5.1 Hz, 1H), 6.91 (d, J = 7.6 Hz, 1H), | |
| | 7.19 (t, J = 8.1 Hz, 1H), 7.51 (dd, J = 8.1, 2.0 Hz, | |
| | 1H), 7.89-7.95 (m, 2H), 8.50 (d, J = 4.6 Hz, 1H) | |
| 21 | (CDCl₃) δ 2.15 (s, 3H), 4.69 (brs, 1H), 4.77 (d, J = 4.6 Hz, | 402.1 (M + 1) |
| | 2H), 6.68 (d, J = 6.68 Hz, 1H), 6.85 (d, J = 5.1 Hz, | |
| | 1H), 7.00 (d, J = 8.1 Hz, 1H), 7.22 (t, J = 8.1 Hz, | |
| | 1H), 7.27-7.31 (m, 2H), 7.36 (t, J = 7.3 Hz, | |
| | 2H), 7.41 (d, J = 7.1 Hz, 2H), 7.86 (t, J = 2.1 Hz, | |
| | 1H), 8.14 (d, J = 5.6 Hz, 1H), 8.52 (d, J = 5.1 Hz, | |
| | 1H) | |
| 22 | (CDCl₃) δ 1.95 (brs, 3H), 2.17 (s, 3H) 2.65 (brs, | 439.2 (M + 1) |
| | 5H), 3.63 (q, J = 5.6 Hz, 2H), 3.82 (brs, 4H), | |
| | 6.59 (d, J = 5.1 Hz, 1H), 6.84 (dd, J = 5.1, 1.0 Hz, 1H), | |
| | 7.00 (d, J = 7.58 Hz, 1H), 7.22 (t, J = 8.1 Hz, 1H), | |
| | 7.40 (d, J = 8.1 Hz, 1H), 7.89 (s, 1H), 8.08 (d, J = 5.1 Hz, | |
| | 1H), 8.51 (dd, J = 5.1, 1.0 Hz, 1H) | |
| 23 | (MeOD) δ 2.10-2.20 (m, 5H), 3.47 (t, J = 6.6 Hz, | 420.2 (M + 1) |
| | 2H), 4.14 (t, J = 6.6 Hz, 2H), 6.64 (d, J = 5.6 Hz, | |
| | 1H), 6.90 (d, J = 5.1 Hz, 1H), 6.93-6.98 (m, 2H), | |
| | 7.15 (s, 1H), 7.22 (t, J = 8.3 Hz, 1H), 7.54 (d, J = 8.1 Hz, | |
| | 1H), 7.67 (s, 1H), 7.93-7.99 (m, 2H), | |
| | 8.52 (d, J = 5.1 Hz, 1H) | |
| 24 | (CDCl₃) δ 2.02-2.12 (m, 3H), 2.23 (s, 3H), 2.44 (t, | 437.2 (M + 1) |
| | J = 8.1 Hz, 2H), 3.38-3.45 (m, 5H), 3.61 (brs, 2H), | |
| | 6.62 (d, J = 4.6 Hz, 1H), 6.83 (d, J = 4.6 Hz, | |
| | 1H), 7.00 (d, J = 7.6 Hz, 1H), 7.22 (t, J = 8.6 Hz, | |
| | 1H), 7.43 (d, J = 8.1 Hz, 1H), 7.84 (s, 1H), 8.06 (d, | |
| | J = 5.6 Hz, 1H), 8.52 (d, J = 4.6 Hz, 1H) | |
| 25 | (MeOD) δ 1.79 (quintet, J = 6.6 Hz, 2H), 1.93 (s, | 422.3 (M + 1) |
| | 3H), 2.15 (s, 3H), 3.22-3.30 (m, 2H), 3.47 (t, J = 6.6 Hz, | |
| | 2H), 6.63 (dd, J = 5.1, 2.5 Hz, 1H), | |
| | 6.95 (dd, J = 5.1, 2.5 Hz, 1H), 7.46 (td, J = 8.2, 2.3 Hz, | |
| | 1H), 7.79 (d, J = 8.1 Hz, 1H), 7.90-7.98 (m, 2H), | |
| | 8.56 (dd, J = 5.0, 2.5 Hz, 1H), 8.92 (d, J = 2.0 Hz, | |
| | 1H) | |
| 26 | | 587.3 (M + 1) |
| 27 | (MeOD) δ 2.04 (quintet, J = 6.8 Hz, 2H), 2.22 (s, | 474.1 (M + 1) |
| | 3H), 3.26 (t, J = 6.6 Hz, 2H), 3.60 (t, J = 6.8 Hz, | |
| | 2H), 3.90 (s, 2H), 6.74 (d, J = 8.1 Hz, 2H), | |
| | 6.89-6.95 (m, 3H), 7.14-7.20 (m, 3H), 7.45 (d, J = 8.1 Hz, | |
| | 1H), 7.75 (d, J = 6.57 Hz, 1H), 7.90 (br.s, 1H), | |
| | 8.56 (d, J = 5.1 Hz, 1H) | |
| 28 | (MeOD) δ 1.88-1.96 (m, 5H), 2.09 (s, 3H), 3.18 (t, | 516.3 (M + 1) |
| | J = 6.8 Hz, 2H), 3.54 (t, J = 6.8 Hz, 2H), 4.17 (s, | |
| | 2H), 6.58-6.62 (m, 3H), 6.88 (d, J = 5.1 Hz, 1H), | |
| | 6.93 (dd, J = 7.6, 2.02 Hz, 1H), 7.02 (d, J = 8.6 Hz, | |
| | 2H), 7.20 (t, J = 8.1 Hz, 1H), 7.52 (dd, J = 8.1, 2.0 Hz, | |
| | 1H), 7.92 (d, J = 5.6 Hz, 1H), 7.95 (t, J = 2.0 Hz, | |
| | 1H), 8.50 (d, J = 5.1 Hz, 1H) | |
| 29 | (MeOD) δ 1.88-1.91 (2s(rotamers), 3H), 3.16 (t, J = 6.8 Hz, | 549.3 (M + 1) |
| | 2H), 3.49 (t, J = 6.8 Hz, 2H), 4.4 (brs, 1H), | |
| | 6.53 (d, J = 5.0 Hz, 1H), 6.79-6.87 (m, 3H), | |
| | 6.92 (m, 1H), 7.11-7.16 (m, 3H), 7.37-7.50 (m, 3H), | |
| | 7.65 (d, J = 7.6 Hz, 1H), 7.84 (d, J = 5.6 Hz, 1H), | |
| | 7.88 (m, 1H), 8.43 (d, J = 5.1 Hz, 1H) | |
| 30 | (MeOD) δ 1.91 (d, J = 6.6 Hz, 2H), 1.91 (s, 3H), | 549.3 (M + 1) |
| | 3.23 (m, 2H), 3.50 (m, 2H), 6.53-6.55 (m, 3H), | |
| | 6.80 (d, J = 5.1 Hz, 1H), 6.85 (m, 1H), 7.12 (t, J, 1H), | |
| | 7.38 (m, 2H), 7.44 (d, J = 7.6 Hz, 2H), 7.52 (m, | |
| | 4H), 7.85 (m, 1H), 7.88 (m, 1H), 8.43 (d, J = 5.0 Hz, | |
| | 1H), 1H) | |
| 31 | | 369.3 (M + 1) |
| 32 | | 411.2 (M + 1) |
| 33 | | 369.2 (M + 1) |
| 34 | | 411.2 (M + 1) |
| 35 | | 381.1 (M + 1) |
| 36 | | 423.3 (M + 1) |
| 37 | | 381.3 (M + 1) |
| 38 | | 423.3 (M + 1) |
| 39 | | 369.2 (M + 1) |
| 40 | | 411.2 (M + 1) |
| 41 | | 369.2 (M + 1) |
| 42 | | 411.2 (M + 1) |

### Example 2

### N-(3-{3-[2-(3-Chloro-phenylamino)-pyrimidin-4-yl]-pyridin-2-ylamino}-propyl)-acetamide (65)

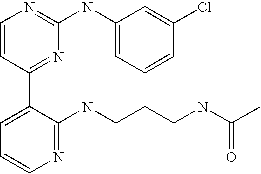

Part a A mixture of (2-chloro-3-pyridyl)boronic acid (1.49 g, 10.0 mmol), 2,4-dichloropyrimidine (1.57 mg, 10.0 mmol), sodium carbonate (2.52 g, 30.0 mmol) and Pd(PPh₃)₄ (1.12 g, 1.0 mmol) in 1,4-dioxane (20 mL) and water (5 mL) is degassed (nitrogen) and heated at 95° C. for 14 h. On cooling the solvent is removed in vacuo and the resulting crude material is purified by flash column chromatography eluting with ethyl acetate/heptane to afford 2-chloro-4-(2-chloro-pyridin-3-yl)pyrimidine.

Part b To 2-chloro-4-(2-chloro-pyridin-3-yl)pyrimidine (226 mg, 1.0 mmol) and para-toluenesulfonic acid monohydrate (210 mg, 1.1 mmol) in 1,4-dioxane (4.5 mL) is added 3-chloroaniline (105 μL, 1.0 mmol). The resultant reaction mixture is heated in a sealed tube at 110° C. for 1.5 h. On cooling the solvent is removed in vacuo and the resulting crude material is purified by flash column chromatography eluting with methanol/methylene chloride to afford (3-chloro-phenyl)-[4-(2-chloro-pyridin-3-yl)-pyrimidin-2-yl]-amine.

Part c To (3-chloro-phenyl)-[4-(2-chloro-pyridin-3-yl)-pyrimidin-2-yl]-amine (250 mg, 0.79 mmol) in propane-1,3-diamine (1 mL) is heated in a sealed tube at 110° C. for 30 mins. On cooling the reaction is diluted with methylene chloride and washed with saturated NaHCO₃ to remove excess diamine. The organic layer is concentrated in vacuo and the resulting crude material is purified by flash column chromatography eluting with methanol/methylene chloride to afford N-{3-[2-(3-chloro-phenylamino)-pyrimidin-4-yl]-pyridin-2-yl}-propane-1,3-diamine.

Part d To N-{3-[2-(3-chloro-phenylamino)-pyrimidin-4-yl]-pyridin-2-yl}-propane-1,3-diamine (169 mg, 0.2 mmol) in THF (2 mL) is added acetic anhydride (74 μL, 0.3 mmol) and the resulting solution is stirred at ambient temperature for 20 mins. The solvent is removed in vacuo and the resulting crude material is purified by subjecting to preparative reverse-phase HPLC to afford the title compound. ¹H NMR 400 MHz (CDCl₃) δ 1.70-1.79 (m, 2H), 2.14 (s, 3H), 3.44-3.51 (m, 2H), 3.59-3.66 (m, 2H), 6.57 (dd, J=7.8, 4.8 Hz, 1H), 6.96 (dd, J=7.6, 1.5 Hz, 1H), 7.11 (d, J=5.6 Hz, 1H), 7.23 (t, J=8.1 Hz, 1H), 7.70 (dd, J=8.3, 1.3 Hz, 1H), 7.92 (t, J=2.0 Hz, 1H), 7.97 (dd, J=7.6, 1.5 Hz, 1H), 8.20 (dd, J=4.6, 1.5 Hz, 1H), 8.44 (d, J=5.6 Hz, 1H); MS m/z 397.2 (M+1)

### Example 3

### (3-Chloro-phenyl)-[4-(3-methoxy-pyridin-4-yl)-pyrimidin-2-yl]-amine (45)

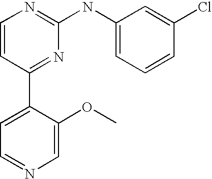

Part a A mixture of (3-methoxy-4-pyridinyl)boronic acid (149 mg, 1.0 mmol), 2,4-dichloropyrimidine (153 mg, 1.0 mmol), sodium carbonate (318 mg, 3.0 mmol) and (Ph₃P)₂PdCl₂ (70 mg, 0.1 μmol) in 1,4-dioxane (4.0 mL) and water (1.0 mL) is degassed (nitrogen) and heated at 95° C. for 8 h. On cooling the solvent is removed in vacuo and the resulting crude solid is purified by subjecting to preparative reverse-phase HPLC to afford 2-chloro-4-(3-methoxy-pyridin-4-yl)pyrimidine.

Part b To 2-chloro-4-(3-methoxy-pyridin-4-yl)pyrimidine (18 mg, 0.08 mmol) and para-toluenesulfonic acid monohydrate (19 mg, 0.101 mmol) in acetonitrile (1 mL) is added 3-chloroaniline (17 mg, 0.13 mmol). The resultant reaction mixture is heated in a sealed tube at 110° C. for 3 h. On cooling the solvent is removed in vacuo and the resulting crude solid is purified by subjecting to preparative reverse-phase HPLC to yield the title compound: ¹H NMR 400 MHz (CDCl₃) δ 4.07 (s, 3H), 7.04 (d, J=8.6 Hz, 1H), 7.26 (t, J=8.1 Hz, 1H) 7.34-7.41 (d, J=8.1 Hz, 1H), 7.49 (d, J=5.1 Hz, 1H), 7.91 (t, J=2.0 Hz, 1H), 8.05 (d, J=5.1 Hz, 1H), 8.44 (d, J=5.1 Hz, 1H), 8.49 (s, 1H), 8.54 (d, J=5.1 Hz, 1H); MS m/z 313.1 (M+1).

The procedures described in the above examples, using appropriate starting materials, can be used to prepare the compounds of Formulas I, II and III. The spectroscopic data shown in Table E are for a selection of the compounds of the invention.

TABLE E

| Compound | | |
|---|---|---|
| number | ¹H NMR 400 MHz | MS (m/z) |
| 43 | | 318.2 (M + 1) |
| 44 | (CDCl₃) δ 2.45 (s, 3H), 6.90 (d, J = 5.1 Hz, 1H), | 297.2 (M + 1) |
| | 7.01 (d, J = 8.1 Hz, 1H), 7.22 (d, J = 8.1 Hz, 1H), | |
| | 7.35 (d, J = 5.1 Hz, 1H), 7.41 (dd, J = 7.8, 1.8 Hz, | |
| | 1H), 7.86 (t, J = 2.0 Hz, 1H), 8.55 (t, J = 5.1 Hz, 2H) | |
| 45 | (CDCl₃) δ 4.07 (s, 3H), 7.04 (d, J = 8.6 Hz, 1H), | 313.1 (M + 1) |
| | 7.26 (t, J = 8.1 Hz, 1H) 7.34-7.41 (d, J = 8.1 Hz, | |
| | 1H), 7.49 (d, J = 5.1 Hz, 1H), 7.91 (t, J = 2.0 Hz, | |
| | 1H), 8.05 (d, J = 5.1 Hz, 1H), 8.44 (d, J = 5.1 Hz, | |
| | 1H), 8.49 (s, 1H), 8.54 (d, J = 5.1 Hz, 1H) | |
| 46 | (CDCl₃) δ 3.07 (s, 3H), 4.08 (s, 3H), 7.50-7.55 (m, | 357.1 (M + 1) |
| | 2H), 7.58-7.63 (m, 2H), 7.72 (d, J = 8.1 Hz, 1H), | |
| | 8.22 (d, J = 5.1 Hz, 1H), 8.46-8.52 (m, 2H), 8.58 (d, | |
| | J = 5.1 Hz, 1H), 8.68 (s, 1H) | |
| 47 | (MeOD) δ 2.01 (s, 3H), 3.92 (s, 3H), 7.34-7.40 (m, | 372.1 (M + 1) |
| | 2H), 7.43 (d, J = 7.6 Hz, 1H), 7.82 (d, J = 8.1 Hz, | |
| | 1H), 8.30 (d, J = 5.1 Hz, 1H), 8.36 (s, 2H), 8.42 (s, | |
| | 1H) | |
| 48 | (MeOD) δ 6.95 (d, J = 8.1 Hz, 1H), 7.17-7.25 (m, | 333.3 (M + 1) |
| | 2H), 7.58 (d, J = 8.6 Hz, 1H), 7.67 (t, J = 7.6 Hz, | |
| | 1H), 7.72 (d, J = 4.1 Hz, 1H), 7.84 (t, J = 7.6 Hz, | |
| | 1H), 7.99 (s, 1H), 8.14 (d, J = 8.6 Hz, 1H), 8.39 (d, J = 8.6 Hz, | |
| | 1H), 8.67 (d, J = 4.6 Hz, 1H), 8.98 (d, J = 4.04 Hz, | |
| | 1H) | |
| 49 | ¹H NMR (400 MHz, MeOD) δ 3.09 (s, 6H), | 326.3 (M + 1) |
| | 7.02 (dd, J = 7.6, 2.0 Hz, 1H), 7.15 (d, J = 5.1 Hz, 1H), | |
| | 7.23 (m, 1H), 7.28 (t, J = 8.1 Hz, 1H), 7.53 (dd, J = 8.3, | |
| | 2.3 Hz, 1H), 8.02 (m, 1H), 8.17 (d, J = 6.1 Hz, | |
| | 1H), 8.28 (d, J = 7.6 Hz, 1H), 8.62 (d, J = 5.1 Hz, 1H) | |
| 50 | (CDCl₃) δ 0.79-0.91 (m, 2H), 1.05-1.15 (m, 3H), | 394.2 (M + 1) |
| | 1.33 (m, 1H), 1.59-1.70 (m, 5H), 3.34 (dd, J = 6.6, | |
| | 5.6 Hz, 2H), 6.57 (dd, J = 7.6, 5.1 Hz, 1H), | |
| | 7.08 (ddd, J = 8.1, 2.0, 1.0 Hz, 1H), 7.12 (d, J = 5.6 Hz, | |
| | 1H), 7.28 (t, J = 7.8 Hz, 1H) 7.35 (ddd, J = 8.1, 2.0, | |
| | 1.0 Hz, 1H), 7.68 (t, J = 2.0 Hz, 1H), 7.88 (dd, J = 7.8, | |
| | 1.8 Hz, 1H), 8.23 (dd, J = 4.8, 1.8 Hz, 1H), | |
| | 8.44 (d, J = 5.6 Hz, 1H), 8.85 (s, 1H) | |
| 51 | | 496.3 (M + 1) |
| 52 | (CDCl₃) δ 2.96 (t, J = 5.8 Hz, 2H), 3.61 (q, J = 5.6 Hz, | 341.1 (M + 1) |
| | 2H), 6.59 (dd, J = 7.6, 5.1 Hz, 1H), 7.03 (d, J = 7.6 Hz, | |
| | 1H), 7.12 (d, J = 5.6 Hz, 1H), 7.25 (t, J = 8.1 Hz, | |
| | 1H), 7.42 (dd, J = 8.1, 1.5 Hz, 1H), 7.75 (t, J = 2.0 Hz, | |
| | 1H), 7.92 (dd, J = 7.8, 2.0 Hz, 1H), 8.21 (dd, | |
| | J = 4.8, 1.77 Hz, 1H), 8.44 (d, J = 5.6 Hz, 1H); | |
| 53 | (MeOD) δ 2.46 (s, 3H), 2.71 (s, 3H), 2.96 (t, J = 6.2 Hz, | 369.2 (M + 1) |
| | 2H), 3.58 (t, J = 6.2 Hz, 2H), 6.89-6.94 (m, 2H), | |
| | 7.04 (d, J = 5.1 Hz, 1H), 7.20 (t, J = 8.3 Hz, 1H), | |
| | 7.50 (dd, J = 8.1, 1.5 Hz, 1H), 7.86 (dd, J = 7.6, 2.0 Hz, | |
| | 1H), 7.98 (t, J = 2.0 Hz, 1H), 8.20 (dd, J = 4.8, | |
| | 1.8 Hz, 1H), 8.44 (d, J = 5.6 Hz, 1H); | |
| 54 | CDCl₃) δ 2.09 (s, 3H), 3.78 (brs, 2H), 3.86 (brs, 2H), | 383.1 (M + 1) |
| | 6.78 (dd, J = 7.6, 6.1 Hz, 1H), 7.00 (d, J = 7.6 Hz, | |
| | 1H), 7.10 (d, J = 5.6 Hz, 1H), 7.25 (m, 1H), | |
| | 7.49 (m, 1H), 7.68 (d, J = 8.1 Hz, 1H), 8.01 (s, 1H), | |
| | 8.16-8.25 (m, 2H), 8.52 (d, J = 5.1 Hz, 1H) | |
| 55 | (CDCl₃) δ 1.74 (quintet, J = 6.3 Hz, 1H), 2.83 (t, J = 6.1 Hz, | 355.1 (M + 1) |
| | 2H), 3.59 (q, J = 5.7 Hz, 2H), 6.59 (dd, J = 7.6, | |
| | 4.6 Hz, 1H), 7.04 (d, J = 8.1 Hz, 1H), 7.11 (d, J = 5.6 Hz, | |
| | 1H), 7.25 (t, J = 8.1 Hz, 1H), 7.43 (d, J = 8.1 Hz, | |
| | 1H), 7.74 (br.s, 1H), 7.91 (dd, J = 7.8, 1.8 Hz, | |
| | 1H), 8.21 (dd, J = 4.6, 1.5 Hz, 1H), 8.43 (d, J = 5.6 Hz, | |
| | 1H) | |
| 56 | | 369.2 (M + 1) |
| 57 | | 368.4 (M + 1) |
| 58 | (CDCl₃) δ 1.86 (quintet, J = 12.1, 6.1 Hz, 2H), | 399.3 (M + 1) |
| | 2.94 (t, J = 5.8 Hz, 2H), 3.08 (s, 3H), 3.60-3.68 (m, 2H), | |
| | 6.60 (dd, J = 7.3, 5.3 Hz, 1H), 7.17 (d, J = 5.6 Hz, | |
| | 1H), 7.51 (t, J = 8.0 Hz, 1H), 7.57 (d, J = 7.6 Hz, | |
| | 1H), 7.94 (d, J = 7.6 Hz, 1H), 8.03 (d, J = 8.1 Hz, | |
| | 1H), 8.23 (d, J = 5.1 Hz, 1H), 8.30 (s, 1H), 8.46 (d, J = 5.6 Hz, | |
| | 1H) | |
| 59 | (MeOD) δ 1.65 (quintet, J = 6.8 Hz, 2H), 2.63 (t, J = 6.8 Hz, | 400.2 (M + 1) |
| | 2H), 3.51 (t, J = 6.8 Hz, 2H), 6.60 (dd, J = 7.8, | |
| | 4.8 Hz, 1H), 7.24 (d, J = 5.6 Hz, 1H), 7.47 (t, J = 7.8 Hz, | |
| | 1H), 7.55 (d, J = 7.6 Hz, 1H), 7.82 (d, J = 8.6 Hz, | |
| | 1H), 8.03-8.10 (m, 2H), 8.23 (brs, 1H), | |
| | 8.40 (d, J = 5.1 Hz, 1H) | |
| 60 | (MeOD) δ 1.74 (quintet, J = 6.9 Hz, 2H), 2.68 (t, J = 6.8 Hz, | 346.2 (M + 1) |
| | 2H), 3.57 (t, J = 6.8 Hz, 2H), 6.62 (dd, J = 7.8, | |
| | 4.8 Hz, 1H), 7.26-7.33 (m, 2H), 7.44 (t, J = 8.1 Hz, | |
| | 1H), 7.84 (d, J = 8.6 Hz, 1H), 8.05-8.11 (d, J = 7.6 Hz, | |
| | 2H), 8.18 (s, 1H), 8.44 (d, J = 5.6 Hz, 1H) | |
| 61 | (CDCl₃) δ 0.91 (s, 6H), 2.61 (s, 2H), 3.40 (d, J = 6.1 Hz, | 383.2 (M + 1) |
| | 2H), 6.62 (dd, J = 7.6, 5.1 Hz, 1H), 7.07 (m, | |
| | 1H), 7.14 (d, J = 5.6 Hz, 1H), 7.28 (d, J = 8.1 Hz, | |
| | 1H), 7.38 (d, J = 8.1 Hz, 1H), 7.70 (t, J = 2.0 Hz, | |
| | 1H), 7.94 (dd, J = 7.8, 1.8 Hz, 1H), 8.20 (dd, J = 4.6, | |
| | 1.5 Hz, 1H), 8.46 (d, J = 5.6 Hz, 1H) | |
| 62 | (MeOD) δ 2.00 (quintet, J = 6.7 Hz, 2H), 2.68 (s, | 369.2 (M + 1) |
| | 3H), 2.91 (t, J = 6.8 Hz, 2H), 3.61 (t, J = 6.6 Hz, | |
| | 2H), 6.91-6.98 (m, 2H), 7.03 (d, J = 5.1 Hz, 1H), | |
| | 7.23 (t, J = 8.1 Hz, 1H), 7.53 (dd, J = 8.1, 2.0 Hz, | |
| | 1H), 7.89 (dd, J = 7.6, 1.52 Hz, 1H), 8.01 (t, J = 2.0 Hz, | |
| | 1H), 8.22 (dd, J = 5.1, 1.5 Hz, 1H), 8.47 (d, J = 5.1 Hz, | |
| | 1H) | |
| 63 | (MeOD) δ 1.81 (quintet, J = 7.0, 2H), 2.42 (s, 6H), | 383.2 (M + 1) |
| | 2.58 (t, J = 7.0, 2H), 3.55 (t, J = 6.8 Hz, 2H), | |
| | 6.68 (dd, J = 6.1, 6.1 Hz, 1H), 7.04 (dd, J = 8.1, 1.5 Hz, | |
| | 1H), 7.28-7.34 (m, 2H), 7.49 (d, J = 8.1, 1.0 Hz, | |
| | 1H), 7.83 (brs, 1H), 8.14 (d, J = 6.6 Hz, 2H), | |
| | 8.46 (d, J = 5.6 Hz, 1H) | |
| 64 | (MeOD) δ 1.25 (d, J = 6.6 Hz, 6H), 1.85-1.94 (m, | 397.2 (M + 1) |
| | 2H), 2.93 (t, J = 7.1, 2H), 3.25 (overlapping w/ | |
| | solvent, 1H), 3.61 (t, J = 6.3 Hz, 2H), 6.71 (dd, J = 7.6, | |
| | 5.1 Hz, 1H), 7.02 (dd, J = 7.6, 1.5 Hz, 1H), | |
| | 7.25-7.32 (m, 2H), 7.46 (dd, J = 8.1, 2.0 Hz, 1H), | |
| | 7.80 (t, J = 2.0 Hz, 1H), 8.8.11-8.18 (m, 2H), | |
| | 8.45 (d, J = 5.6 Hz, 1H) | |
| 65 | (CDCl₃) δ 1.70-1.79 (m, 2H), 2.14 (s, 3H), | 397.2 (M + 1) |
| | 3.44-3.51 (m, 2H), 3.59-3.66 (m, 2H), 6.57 (dd, J = 7.8, 4.8 Hz, | |
| | 1H), 6.96 (dd, J = 7.6, 1.5 Hz, 1H), 7.11 (d, J = 5.6 Hz, | |
| | 1H), 7.23 (t, J = 8.1 Hz, 1H), 7.70 (dd, J = 8.3, | |
| | 1.3 Hz, 1H), 7.92 (t, J = 2.0 Hz, 1H), 7.97 (dd, J = 7.6, | |
| | 1.5 Hz, 1H), 8.20 (dd, J = 4.6, 1.5 Hz, 1H), | |
| | 8.44 (d, J = 5.6 Hz, 1H) | |
| 66 | (MeOD) δ 1.67 (quintet, J = 6.6 Hz, 2H), 1.86 (s, | 411.2 (M + 1) |
| | 3H), 2.11 (s, 3H), 3.16 (t, J = 6.6 Hz, 2H), 3.37 (t, J = 6.6 Hz, | |
| | 2H), 6.64 (dd, J = 7.1, 5.1 Hz, 1H), | |
| | 6.89 (d, J = 8.1 Hz, 1H), 7.18 (t, J = 8.1 Hz, 1H), | |
| | 7.45-7.50 (m, 2H), 7.88 (t, J = 2.0 Hz, 1H), 8.04 (dd, J = 5.1, | |
| | 1.5 Hz, 1H), 8.38 (s, 1H) | |
| 67 | (CDCl₃) δ 1.74-1.82 (m, 2H), 2.22 (s, 3H), 3.06 (s, | 441.4 (M + 1) |
| | 3H), 3.46-3.54 (m, 2H), 3.63-3.69 (m, 2H), 6.59 (dd, | |
| | J = 7.6, 4.6 Hz, 1H), 7.16 (d, J = 5.6 Hz, 1H), | |
| | 7.50 (d, J = 5.1 Hz, 2H), 8.01 (d, J = 7.6 Hz, 1H), | |
| | 8.21 (dd, J = 4.6, 1.5 Hz, 1H), 8.33-8.39 (m, 2H), 8.47 (d, | |
| | J = 5.6 Hz, 1H) | |
| 68 | (MeOD) δ 1.66 (quintet, J = 6.6 Hz, 2H), 2.03 (s, | 442.1 (M + 1) |
| | 3H), 3.29 (t, J = 7.1 Hz, 2H), 3.51 (t, J = 6.1 Hz, | |
| | 2H), 6.62 (dd, J = 7.8, 4.8 Hz, 1H), 7.31 (d, J = 5.6 Hz, | |
| | 1H), 7.45 (t, J = 8.0 Hz, 1H), 7.50 (d, J = 7.6 Hz, | |
| | 1H), 8.02 (d, J = 7.6 Hz, 1H), 8.09 (dd, J = 5.1, 1.5 Hz, | |
| | 1H), 8.14 (dd, J = 8.1, 1.5 Hz, 1H), 8.28 (s, 1H), | |
| | 8.45 (d, J = 5.6 Hz, 1H) | |
| 69 | (MeOD) δ 1.72 (quintet, J = 6.3 Hz, 2H), 2.02 (s, | 388.2 (M + 1) |
| | 3H), 3.31 (t, J = 6.1 Hz, 2H), 3.54 (t, J = 6.1 Hz, | |
| | 2H), 6.77 (dd, J = 7.6, 5.6 Hz, 1H), 7.30 (d, J = 7.6 Hz, | |
| | 1H), 7.38 (d, J = 5.6 Hz, 1H), 7.44 (t, J = 8.1 Hz, | |
| | 1H), 7.91 (dd, J = 8.6, 1.5 Hz, 1H), 8.05 (dd, J = 5.6, | |
| | 1.5 Hz, 1H), 8.28 (t, J = 1.8 Hz, 1H), 8.34 (d, J = 7.1 Hz, | |
| | 1H), 8.53 (d, J = 5.6 Hz, 1H) | |
| 70 | (CDCl₃) δ 1.29 (s, 9H), 1.80 (brs, 2H), 3.50 (q, J = 6.1 Hz, | 439.2 (M + 1) |
| | 2H), 3.68 (brs, 2H), 6.07 (brs, 1H), 6.65 (brs, | |
| | 1H), 7.00 (d, J = 8.6 Hz, 1H), 7.13 (d, J = 5.6 Hz, | |
| | 1H), 7.23 (d, J = 8.1 Hz, 1H), 7.73 (d, J = 8.1 Hz, | |
| | 1H), 7.83 (s, 1H), 8.22 (d, J = 5.1 Hz, 1H), 8.47 (d, J = 5.6 Hz, | |
| | 1H) | |
| 71 | (CDCl₃) δ 1.09 (s, 9H), 1.81 (brs, 2H), 2.22 (s, 2H), | 453.2 (M + 1) |
| | 3.48 (q, J = 6.6 Hz, 2H), 3.72 (brs, 2H), 6.65 (t, J = 6.6 Hz, | |
| | 1H), 6.99 (d, J = 8.1 Hz, 1H), 7.12 (d, J = 5.6 Hz, | |
| | 1H), 7.22 (d, J = 8.1 Hz, 1H), 7.76 (d, J = 8.1 Hz, | |
| | 1H), 7.85 (t, J = 2.0 Hz, 1H), 8.07 (brs, 1H), | |
| | 8.22 (dd, J = 5.1, 1.5 Hz, 1H), 8.47 (d, J = 5.6 Hz, | |
| | 1H) | |
| 72 | (CDCl₃) δ 1.83 (quintet, J = 5.7 Hz, 2H), 2.92 (s, | 433.1 (M + 1) |
| | 3H), 3.22 (brd, J = 5.1 Hz, 2H), 3.74 (brd, J = 5.1 Hz, | |
| | 2H), 6.65 (dd, J = 7.6, 5.1 Hz, 1H), 7.06 (d, J = 8.1 Hz, | |
| | 1H), 7.12 (d, J = 5.6 Hz, 1H), 7.28 (d, J = 8.1 Hz, | |
| | 1H), 7.43 (d, J = 8.1 Hz, 1H), 7.83 (s, 1H), | |
| | 7.98 (d, J = 6.6 Hz, 1H), 8.22 (dd, J = 5.1, 1.5 Hz, | |
| | 1H), 8.47 (d, J = 5.6 Hz, 1H) | |
| 73 | (CDCl₃) δ 0.84 (s, 6H), 2.13 (s, 3H), 3.08 (d, J = 7.1 Hz, | 425.2 (M + 1) |
| | 2H), 3.47 (d, J = 6.6 Hz, 2H), 6.56 (dd, J = 7.6, | |
| | 4.6 Hz, 1H), 6.98 (dd, J = 8.3, 1.8 Hz, 1H), 7.12 (d, | |
| | J = 5.6 Hz, 1H), 7.22 (t, J = 8.1 Hz, 1H), 7.55 (dd, J = 8.1, | |
| | 1.0 Hz, 1H), 7.81 (t, J = 2.0 Hz, 1H), 7.96 (dd, | |
| | J = 7.8, 1.8 Hz, 1H) 8.16 (dd, J = 4.8, 1.8 Hz, 1H), | |
| | 8.41 (d, J = 5.6 Hz, 1H) | |
| 74 | (CDCl₃) δ 1.72 (quintet, J = 5.7 Hz, 2H), | 356.1 (M + 1). |
| | 3.57-3.68 (m, 4H), 6.60 (dd, J = 7.8, 4.8 Hz, 1H), 7.08 (d, J = 6.6 Hz, | |
| | 1H), 7.12 (d, J = 5.6 Hz, 1H), 7.28 (t, J = 8.1 Hz, | |
| | 1H), 7.40 (dd, J = 8.1, 1.5 Hz, 1H), 7.68 (t, J = 2.0 Hz, | |
| | 1H), 7.92 (dd, J = 7.6, 2.0 Hz, 1H), 8.16 (dd, | |
| | J = 4.8, 1.8 Hz, 1H), 8.44 (d, J = 5.6 Hz, 1H) | |
| 75 | (CDCl₃) δ 1.94 (quintet, J = 6.0 Hz, 2H), 3.35 (s, 3H), | 370.1 (M + 1) |
| | 3.63 (t, J = 5.6 Hz, 2H), 3.66-3.74 (m, 2H), 6.62 (dd, | |
| | J = 7.3, 5.3 Hz, 1H), 7.04 (d, J = 7.6 Hz, 1H), | |
| | 7.15 (d, J = 5.6 Hz, 1H), 7.27 (t, J = 8.1 Hz, 1H), 7.43 (d, | |
| | J = 8.1 Hz, 1H), 7.81 (t, J = 1.8 Hz, 1H), 7.98 (d, J = 7.6 Hz, | |
| | 1H), 8.24 (dd, J = 4.8, 1.8 Hz, 1H), 8.47 (d, | |
| | J = 5.6 Hz, 1H) | |
| 76 | (MeOD) δ 1.83 (quintet, J = 6.5 Hz, 2H), 2.00 (s, | 398.1 (M + 1) |
| | 3H), 3.55 (t, J = 6.8 Hz, 2H), 4.12 (t, J = 6.1 Hz, | |
| | 2H), 6.66 (m, 1H), 7.03 (d, J = 8.1 Hz, 1H), 7.30 (m, | |
| | 2H), 7.50 (d, J = 8.6 Hz, 1H), 7.84 (brs, 1H), | |
| | 8.12 (d, J = 6.6 Hz, 2H), 8.46 (d, J = 5.6 Hz, 1H) | |
| 77 | (CDCl₃) δ 1.83 (brs, 2H), 2.07 (quintet, J = 7.83 Hz, | 423.2 (M + 1) |
| | 2H), 2.51 (t, J = 8.1 Hz, 2H), 3.37-3.47 (m, 4H), | |
| | 3.61 (brs, 2H), 6.59 (brs, 1H), 6.90 (d, J = 7.6 Hz, | |
| | 1H), 7.05 (d, J = 5.6 Hz, 1H), 7.16 (t, J = 8.1 Hz, | |
| | 1H), 7.65 (d, J = 8.1 Hz, 1H), 7.87 (t, J = 1.78 Hz, | |
| | 1H), 8.02 (brs, 1H), 8.17 (d, J = 5.1 Hz, 1H), | |
| | 8.42 (d, J = 5.6 Hz, 1H) | |
| 78 | (CDCl₃) δ 1.74 (brs, 2H), 2.43 (brs, 6H), 3.59 (q, J = 6.6 Hz, | 425.2 (M + 1) |
| | 2H), 3.70 (brs, 4H), 6.59 (dd, J = 7.6, 4.6 Hz, | |
| | 1H), 7.05 (d, J = 8.1 Hz, 1H), 7.12 (d, J = 5.6 Hz, | |
| | 1H), 7.28 (d, J = 8.1 Hz, 1H), 7.44 (d, J = 8.1 Hz, | |
| | 1H), 7.76 (s, 1H), 7.89 (dd, J = 7.6, 1.5 Hz, 1H), | |
| | 8.22 (dd, J = 4.6, 1.5 Hz, 1H), 8.43 (d, J = 5.1 Hz, | |
| | 1H) | |
| 79 | (MeOD) δ 2.79 (t, J = 7.1 Hz, 2H), 3.69 (t, J = 7.1 Hz, | 392.1 (M + 1) |
| | 2H), 6.63 (dd, J = 7.6, 5.1 Hz, 1H), 6.68 (s, 1H), | |
| | 6.99 (dd, J = 8.1, 2.02 Hz, 1H), 7.22-7.29 (m, 2H), | |
| | 7.43-7.49 (m, 2H), 7.83 (s, 1H), 8.06-8.12 (m, 2H), | |
| | 8.41 (d, J = 5.6 Hz, 1H) | |
| 80 | (CDCl₃) δ 1.96 (quintet, J = 6.8 Hz, 2H), 3.46 (q, J = 6.6 Hz, | 406.2 (M + 1) |
| | 2H), 3.90 (t, J = 6.8 Hz, 2H), 6.62 (dd, J = 7.6, | |
| | 4.6 Hz, 1H), 6.84 (s, 1H), 7.04 (s, 1H), 7.05 (d, | |
| | J = 7.6 Hz, 1H), 7.11 (d, J = 5.6 Hz, 1H), 7.24 (t, J = 7.8 Hz, | |
| | 1H), 7.40 (dd, J = 8.1, 1.5 Hz, 1H), 7.47 (s, | |
| | 1H), 7.77 (t, J = 2.0 Hz, 1H), 7.89 (dd, J = 7.8, 1.8 Hz, | |
| | 1H), 8.21 (dd, J = 4.8, 1.8 Hz, 1H), 8.44 (d, J = 5.6 Hz, | |
| | 1H) | |
| 81 | MeOD) δ 1.89 (quintet, J = 7.1 Hz, 2H), 2.17 (s, | 420.2 (M + 1) |
| | 3H), 3.46 (t, J = 6.8 Hz, 2H), 3.84 (t, J = 7.1 Hz, | |
| | 2H), 6.64 (dd, J = 7.6, 5.1 Hz, 1H), 6.68 (d, J = 1.0 Hz, | |
| | 1H), 6.84 (d, J = 1.5 Hz, 1H), 6.98 (dd, J = 7.6, | |
| | 1.52 Hz, 1H), 7.22 (d, J = 8.1 Hz, 1H), 7.27 (d, J = 5.0 Hz, | |
| | 1H), 7.42 (dd, J = 8.1, 2.0 Hz, 1H), 7.75 (t, J = 1.8 Hz, | |
| | 1H), 8.06-8.11 (m, 2H), 8.43 (d, J = 5.6 Hz, | |
| | 1H) | |
| 82 | (MeOD) δ 1.92 (quintet, J = 7.0 Hz, 2H), 2.12 (s, | 434.2 (M + 1) |
| | 3H), 2.17 (s, 3H), 3.36 (t, J = 6.8 Hz, 2H), 3.86 (t, J = 7.1 Hz, | |
| | 2H), 6.65-6.70 (m, 2H), 6.85 (br.s, 1H), | |
| | 6.88 (d, J = 8.1 Hz, 1H), 7.16 (t, J = 8.1 Hz, 1H), | |
| | 7.44 (dd, J = 8.1, 2.0 Hz, 1H), 7.49 (dd, J = 7.6, 1.5 Hz, | |
| | 1H), 7.88 (t, J = 2.0 Hz, 1H), 8.06 (dd, J = 5.1, | |
| | 2.0 Hz, 1H), 8.40 (s, 1H) | |
| 83 | (MeOD) δ 1.93 (quintet, J = 7.0 Hz, 2H), 3.45 (t, J = 7.0 Hz, | 451.2 (M + 1) |
| | 2H), 3.98 (t, J = 7.1 Hz, 2H), 6.64 (dd, J = 7.6, | |
| | 4.6 Hz, 1H), 6.86 (s, 1H), 7.01 (s, 1H), 7.28 (d, | |
| | J = 5.6 Hz, 1H), 7.44 (t, J = 7.8 Hz, 1H), | |
| | 7.50-7.56 (m, 2H), 7.78 (d, J = 7.6 Hz, 1H), 8.05-8.12 (m, 2H), | |
| | 8.24 (brs, 1H), 8.44 (d, J = 5.6 Hz, 1H) | |
| 84 | (CDCl₃) δ 1.45-1.60 (m, 4H), 2.71 (t, J = 6.3 Hz, | 369.3 (M + 1) |
| | 2H), 3.50 (q, J = 6.0 Hz, 2H), 6.58 (dd, J = 7.6, 5.1 Hz, | |
| | 1H), 7.06 (d, J = 7.1 Hz, 1H), 7.10 (d, J = 5.6 Hz, | |
| | 1H), 7.26 (t, J = 8.1 Hz, 1H), 7.40 (dd, J = 8.1, | |
| | 2.0 Hz, 1H), 7.70 (t, J = 2.0 Hz, 1H), 7.87 (dd, J = 7.6, | |
| | 1.5 Hz, 1H), 8.22 (dd, J = 4.8, 1.8 Hz, 1H), | |
| | 8.42 (d, J = 5.6 Hz, 1H) | |
| 85 | (MeOD) δ 1.46 (brs, 4H), 1.86 (s, 3H), 3.09 (t, J = 6.6 Hz, | 411.2 (M + 1) |
| | 2H), 3.45 (t, J = 6.8 Hz, 2H), 6.74 (dd, J = 7.6, | |
| | 5.6 Hz, 1H), 7.03 (dd, J = 8.1, 2.0 Hz, 1H), | |
| | 7.25-7.32 (m, 2H), 7.44 (dd, J = 8.1, 2.0 Hz, 1H), | |
| | 7.77 (t, J = 1.8 Hz, 1H), 8.04 (dd, J = 5.3, 1.3 Hz, | |
| | 1H), 8.26 (d, J = 7.1 Hz, 1H), 8.47 (d, J = 5.6 Hz, | |
| | 1H); | |
| 86 | (MeOD) δ 3.98 (s, 3H) 7.05 (ddd, J = 7.5, 5.1, 2.5 Hz, | 203.3 (M + 1) |
| | 1H), 7.26 (dd, J = 5.1, 2.5 Hz, 1H), | |
| | 8.18-8.24 (m, 2H), 8.29 (dd, J = 7.6, 2.0 Hz, 1H) | |
| 87 | (MeOD) δ 2.93 (s, 3H), 3.99 (s, 3H), 7.05 (ddd, J = 7.5, | 217.3 (M + 1) |
| | 4.9, 2.3 Hz, 1H), 7.25 (d, J = 5.1 Hz, 1H), | |
| | 8.17-8.24 (m, 2H), 8.38 (d, J = 7.1 Hz, 1H) | |
| 88 | (MeOD) δ 1.20-1.46 (m, 6H), 1.63 (d, J = 13.6 Hz, | 285.2 (M + 1) |
| | 1H), 1.76 (d, J = 13.6 Hz, 2H), 2.00 (d, J = 11.6 Hz, | |
| | 2H), 3.98 (s, 3H), 7.05 (ddd, J = 7.3, 5.1, 2.3 Hz, | |
| | 1H), 7.21 (d, J = 5.1 Hz, 1H), 8.16-8.22 (m, 2H), | |
| | 8.33 (d, J = 7.6 Hz, 1H) | |
| 89 | | 280.1 (M + 1) |
| 90 | (MeOD) δ 4.10 (s, 3H), 7.22 (dd, J = 8.1, 5.1 Hz, | 280.3 (M + 1) |
| | 1H), 7.86-7.90 (m, 2H), 8.38 (dd, J = 5.1, 2.0 Hz, | |
| | 1H), 8.53 (d, J = 5.6 Hz, 1H), 8.76 (dd, J = 7.6, 2.0 Hz, | |
| | 1H), 9.05 (d, J = 5.1 Hz, 1H), 9.33 (m, 1H), | |
| | 9.48 (br.s, 1H) | |
| 91 | (MeOD) δ 4.03 (s, 3H), 7.10 (dd, J = 7.8, 4.8 Hz, | 304.2 (M + 1) |
| | 1H), 7.27 (d, J = 8.1 Hz, 1H), 7.42 (t, J = 8.0 Hz, | |
| | 1H), 7.54 (d, J = 5.6 Hz, 1H), 7.90 (d, J = 8.0 Hz, | |
| | 1H), 8.24 (d, J = 5.1 Hz, 1H), 8.31 (s, 1H), 8.43 (d, J = 5.6 Hz, | |
| | 1H), 8.48 (d, J = 5.1 Hz, 1H) | |
| 92 | (MeOD) δ 4.04 (s, 3H), 7.13 (dd, J = 7.6, 5.1 Hz, | 324.1 (M + 1) |
| | 1H), 7.48 (t, J = 8.1 Hz, 1H), 7.62 (d, J = 5.1 Hz, | |
| | 1H), 7.80 (dd, J = 8.1, 2.0 Hz, 1H), 7.89 (dd, J = 8.1, | |
| | 2.0 Hz, 1H), 8.25 (dd, J = 4.8, 1.8 Hz, 1H), 8.50 (d, | |
| | J = 5.6 Hz, 1H), 8.59 (dd, J = 7.6, 2.0 Hz, 1H), | |
| | 9.08 (t, J = 2.3 Hz, 1H) | |
| 93 | (MeOD) δ 3.09 (s, 3H), 4.03 (s, 3H), 7.12 (dd, J = 7.6, | 357.2 (M + 1). |
| | 5.1 Hz, 1H), 7.49-7.54 (m, 2H), 7.62 (d, J = 5.1 Hz, | |
| | 1H), 7.81 (m, 1H), 8.23 (d, J = 5.0 Hz, 1H), | |
| | 8.48 (d, J = 5.0 Hz, 1H), 8.61 (d, J = 7.6 Hz, 1H), | |
| | 8.78 (br.s, 1H) | |
| 94 | 400 MHz (MeOD) δ 4.06 (s, 3H), 7.13 (dd, J = 7.3, | 323.2 (M + 1) |
| | 4.8 Hz, 1H), 7.36 (t, J = 8.1 Hz, 1H), 7.57 (d, J = 5.1 Hz, | |
| | 1H), 7.64 (d, J = 8.1 Hz, 1H), 7.85 (d, J = 8.1 Hz, | |
| | 1H), 8.25 (dd, J = 4.8, 1.8 Hz, 1H), 8.45 (d, J = 5.1 Hz, | |
| | 1H), 8.50 (brs, 1H), 8.61 (dd, J = 7.6, 2.0 Hz, | |
| | 1H) | |
| 95 | (MeOD) δ 4.03 (s, 3H), 7.11 (ddd, J = 7.2, 4.9, 2.0 Hz, | 322.3 (M + 1) |
| | 1H), 7.36 (t, J = 8.1 Hz, 1H), 7.44 (d, J = 7.6 Hz, | |
| | 1H), 7.54 (d, J = 5.6 Hz, 1H), 7.84 (d, J = 7.6 Hz, | |
| | 1H), 8.23 (m, 1H), 8.32 (s, 1H), 8.44 (dd, J = 5.1, | |
| | 2.0 Hz, 1H), 8.54 (dd, J = 7.6, 2.0 Hz, 1H) | |
| 96 | (MeOD) δ 1.20 (t, J = 7.3 Hz, 3H), 3.38 (q, J = 7.2 Hz, | 350.2 (M + 1) |
| | 2H), 4.03 (s, 3H), 7.11 (dd, J = 7.6, 5.1 Hz, 1H), | |
| | 7.34-7.38 (m, 2H), 7.53 (d, J = 5.1 Hz, 1H), 7.81 (dt, | |
| | J = 7.1, 2.0 Hz, 1H), 8.21-8.26 (m, 2H), 8.43 (d, J = 5.1 Hz, | |
| | 1H), 8.53 (dd, J = 7.6, 1.5 Hz, 1H) | |
| 97 | (MeOD) δ 2.31 (s, 6H), 2.58 (t, J = 6.8 Hz, 2H), | 393.2 (M + 1) |
| | 3.51 (t, J = 6.6 Hz, 2H), 4.03 (s, 3H), 7.11 (dd, J = 7.6, | |
| | 5.1 Hz, 1H), 7.33-7.42 (m, 2H), 7.53 (d, J = 5.6 Hz, | |
| | 1H), 7.84 (dt, J = 7.6, 1.8 Hz, 1H), 8.23 (dd, J = 5.1, | |
| | 2.0 Hz, 1H), 8.26 (brs, 1H), 8.43 (d, J = 5.6 Hz, | |
| | 1H), 8.52 (dd, J = 7.6, 2.0 Hz, 1H) | |
| 98 | | 336.3 (M + 1) |
| 99 | (MeOD) δ 4.03 (s, 3H), 7.12 (dd, J = 7.3, 4.8 Hz, | 358.1 (M + 1) |
| | 1H), 7.42 (t, J = 7.8 Hz, 1H), 7.48 (d, J = 8.1 Hz, | |
| | 1H), 7.60 (d, J = 5.1 Hz, 1H), 7.73 (d, J = 8.1 Hz, | |
| | 1H), 8.23 (dd, J = 5.1, 2.0 Hz, 1H), 8.46 (d, J = 5.6 Hz, | |
| | 1H), 8.63 (dd, J = 7.6, 2.0 Hz, 1H), 8.66 (br.s, | |
| | 1H) | |
| 100 | (MeOD) δ 2.51 (s, 3H), 4.03 (s, 3H), 7.12 (dd, J = 7.6, | 372.2 (M + 1) |
| | 5.1 Hz, 1H), 7.40 (d, J = 8.1 Hz, 1H), 7.46 (t, | |
| | J = 7.8 Hz, 1H), 7.60 (d, J = 5.6 Hz, 1H), 7.74 (d, J = 8.1 Hz, | |
| | 1H), 8.23 (dd, J = 4.8, 1.8 Hz, 1H), 8.47 (d, | |
| | J = 5.6 Hz, 1H), 8.61 (dd, J = 7.6, 1.5 Hz, 1H), | |
| | 8.65 (s, 1H) | |
| 101 | (MeOD) δ 1.02 (t, J = 7.1 Hz, 3H), 2.90 (q, J = 7.6 Hz, | 386.2 (M + 1) |
| | 2H), 4.04 (s, 3H), 7.13 (dd, J = 7.6, 5.1 Hz, 1H), | |
| | 7.39-7.48 (m, 2H), 7.60 (d, J = 5.1 Hz, 1H), 7.73 (d, | |
| | J = 8.1 Hz, 1H), 8.24 (dd, J = 4.8, 1.8 Hz, 1H), | |
| | 8.47 (d, J = 5.1 Hz, 1H), 8.60 (d, J = 7.6, 2.0 Hz, 1H), | |
| | 8.66 (brs, 1H) | |
| 102 | (MeOD) δ 0.78 (t, J = 7.3 Hz, 3H), 1.23 (sextet, J = 7.1 Hz, | 414.2 (M + 1) |
| | 2H), 1.37 (quintet, J = 7.3 Hz, 2H), 2.84 (t, J = 7.1 Hz, | |
| | 2H), 4.03 (s, 3H), 7.12 (dd, J = 7.3, 4.8 Hz, | |
| | 1H), 7.38-7.47 (m, 2H), 7.59 (d, J = 5.6 Hz, | |
| | 1H), 7.71 (brd, J = 7.6 Hz, 1H), 8.23 (dd, J = 5.1, | |
| | 2.0 Hz, 1H), 8.46 (d, J = 5.1 Hz, 1H), 8.61 (dd, J = 7.6, | |
| | 2.0 Hz, 1H), 8.68 (brs, 1H) | |
| 103 | (MeOD) δ 2.96 (t, J = 6.1 Hz, 2H), 3.50 (t, J = 6.1 Hz, | 402.1 (M + 1) |
| | 2H), 4.03 (s, 3H), 7.13 (dd, J = 7.6, 4.6 Hz, 1H), | |
| | 7.40-7.48 (m, 2H), 7.60 (d, J = 5.1 Hz, 1H), 7.74 (dt, | |
| | J = 7.1, 2.0 Hz, 1H), 8.24 (dd, J = 5.1, 2.0 Hz, 1H), | |
| | 8.47 (d, J = 5.6 Hz, 1H), 8.61 (dd, J = 7.6, 2.0 Hz, | |
| | 1H), 8.66 (br.s, 1H) | |
| 104 | (MeOD) δ 1.00-1.1.20 (m, 5H), 1.40-1.70 (m, 5H), | 440.3 (M + 1) |
| | 3.03 (m, 1H), 4.04 (s, 3H), 7.13 (dd, J = 7.6, 5.1 Hz, | |
| | 1H), 7.43 (d, J = 5.1 Hz, 2H), 7.60 (d, J = 5.1 Hz, | |
| | 1H), 7.69 (m, 1H), 8.24 (dd, J = 5.1, 2.0 Hz, 1H), | |
| | 8.47 (d, J = 5.6 Hz, 1H), 8.63 (dd, J = 7.6, 2.0 Hz, | |
| | 1H), 8.74 (s, 1H) | |
| 105 | MHz (MeOD) δ 1.05 (t, J = 7.3 Hz, 6H), 3.19 (q, J = 7.1 Hz, | 414.2 (M + 1) |
| | 4H), 4.03 (s, 3H), 7.12 (dd, J = 7.6, 5.1 Hz, | |
| | 1H), 7.36 (d, J = 7.6 Hz, 1H), 7.44 (t, J = 7.8 Hz, | |
| | 1H), 7.58 (d, J = 5.1 Hz, 1H), 7.68 (d, J = 8.1 Hz, | |
| | 1H), 8.24 (dd, J = 5.1, 2.0 Hz, 1H), 8.47 (d, J = 5.6 Hz, | |
| | 1H), 8.58 (dd, J = 7.6, 1.5 Hz, 1H), 8.74 (s, 1H) | |
| 106 | (MeOD) δ 1.95-2.02 (m, 4H), 3.24-3.30 (overlap w/ | 348.2 (M + 1) |
| | solvent, 4H), 4.02 (s, 3H), 6.24 (dd, J = 7.8, 2.3 Hz, | |
| | 1H), 6.84 (d, J = 8.1 Hz, 1H), 7.04-7.10 (m, 2H), | |
| | 7.19 (brs, 1H), 7.45 (d, J = 5.1 Hz, 1H), 8.23 (dd, J = 4.8, | |
| | 1.8 Hz, 1H), 8.37 (d, J = 5.1 Hz, 1H), 8.52 (dd, | |
| | J = 7.6, 1.5 Hz, 1H) | |
| 107 | (MeOD) δ 1.33-1.42 (m, 2H), 1.55 (quintet, J = 5.7 Hz, | 426.2 (M + 1) |
| | 4H), 2.95 (t, J = 5.6 Hz, 4H), 4.03 (s, 3H), | |
| | 7.11 (dd, J = 7.6, 5.1 Hz, 1H), 7.31 (d, J = 7.6 Hz, 1H), | |
| | 7.47 (t, J = 8.1 Hz, 1H), 7.58 (d, J = 5.1 Hz, 1H), | |
| | 7.72 (dd, J = 8.3, 2.3 Hz, 1H), 8.24 (dd, J = 5.1, 2.0 Hz, | |
| | 1H), 8.47 (d, J = 5.6 Hz, 1H), 8.56 (dd, J = 7.6, | |
| | 2.0 Hz, 1H), 8.66 (brs, 1H) | |
| 108 | (MeOD) δ 1.35 (t, J = 7.3 Hz, 3H), 4.03 (s, 3H), | 351.2 (M + 1) |
| | 4.35 (q, J = 7.2 Hz, 2H), 7.11 (dd, J = 7.6, 4.6 Hz, | |
| | 1H), 7.37 (t, J = 7.6 Hz, 1H), 7.55 (d, J = 5.1 Hz, | |
| | 1H), 7.61 (d, J = 7.6 Hz, 1H), 7.84 (d, J = 8.0 Hz, | |
| | 1H), 8.24 (dd, J = 4.6, 1.5 Hz, 1H), 8.44 (d, J = 5.6 Hz, | |
| | 1H), 8.54-8.59 (m, 2H) | |
| 109 | (MeOD) δ 1.55 (s, 9H), 4.03 (s, 3H), 7.09 (ddd, J = 7.6, | 379.3 (M + 1) |
| | 4.6, 2.0 Hz, 1H), 7.34 (td, J = 8.0, 2.3 Hz, 1H), | |
| | 7.51-7.57 (m, 2H), 7.85 (d, J = 8.6 Hz, 1H), 8.23 (m, | |
| | 1H), 8.40-8.45 (m, 2H), 8.53 (d, J = 7.6 Hz, 1H) | |
| 110 | | 346.3 (M + 1) |
| 111 | (MeOD) δ 3.77 (s, 3H), 4.00 (s, 3H), 7.09 (dd, J = 7.3, | 360.2 (M + 1) |
| | 4.8 Hz, 1H), 7.28 (d, J = 7.1 Hz, 1H), 7.47 (t, J = 7.8 Hz, | |
| | 1H), 7.51 (d, J = 5.1 Hz, 1H), 7.83 (dd, J = 8.1, | |
| | 1.5 Hz, 1H), 8.23 (dd, J = 4.8, 1.8 Hz, 1H), | |
| | 8.26 (br.s, 1H), 8.43-8.48 (m, 2H), 8.51 (s, 1H) | |
| 112 | (MeOD) δ 4.03 (s, 3H), 7.13 (dd, J = 7.3, 4.8 Hz, | 319.2 (M + 1) |
| | 1H), 7.19 (dd, J = 8.8, 1.8 Hz, 1H), 7.52 (d, J = 5.1 Hz, | |
| | 1H), 7.62 (d, J = 8.6 Hz, 1H), 7.89 (s, 1H), | |
| | 8.24 (dd, J = 4.8, 1.8 Hz, 1H), 8.35 (s, 1H), 8.45 (d, J = 5.1 Hz, | |
| | 1H), 8.52 (dd, J = 7.3, 1.8 Hz, 1H) | |
| 113 | (MeOD) δ 1.85 (dd, J = 13.5, 3.8 Hz, 1H), 1.92 (dd, | 362.3 (M + 1) |
| | J = 13.0, 4.1 Hz, 1H), 2.10 (brd, J = 13.5 Hz, 2H), | |
| | 2.87 (m, 1H), 3.11 (td, J = 13.0, 3.0 Hz, 2H), | |
| | 3.46 (brd, J = 12.1 Hz, 2H) 4.02 (s, 3H), 6.89 (d, J = 7.6 Hz, | |
| | 1H), 7.09 (dd, J = 7.6, 4.6 Hz, 1H), 7.25 (t, J = 7.8 Hz, | |
| | 1H), 7.48 (d, J = 5.1 Hz, 1H), 7.54 (d, J = 7.1 Hz, | |
| | 1H), 7.68 (brs, 1H), 8.24 (dd, J = 4.8, 1.8 Hz, | |
| | 1H), 8.40 (d, J = 5.1 Hz, 1H), 8.46 (dd, J = 7.6, | |
| | 2.0 Hz, 1H) | |
| 114 | (MeOD) δ 1.44 (s, 9H), 1.53-1.65 (m, 2H), | 462.3 (M + 1) |
| | 1.78-1.87 (m, 2H), 2.69 (m, 1H), 2.76-2.91 (m, 2H), | |
| | 4.03 (s, 3H), 4.14-4.24 (m, 2H), 6.85 (d, J = 7.1 Hz, 1H), | |
| | 7.08 (dd, J = 7.3, 4.8 Hz, 1H), 7.19 (t, J = 8.1 Hz, | |
| | 1H), 7.44 (d, J = 8.1 Hz, 1H), 7.48 (d, J = 5.1 Hz, | |
| | 1H), 7.72 (br.s, 1H), 8.23 (m, 1H), 8.39 (d, J = 5.1, | |
| | Hz, 1H), 8.50 (d, J = 7.6 Hz, 1H) | |
| 115 | (MeOD) δ 4.02 (s, 3H), 7.07 (dd, J = 7.6, 5.1 Hz, | 355.2 (M + 1) |
| | 1H), 7.22 (d, J = 7.6 Hz, 1H), 7.27-7.37 (m, 2H), | |
| | 7.39 (t, J = 8.0 Hz, 2H), 7.50 (d, J = 5.6 Hz, 1H), | |
| | 7.58-7.62 (m, 3H), 8.16 (br.s, 1H), 8.24 (dd, J = 5.1, | |
| | 2.0 Hz, 1H), 8.43 (d, J = 5.6 Hz, 1H), 8.51 (dd, J = 7.6, | |
| | 2.0 Hz, 1H) | |
| 116 | | 324.1 (M + 1) |
| 117 | (MeOD) δ 2.32 (s, 3H), 2.60 (m, 4H), 3.14 (m, 4H), | 377.3 (M + 1) |
| | 4.02 (s, 3H), 6.95 (d, J = 9.1 Hz, 2H), 7.08 (dd, J = 7.6, | |
| | 5.1 Hz, 1H), 7.41 (d, J = 5.6 Hz, 1H), 7.54 (d, J = 8.6 Hz, | |
| | 2H), 8.21 (dd, J = 5.1, 2.0 Hz, 1H), | |
| | 8.34 (d, J = 5.1 Hz, 1H), 8.43 (dd, J = 7.6, 2.0 Hz, 1H) | |
| 118 | (CDCl₃) δ 3.99 (s, 3H), 4.08 (s, 3H), 6.49 (d, J = 8.6 Hz, | 343.0 (M + 1) |
| | 1H), 7.01 (d, J = 8.1 Hz, 1H), 7.24 (t, J = 7.8 Hz, | |
| | 1H), 7.41 (d, J = 8.1 Hz, 1H), 7.59 (d, J = 5.6 Hz, | |
| | 1H), 7.93 (br.s, 1H), 8.37 (d, J = 5.6 Hz, 1H), | |
| | 8.49 (d, J = 8.6 Hz, 1H) | |
| 119 | (CDCl₃) δ 3.07 (s, 3H), 4.04 (s, 3H), 4.15 (s, 3H), | 387.0 (M + 1) |
| | 6.62 (dd, J = 8.6, 1.0 Hz, 1H), 7.59 (t, J = 7.8 Hz, | |
| | 1H), 7.69 (d, J = 8.1 Hz, 1H), 7.75 (d, J = 7.6 Hz, | |
| | 1H), 7.95 (br.s, 1H), 8.12 (br.s, 1H), 8.72 (d, J = 8.6 Hz, | |
| | 1H), 8.84 (s, 1H | |
| 120 | | 356.3 (M + 1) |
| 121 | | 398.3 (M + 1) |
| 144 | (CDCl₃) δ 1.79 (m, 2H), 2.16 (s, 3H), 3.42 (dd, J = 10.9, | 397.2 (M + 1) |
| | 6.3 Hz, 2H), 3.52 (dd, J = 12.1, 6.3 Hz, 2H), | |
| | 5.78 (m, 1H), 6.98 (ddd, J = 8.1, 2.2, 1.2 Hz, 1H), | |
| | 7.19 (d, J = 5.6 Hz, 1H), 7.23 (d, J = 8.3 Hz, 1H), | |
| | 7.53 (d, J = 5.4 Hz, 1H), 7.73 (ddd, J = 8.3, 2.2, 1.2 Hz, | |
| | 1H), 7.93 (m, 2H), 8.26 (s, 1H), 8.52 (d, J = 5.3 Hz, | |
| | 1H), 9.28 (m, 1H), 9.64 (s, 1H) | |

### Biological Data

### Compound IC50 Determination in Kinase Assays

Kinase assays were performed on recombinant purified enzymes using the non-radioactive IMAP fluorescence polarization assay format.

Compound stocks were prepared and diluted in DMSO and were added to kinase reactions at a 1/100 dilution for a final concentration of 1% DMSO. The components were added together to final concentrations listed in the tables below and incubated for two hours at room temperature.

Final Assay Conditions for CDK9/cyclinT1 Assay

| Reagant | Company |
|---|---|
| 1x Tween reaction buffer | Molecular Devices |
| 1 mM dithiothreitol | Fisher Biotech |
| 1% Dimethylsulfoxide | Fisher Scientific |
| 6 μM ATP | Cell Signaling Technology |
| 100 nM TAMRA-CDK7tide | Molecular Devices |
| 0.007 Units CDK9/cyclinT1 enzyme | Millipore |

Final Assay Conditions for CDK1/cyclinB Assay

| Reagant | Company |
|---|---|
| 1x Tween reaction buffer | Molecular Devices |
| 1 mM dithiothreitol | Fisher Biotech |
| 1% Dimethylsulfoxide | Fisher Scientific |
| 52 μM ATP | Cell Signaling Technology |
| 100 nM FAM-PKCepsilon peptide | Molecular Devices |
| 0.005 Units CDK1/cyclinB enzyme | Millipore |

Final Assay Conditions for CDK2/cyclinA Assay

| Reagant | Company |
|---|---|
| 1x Tween reaction buffer | Molecular Devices |
| 1 mM dithiothreitol | Fisher Biotech |
| 1% Dimethylsulfoxide | Fisher Scientific |
| 4.68 μM ATP | Cell Signaling Technology |
| 100 nM TAMRA-Histone H1 peptide | Molecular Devices |
| 0.009 Units CDK2/cyclinA enzyme | Millipore |

Final conditions for CDK7/cyclinH/Mat1 assay

| Reagant | Company |
|---|---|
| 1x Tween reaction buffer | Molecular Devices |
| 1 mM dithiothreitol | Fisher Biotech |
| 1% Dimethylsulfoxide | Fisher Scientific |
| 8.09 μM ATP | Cell Signaling Technology |
| 100 nM TAMRA-CDK7tide | Molecular Devices |
| 0.008 Units CDK7/cyclinH/MAT1 | Millipore |
| enzyme | |

The reactions were stopped by addition of stop solution.

CDK9/CDK1/CDK7: final stop concentration is 1× Developer buffer (85% A/15% B), IMAP beads 1/400

CDK2: final stop concentration is 1× Developer buffer (100% A), IMAP beads 1/400

Components of Stop Solution

| Reagant | Company |
|---|---|
| Developer Buffer A | Molecular Devices |
| Developer Buffer B | Molecular Devices |
| IMAP Beads/progressive | Molecular Devices |
| binding reagant | |

The samples were allowed to incubate for one more hour at room temperature and then the fluorescence polarization was measured. The plate normalized millipolarization (mP) values were plotted against the log of compound concentration for each compound and IC₅₀ values were calculated using the spotfire IC₅₀ calculator and confirmed by visual analysis.

The results of this assay as performed using the compounds of the invention are shown in Table F.

H5 (also referred as Ser2) Ab High Content Screen: RNA Polymerase II CTD Serine2 Phosphorylation Assay

1. Cells are plated into 384 well plates: 30 μl of Hep3B cells (4,000 cells/30 μl) in DMEM with 3% FBS are plated into wells of a tissue culture treated clear bottomed 384 well plate. Cells are allowed to adhere during an overnight incubation.

2. Cells are treated with compounds for two hours: 10 μl of DMEM with 3% FBS is added to each well with a final concentration of DMSO equal to 1%. Cells are incubated with compounds for 2 hours.

3. Cells are fixed and permeabilized. Cells are washed with 30 mM HEPES pH=7.3 then fixed in 4% paraformaldehyde in 30 mM HEPES pH=7.3 for one hour at room temperature. The fix is removed by washing the cells with Tris buffered saline (TBS) and the cells are permeabilized by incubation in TBS with 0.5% triton X-100 for 30 minutes at room temperature. Triton is removed by washing the cells with TBS.

4. Cells are stained with H5 monoclonal antibody and nuclei are stained with Hoechst dye 33342: Block cells by incubating in TBS with 1% Bovine serum albumin (BSA) for one hour at room temperature. Incubate primary H5 Ab (1/250 ) overnight at 4 degrees in TBS with 1% BSA and 0.1% Tween-20. The next day wash cells with TBS. Add secondary antibody, anti-IgM labeled with cy5 (1/450 ), and Hoecsht dye 33342 (10 μg/ml) in TBS with 1% BSA and incubate one hour at room temperature. Wash cells with PBS.

5. Measure staining using the In-cell Analyzer system from GE Healthcare. Nuclei were located using the Hoecsht 33342 dye signal and H5 Antibody staining was quantified by measuring cy5 label. Nuclear intensity of H5 staining was plotted against the log of compound concentration for each compound and IC-50 values were calculated using the spotfire IC-50 calculator and confirmed by visual analysis.

The results of this assay as performed using a selection of the compounds of the invention are shown in Table F.

TABLE F

| Com- | CDK1 | CDK2 | CDK4 | CDK5 | CDK7 | CDK9 | Ser2 |
|---|---|---|---|---|---|---|---|
| pound | IC₅₀ | IC₅₀ | IC₅₀ | IC₅₀ | IC₅₀ | IC₅₀ | IC₅₀ |
| no. | (μM) | (μM) | (μM) | (μM) | (μM) | (μM) | (μM) |
| 1 | * | * | | | | * | |
| 2 | **** | **** | | | | * | **** |
| 3 | * | * | * | * | * | * | * |
| 4 | * | | | | | * | |
| 5 | * | | | | | * | |
| 6 | ** | * | ** | | * | * | ** |
| 7 | * | | | | | * | |
| 8 | *** | | | | | ** | |
| 9 | * | | | | | * | |
| 10 | ** | | | | | * | |
| 11 | *** | | | | | **** | |
| 13 | **** | | | | | **** | |
| 15 | **** | | | | | * | |
| 17 | * | | | | | * | |
| 18 | * | * | | | | * | |
| 19 | ** | | | | | * | |
| 20 | ** | | | | | * | |
| 21 | * | * | | | | * | ** |
| 22 | ** | * | | | | * | ** |
| 23 | ** | * | *** | * | | * | ** |
| 24 | ** | * | | ** | | * | *** |
| 25 | * | * | ** | * | * | * | * |
| 27 | ** | | | | | * | |
| 28 | *** | | | | | * | |
| 31 | * | | | | | * | |
| 32 | * | * | *** | * | * | * | * |
| 33 | **** | | | | | *** | |
| 34 | * | | | | | * | |
| 35 | * | | | | | * | |
| 36 | * | | | | | * | |
| 38 | ** | | | | | * | |
| 39 | * | | | | | * | |
| 40 | *** | | | | | * | |
| 41 | * | | | | | * | |
| 42 | * | * | | | | * | * |
| 43 | * | | | | | * | |
| 44 | * | * | *** | * | | * | |
| 45 | * | * | ** | * | **** | * | * |
| 46 | * | * | ** | * | * | * | * |
| 47 | ** | * | * | * | | * | |
| 48 | * | | ** | * | | * | * |
| 50 | **** | **** | | | | **** | |
| 51 | * | | | | | * | |
| 52 | * | * | * | * | * | * | * |
| 53 | *** | ** | | | | * | |
| 54 | * | * | | | | * | |
| 55 | * | * | * | * | * | * | * |
| 56 | * | * | | | | * | |
| 57 | **** | **** | | | | * | |
| 58 | * | * | * | * | | * | * |
| 59 | * | * | ** | * | | * | *** |
| 60 | * | * | * | * | * | * | * |
| 61 | * | * | * | *** | * | * | * |
| 62 | ** | ** | | | | * | |
| 63 | * | * | ** | * | | * | |
| 64 | * | * | ** | * | | * | |
| 65 | * | * | *** | * | | * | * |
| 66 | **** | *** | | | | * | |
| 67 | **** | ** | *** | ** | | * | |
| 68 | * | * | *** | * | | * | |
| 69 | * | * | *** | * | | * | |
| 70 | ** | ** | | | | * | |
| 71 | ** | **** | | | | * | |
| 72 | * | * | *** | * | | * | * |
| 73 | * | * | *** | ** | | * | ** |
| 74 | * | * | *** | * | * | * | * |
| 75 | ** | * | | | | * | |
| 76 | ** | * | | | | * | |
| 77 | * | * | | | | * | |
| 78 | ** | * | | | | * | |
| 79 | * | * | *** | * | | * | |
| 80 | * | * | ** | * | * | * | * |
| 81 | * | * | | | | * | |
| 82 | ** | *** | | | | * | |
| 83 | * | * | *** | * | | * | |
| 84 | * | * | * | * | | * | * |
| 85 | * | * | | | | * | |
| 86 | **** | | | | | *** | |
| 87 | *** | | | | | *** | |
| 88 | *** | | | | | ** | |
| 89 | **** | **** | | | | **** | |
| 90 | **** | **** | | | | **** | |
| 91 | * | * | *** | * | **** | * | **** |
| 92 | **** | **** | | | | * | |
| 93 | * | * | *** | * | | * | * |
| 94 | *** | * | | | | *** | |
| 95 | * | * | | | | * | |
| 96 | * | * | | | | * | |
| 97 | ** | ** | | | | * | |
| 98 | * | * | | | | * | |
| 99 | * | * | * | * | ** | * | * |
| 100 | * | * | | | | * | |
| 101 | **** | * | | | | * | |
| 102 | * | * | | | | * | *** |
| 103 | * | * | | | | * | |
| 104 | **** | **** | | | | **** | |
| 105 | **** | **** | *** | ** | | **** | |
| 106 | ** | *** | | | | * | |
| 107 | ** | * | | | | * | |
| 108 | * | * | | | | * | |
| 109 | **** | * | | | | * | |
| 110 | * | * | | | | * | * |
| 111 | ** | * | | | | * | |
| 112 | * | * | | | | * | * |
| 113 | * | * | | | | * | |
| 114 | **** | **** | | | | **** | |
| 115 | **** | **** | | | | * | |
| 116 | **** | **** | | | | **** | |
| 117 | ** | * | * | * | | * | |
| 118 | **** | | | | | ** | |
| 119 | * | * | *** | ** | **** | * | **** |
| 120 | * | *** | *** | * | | * | |
| 121 | ** | *** | *** | *** | | * | |

IC₅₀ Key
* < 5 μM
5 μM < ** < 15 μM
15 μM < *** < 50 μM
50 μM < *****

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

### INCORPORATION BY REFERENCE

The entire contents of all patents, published patent applications and other references cited herein are hereby expressly incorporated herein in their entireties by reference.

1. A compound of the Formula I: 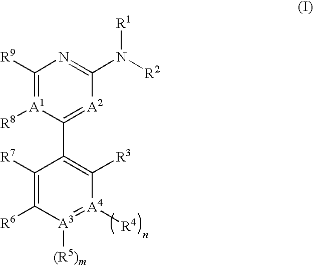
and pharmaceutically acceptable salts, enantiomers, stereoisomers, rotamers, tautomers, diastereomers, or racemates thereof;
wherein
m is 0 or 1;
n is 0 or 1;
A¹, A², A³ and A⁴ are each, independently, C, C(H) or N;
R¹, R², R³, R⁴, R⁵, R⁶, R⁷, R⁸ and R⁹ are each, independently, selected from the group consisting of hydrogen, halogen, substituted or unsubstituted amino, substituted or unsubstituted C₁₋₂₀-alkyl, substituted or unsubstituted C₁₋₂₀-alkoxy, substituted or unsubstituted aryl, and substituted or unsubstituted C₃₋₇-cycloalkyl;
or R⁴ or R⁵ may independently be O⁻;
or R³ and R⁴ can also together form a six-membered ring of the following structure: 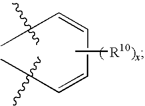
wherein x is 0, 1 or 2, and R¹⁰ is selected from the group consisting of hydrogen, halogen, substituted or unsubstituted amino, substituted or unsubstituted C₁₋₆-alkyl, substituted or unsubstituted C₁₋₆-alkoxy, substituted or unsubstituted aryl, and substituted or unsubstituted C₃₋₇-cycloalkyl;
or each of R⁷ and R⁸ can independently represent CH₂ or CH₂CH₂ groups that are bonded to one another, such that a six- or seven-membered ring is formed, or each of R³ and R⁸ can independently represent CH₂ or CH₂CH₂ groups that are bonded to one another, such that a six- or seven-membered ring is formed, wherein the six- or seven-membered rings may be independently substituted one or more times with hydrogen, halogen, substituted or unsubstituted amino, substituted or unsubstituted C₁₋₆-alkyl, substituted or unsubstituted C₁₋₆-alkoxy, substituted or unsubstituted aryl, or substituted or unsubstituted C₃₋₇-cycloalkyl.

2. The compound of claim 1, wherein R⁴ is hydrogen, (CH₂)₃₋₂₀CH₃ or (CH₂)₃₋₂₀NH₂, wherein the CH₂ groups can be independently interrupted one or more times with N(H), N(CH₃), O, or C(O).

3. The compound of claim 1, wherein the C₁₋₂₀-alkyl and C₁₋₂₀-alkoxy groups are C₁₋₆-alkyl and C₁₋₆-alkoxy groups.

4. The compound of claim 1, wherein at least one of R³, R⁷ and R⁸ is not H.

5. The compound of claim 1, wherein m is 0, A⁴ is C and A³ is N.

6. The compound of claim 1, wherein A³ is C, A⁴ is N and n is 0.

7. The compound of claim 1, wherein m and n are 0, A¹ is C and A³ and A⁴ are N.

8. The compound of claim 1, wherein A¹ and A² are N.

9. The compound of claim 1, wherein n is 0, A¹ is C, and A³ and A⁴ are N.

10. The compound of claim 1, wherein
R¹ is selected from the group consisting of hydrogen and C₁₋₆-alkyl;
R² is selected from the group consisting of substituted-aryl and substituted-C₁₋₆-alkyl;
R³ and R⁴ are each, independently, selected from the group consisting of hydrogen, halogen, C₁₋₆-alkyl, C₁₋₆alkoxy, substituted or unsubstituted amino, substituted or unsubstituted aryl, and substituted or unsubstituted C₃₋₇-cycloalkyl;
R⁶ is selected from the group consisting of hydrogen, halogen and C₁₋₆-alkyl; and
R⁷, R⁸ and R⁹ are each, independently, selected from the group consisting of hydrogen and C₁₋₆-alkyl;
or R³ and R⁴ can also together form a six-membered ring of the following structure: 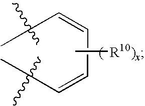
wherein x is 0, 1 or 2, and R¹⁰ is selected from the group consisting of hydrogen, halogen, substituted or unsubstituted amino, substituted or unsubstituted C₁₋₆-alkyl, substituted or unsubstituted C₁₋₆-alkoxy, and substituted or unsubstituted C₃₋₇-cycloalkyl;
or each of R⁷ and R⁸ can independently represent CH₂ or CH₂CH₂ groups that are bonded to one another, such that a six- or seven-membered ring is formed, or each of R³ and R⁸ can independently represent CH₂ or CH₂CH₂ groups that are bonded to one another, such that a six- or seven-membered ring is formed, wherein the six- or seven-membered rings may be independently substituted one or more times with hydrogen, halogen, substituted or unsubstituted amino, substituted or unsubstituted C₁₋₆-alkyl, substituted or unsubstituted C₁₋₆-alkoxy, substituted or unsubstituted aryl, or substituted or unsubstituted C₃₋₇-cycloalkyl.

11. The compound of claim 1, wherein R¹ is H.

12. The compound of claim 1, wherein R² is aryl, which is independently substituted one or more times with substituted or unsubstituted alkyl, hydroxy, ether, thioether, amino, aryl, heterocycle, electron-withdrawing group or electron-withdrawing atom.

13. The compound of claim 1, wherein R² is benzothiazole, benzimidazole, benzoxazole, indazole or phenyl, all of which may be independently substituted one or more times with halogen, nitro, hydroxy, nitrile, substituted or unsubstituted amino, ether, ester, carboxylic acid, substituted or unsubstituted amide, substituted or unsubstituted sulfone, substituted or unsubstituted sulfonamide, substituted or unsubstituted phenyl or substituted or unsubstituted heterocycle.

14. A compound of the Formula II: 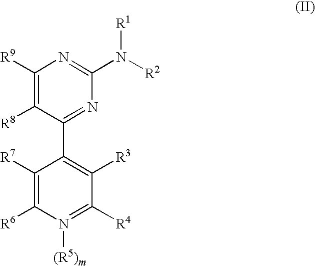
and pharmaceutically acceptable salts, enantiomers, stereoisomers, rotamers, tautomers, diastereomers, or racemates thereof;
wherein
m is 0 or 1;
R¹, R², R³, R⁴, R⁵, R⁶, R⁷, R⁸ and R⁹ are each, independently, selected from the group consisting of hydrogen, halogen, substituted or unsubstituted amino, substituted or unsubstituted C₁₋₂₀-alkyl, substituted or unsubstituted C₁₋₂₀-alkoxy, substituted or unsubstituted aryl, and substituted or unsubstituted C₃₋₇-cycloalkyl;
or R⁵ may be O⁻;
or R³ and R⁴ can also together form a six-membered ring of the following structure: 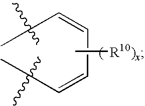
wherein x is 0, 1 or 2, and R¹⁰ is selected from the group consisting of hydrogen, halogen, substituted or unsubstituted amino, substituted or unsubstituted C₁₋₆-alkyl, substituted or unsubstituted C₁₋₆-alkoxy, and substituted or unsubstituted C₃₋₇-cycloalkyl;
or each of R⁷ and R⁸ can independently represent CH₂ or CH₂CH₂ groups that are bonded to one another, such that a six- or seven-membered ring is formed, or each of R³ and R⁸ can independently represent CH₂ or CH₂CH₂ groups that are bonded to one another, such that a six- or seven-membered ring is formed, wherein the six- or seven-membered rings may be independently substituted one or more times with hydrogen, halogen, substituted or unsubstituted amino, substituted or unsubstituted C₁₋₆-alkyl, substituted or unsubstituted C₁₋₆-alkoxy, substituted or unsubstituted aryl, or substituted or unsubstituted C₃₋₇-cycloalkyl.

15. The compound of claim 14, wherein R⁴ is hydrogen, (CH₂)₃₋₂₀CH₃ or (CH₂)₃₋₂₀NH₂, wherein the CH₂ groups can be independently interrupted one or more times with N(H), N(CH₃), O, or C(O).

16. The compound of claim 14, wherein the C₁₋₂₀-alkyl and C₁₋₂₀-alkoxy groups are C₁₋₆-alkyl and C₁₋₆-alkoxy groups.

17. The compound of claim 14, wherein at least one of R³, R⁷ and R⁸ is not H.

18. The compound of claim 14, wherein
m is 0;
R¹ is selected from the group consisting of hydrogen and C₁₋₆-alkyl;
R² is selected from the group consisting of substituted-aryl and substituted-C₁₋₆-alkyl;
R³ and R⁴ are each, independently, selected from the group consisting of hydrogen, halogen, C₁₋₆-alkyl, C₁₋₆-alkoxy, substituted or unsubstituted amino, substituted or unsubstituted aryl, and substituted or unsubstituted C₃₋₇-cycloalkyl;
R⁶ is selected from the group consisting of hydrogen, halogen and C₁₋₆-alkyl; and
R⁷, R⁸ and R⁹ are each, independently, selected from the group consisting of hydrogen, halogen, C₁₋₆-alkyl, C₁₋₆-alkoxy, and substituted or unsubstituted amino.

19. The compound of claim 14, wherein R² is aryl, which is independently substituted one or more times with substituted or unsubstituted alkyl, hydroxy, ether, thioether, amino, substituted or unsubstituted aryl, heterocycle, electron-withdrawing group or electron-withdrawing atom.

20. The compound of claim 14, wherein R² is benzothiazole, benzimidazole, benzoxazole, indazole or phenyl, all of which may be independently substituted one or more times with halogen, nitro, hydroxy, nitrile, substituted or unsubstituted amino, ether, ester, carboxylic acid, substituted or unsubstituted amide, substituted or unsubstituted sulfone, substituted or unsubstituted sulfonamide, substituted or unsubstituted phenyl or substituted or unsubstituted heterocycle.

21. The compound of claim 14, wherein R³ is selected from the group consisting of C₁₋₆-alkyl, C₁₋₆-alkoxy, and substituted or unsubstituted amino.

22. The compound of claim 14, wherein
m is 0;
R¹ is selected from the group consisting of hydrogen and C₁₋₆-alkyl;
R² is benzothiazole, benzimidazole, benzoxazole, indazole or phenyl, all of which may be independently substituted one or more times with nitrile, halogen, nitro, hydroxy, substituted or unsubstituted amino, ether, ester, carboxylic acid, substituted or unsubstituted amide, substituted or unsubstituted sulfone, substituted or unsubstituted sulfonamide, substituted or unsubstituted phenyl or substituted or unsubstituted heterocycle;
R³ and R⁴ are each, independently, selected from the group consisting of hydrogen, halogen, C₁₋₆-alkyl, C₁₋₆-alkoxy, substituted or unsubstituted amino, substituted or unsubstituted aryl, and substituted or unsubstituted C₃₋₇-cycloalkyl;
R⁶ and R⁹ are H; and
R⁷, and R⁸ are each, independently, selected from the group consisting of hydrogen, C₁₋₆-alkyl and C₁₋₆-alkoxy.

23. The compound of claim 14, wherein
R² is selected from the group consisting of aryl substituted halogen, nitro, hydroxyl, nitrile, ether, ester, substituted or unsubstituted phenyl, substituted or unsubstituted heterocycle, SO₂(R¹¹) or N(R¹²)R¹³, and (CH₂)₁₋₆N(R¹²)R¹³;
R³ is selected from the group consisting of C₁₋₆-alkyl, C₁₋₆-alkoxy and N(R¹²)R¹³; and
R⁴ is selected from the group consisting of hydrogen and N(R¹²)R¹³;
wherein R¹¹ is selected from the group consisting of H, C₁₋₆-alkyl and NH₂;
wherein R¹² and R¹³ are each, independently, selected from the group consisting of H and (C₁₋₆-alkyl)₀₋₁G, wherein G is selected from the group consisting of H, COOH, NH₂, N(H)C(O)C₁₋₆-alkyl, N(C₁₋₆-alkyl)C(O)C₁₋₆-alkyl, N(H)C₁₋₆-alkyl, OH, OC(O)C₁₋₆-alkyl, C₃₋₇-cycloalkyl, phenyl, substituted phenyl, C(O)OC₁-C₆-alkyl, C(O)C₁₋₆-alkyl-COOH, C(O)C₁-C₄-alkyl, C(O)-aryl, morpholino, imidazole, pyrrolidin-2-one, substituted or unsubstituted heterocycle, pyrazole, pyridine, oxazole, thiazole, isoxazole, triazole, tetrazole, pyrimidine, pyridazine, pyrazine, piperazine and piperidine.

24. The compound of claim 14, wherein R² is substituted or unsubstituted imidazole, oxazole or triazole.

25. The compound of claim 14, wherein
m is 0;
R¹ is hydrogen;
R² is selected from the group consisting of aryl substituted halogen, nitro, hydroxyl, nitrile, ether, ester, substituted or unsubstituted phenyl, substituted or unsubstituted heterocycle, SO₂(R¹¹) or N(R¹²)R¹³, and (CH₂)₁₋₆N(R¹²)R¹³;
R³ and R⁴ are each, independently, selected from the group consisting of hydrogen, halogen, C₁₋₆-alkyl, C₁₋₆-alkoxy, substituted or unsubstituted amino, substituted or unsubstituted aryl, and substituted or unsubstituted C₃₋₇-cycloalkyl;
R⁶ is selected from the group consisting of hydrogen, chloro, and CH₃; and
R⁷ and R⁸ are each, independents, selected from the group consisting of hydrogen, halogen, substituted or unsubstituted amino, C₁₋₆-alkyl and C₁₋₆-alkoxy;
R⁹ is hydrogen;
wherein R¹¹ is selected from the group consisting of H, C₁₋₆-alkyl and NH₂;
wherein R¹² and R¹³ are each, independently, selected from the group consisting of H and (C₁₋₆alkyl)₀₋₁G, wherein G is selected from the group consisting of H, COOH, NH₂, N(H)C(O)C, alkyl, N(C₁₋₆-alkyl)C(O)C, alkyl, N(H)C₁₋₆alkyl, OH, OC(O)C₁₋₆alkyl, C₃₋₇-cycloalkyl, phenyl, substituted phenyl, C(O)OC₁-C₆-alkyl, C(O)C₁₋₆alkyl-COOH, C(O)C₁-C₄-alkyl, C(O)-aryl, morpholino, imidazole and pyrrolidin-2-one.

26. The compound of claim 14, wherein
m is 0;
R¹ is hydrogen;
R² is selected from the group consisting of aryl substituted with halogen, nitro, hydroxyl, nitrile, ether, ester, substituted or unsubstituted phenyl, substituted or unsubstituted heterocycle, SO₂(R¹¹), N(R¹²)R¹³, or (CH₂)₁₋₆N(R¹²)R¹³;
R³ is selected from the group consisting of halogen, CH₃, CH₂CH₃, OCH₃ and substituted or unsubstituted amine;
R⁴ is selected from the group consisting of hydrogen, halogen, CH₃, OCH₃, N(H)(CH₂)₃NH₂, N(H)(CH₂)₃N(H)C(O)CH₃, N(CH₃)(CH₂)₃N(H)CH₃, N(CH₃)(CH₂)₃N(CH₃)C(O)CH₃, N(H)(CH₂)₃OH, N(H)(CH₂)₃OC(O)CH₃, N(H)(CH₂)₃N(H)-Ph-CH₂NH₂, N(H)(CH₂)₃N(H)-Ph-CH₂N(H)C(O)CH₃, N(H)(CH₂)₃CO₂H, N(H)CH₂Ph, N(H)(CH₂)₃OCH₃, N(H)(CH₂)₃-pyrrolidin-2-one, N(H)(CH₂)₃—N-morpholino, N(H)(CH₂)₃-imidazole, N(H)(CH₂)₃—N(H)CH₃;
R⁶ is selected from the group consisting of hydrogen, chloro, and CH₃; and
R⁷ and R⁸ are each, independently, selected from the group consisting of hydrogen, halogen, substituted or unsubstituted amino, C₁₋₆-alkyl and C₁₋₆-alkoxy;
R⁹ is hydrogen;
wherein R¹¹ is selected from the group consisting of H, C₁₋₆-alkyl and NH₂;
wherein R¹² and R¹³ are each, independents, selected from the group consisting of H and (C₁₋₆alkyl)₀₋₁G, wherein G is selected from the group consisting of H, COOH, NH₂, N(H)C(O)C₁₋₆alkyl, N(C₁₋₆alkyl)C(O)C₁₋₆alkyl, N(H)C₁₋₆alkyl, OH, OC(O)C₁₋₆alkyl, C₃₋₇-cycloalkyl, phenyl, substituted phenyl, C(O)OC₁-C₆-alkyl, C(O)C₁₋₆alkyl-COOH, C(O)C₁-C₄-alkyl, C(O)-aryl, morpholino, imidazole and pyrrolidin-2-one.

27. The method of claim 26, wherein R³ is selected from the group consisting of CH₃, CH₂CH₃, OCH₃, OCH₂CH₃, O(CH₂)₃NH₂, O(CH₂)₃N(H)C(O)CH₃, N(H)(CH₂)₃OH, N(H)(CH₂)₃OCH₃, N(H)(CH₂)₃OC(O)CH₃, F, Cl, Br, NH₂, N(H)C(O)CH₃, N(H)(CH₂)₃CH₃, N(H)(CH₂)₂NH₂, N(H)(CH₂)₂N(H)C(O)CH₃, N(H)(CH₂)₂N(H)CH₃, N(H)(CH₂)₂N(CH₃)₂, N(H)CH₂C(CH₃)₂CH₂N(H)C(O)CH₃, N(H)(CH₂)₂-imidazole, N(H)(CH₂)₂-pyrazole, N(H)(CH₂)₃-imidazole, N(H)(CH₂)₃-pyrazole, N(H)(CH₂)₃-imidazole-CH₃, N(H)(CH₂)₃-pyrazole-CH₃, N(H)(CH₂)₃-imidazole-(CH₃)₂, N(H)(CH₂)₃-pyrazol(CH₃)₂, N(H)(CH₂)₃-morpholino, N(H)(CH₂)₃-piperidine, N(H)(CH₂)₃-piperazine, N(H)(CH₂)₃-lactam, N(H)(CH₂)₃-pyrrolidin-2-one, N(H)(CH₂)₃NH₂, N(H)(CH₂)₃N(H)C(O)CH₃, N(H)(CH₂)₃N(H)C(O)C(CH₃)₃, N(H)(CH₂)₃N(H)C(O)CH₂C(CH₃)₃, N(H)CH₂C(CH₃)₂CH₂NH₂, N(H)(CH₂)₃N(H)CH₃, N(H)(CH₂)₃N(CH₃)₂, N(H)(CH₂)₃N(H)C(H)(CH₃)₂, N(H)(CH₂)₃N(H)S(O)₂CH₃, N(H)(CH₂)₄NH₂, N(H)(CH₂)₄N(H)C(O)CH₃, and N(H)-Ph-S(O)₂CH₃.

28. A compound of the Formula III: 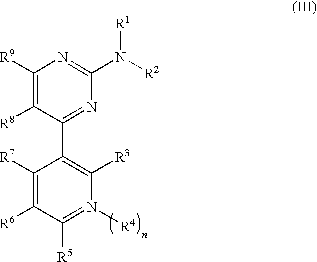
and pharmaceutically acceptable salts, enantiomers, stereoisomers, rotamers, tautomers, diastereomers, or racemates thereof;
wherein
n is 0 or 1;
R¹, R², R³, R⁴, R⁵, R⁶, R⁷, R⁸ and R⁹ are each, independently, selected from the group consisting of hydrogen, halogen, substituted or unsubstituted amino, substituted or unsubstituted C₁₋₂₀-alkyl, substituted or unsubstituted C₁₋₂₀-alkoxy, substituted or unsubstituted aryl, and substituted or unsubstituted C₃₋₇-cycloalkyl;
or R⁴ may be O⁻;
or R³ and R⁴ can also together form a six-membered ring of the following structure: 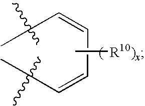
wherein x is 0, 1 or 2, and R¹⁰ is selected from the group consisting of hydrogen, halogen, substituted or unsubstituted amino, substituted or unsubstituted C₁₋₆-alkyl, substituted or unsubstituted C₁₋₆-alkoxy, and substituted or unsubstituted C₃₋₇-cycloalkyl;
or each of R⁷ and R⁸ can independently represent CH₂ or CH₂CH₂ groups that are bonded to one another, such that a six- or seven membered ring is formed, or each of R³ and R⁸ can independently represent CH₂ or CH₂CH₂ groups that are bonded to one another, such that a six- or seven-membered ring is formed, wherein the six or seven-membered rings may be independently substituted one or more times with hydrogen, halogen, substituted or unsubstituted amino, substituted or unsubstituted C₁₋₆-alkyl, substituted or unsubstituted C₁₋₆-alkoxy, substituted or unsubstituted aryl, or substituted or unsubstituted C₁₋₇cycloalkyl.

29. The compound of claim 28, wherein R³ is halogen, (CH₂)₃₋₂₀CH₃ or (CH₂)₃₋₂₀NH₂, wherein the CH₂ groups can be independently interrupted one or more times with N(H), N(CH₃), O, or C(O).

30. The compound of claim 28, wherein the C₁₋₂₀-alkyl and C₁₋₂₀-alkoxy groups are C₁₋₆-alkyl and C₁₋₆-alkoxy groups.

31. The compound of claim 28, wherein at least one of R³, R⁷ and R⁸ is not H.

32. The compound of claim 28, wherein R¹ is H.

33. The compound of claim 28, wherein n is 0, and R⁶, R⁷ and R⁹ are hydrogen.

34. The compound of claim 28, wherein R² is benzothiazole, benzimidazole, benzoxazole, indazole or phenyl, all of which may be independently substituted one or more times with halogen, nitro, hydroxy, nitrile, substituted or unsubstituted amino, ether, ester, carboxylic acid, substituted or unsubstituted amide, substituted or unsubstituted sulfone, substituted or unsubstituted sulfonamide, substituted or unsubstituted phenyl or substituted or unsubstituted heterocycle.

35. The compound of claim 28, wherein
n is 0;
R¹ is selected from the group consisting of hydrogen and C₁₋₆-alkyl;
R² is selected from the group consisting of aryl substituted with halogen, nitro, hydroxyl, nitrile, ether, ester, substituted or unsubstituted phenyl, substituted or unsubstituted heterocycle, SO₂(R¹¹), N(R¹²)R¹³, or (CH₂)₁₋₆N(R¹²)R¹³;
R³ is selected from the group consisting of hydrogen, halogen, C₁₋₆-alkyl, C₁₋₆-alkoxy, substituted or unsubstituted amino, substituted or unsubstituted aryl, and substituted or unsubstituted C₃₋₇-Cycloalkyl;
R⁷ and R⁸ are hydrogen, halogen, alkyl, alkoxy, or substituted or unsubstituted amino;
R⁶ and R⁹ are H,
wherein R¹¹ is selected from the group consisting of H, C₁₋₆-alkyl and NH₂;
wherein R¹² and R¹³ are each, independently, selected from the group consisting of H and (C₁₋₆-alkyl)₀₋₁G, wherein G is selected from the group consisting of H, COOH, NH₂, N(H)C(O)C₁₋₆alkyl, N(C₁₋₆alkyl)C(O)C₁₁alkyl, N(H)C, alkyl, OH, OC(O)C₁₋₆alkyl, C₃₋₇-cycloalkyl, phenyl, substituted phenyl, C(O)OC₁-C₆-alkyl, C(O)C₁₋₆alkyl-COOH, C(O)C₁-C₄-alkyl, C(O)aryl, morpholino, imidazole, pyrrolidin-2-one substituted or unsubstituted heterocycle, pyrazole, pyridine, oxazole, thiazole, isoxazole, triazole, tetrazole, pyrimidine, pyridazine, pyrazine, piperazine and piperidine.

36. The compound of claim 28, wherein
R³ is selected from the group consisting of halogen, C₁₋₆-alkyl, C₁₋₆alkoxy, and N(R¹²)R¹³;
wherein R¹² and R¹³ are each, independently, selected from the group consisting of H and (C₁₋₆-alkyl)₀₋₁G, wherein G is selected from the group consisting of H, COOH, NH₂, N(H)C(O)C₁₋₆alkyl, N(C C₁₋₆alkyl)C(O)C₁₋₆-alkyl, N(H)C₁₋₆-alkyl, OH, OC(O)C, alkyl, C₃₋₇-cycloalkyl, phenyl, substituted phenyl, C(O)OC₁-C₆-alkyl, C(O)C₁₋₆alkyl-COOH, C(O)C₁-C₄-alkyl, C(O)-aryl, morpholino, imidazole, pyrrolidin-2-one, substituted or unsubstituted heterocycle, pyrazole, pyridine, oxazole, thiazole, isoxazole, triazole, tetrazole, pyrimidine, pyridazine, pyrazine, piperazine and piperidine.

37. The compound of claim 28, wherein
R¹ is hydrogen;
R² is hydrogen, CH₃, C₃₋₇-cyclohexyl, indazole, benzothiazole, benzimidazole, benzoxazole or phenyl, wherein the cyclohexyl, indazole, benzothiazole, benzimidazole, benzoxazole or phenyl groups are independently substituted one or more times with chloro, S(O)₂CH₃, C(O)NH₂, C(O)O-t-buyl, piperidine, piperidine substituted by t-butyl, oxazole, N(H)C(O)CH₃, 1,2,4-triazole substituted by methyl, SO₂NH₂, cyano, C(O)OEt, phenyl, S(O)₂N(H)CH₃, S(O)₂N(Et)₂, S(O)₂N(H)Et, S(O)₂N-imidazole, S(O)₂N(H)butyl, pyrrolidine, S(O)₂N(H)cyclohexyl, COOH, C(O)N(H)(CH₂)₂N(H)C(O)CH₃, C(O)N(H)(CH₂)₂N(CH₃)₂, C(O)N(H)Et, NO₂ or S(O)₂N(H)(CH₂)₂OH.

38. The compound of claim 28, wherein
R³ is selected from the group consisting of CH₃, CH₂CH₃, OCH₃, OCH₂CH₃, O(CH₂)₃NH₂, O(CH₂)₃N(H)C(O)CH₃, N(H)(CH₂)₃OH, N(H)(CH₂)₃OCH₃, N(H)(CH₂)₃OC(O)CH₃, F, Cl, Br, NH₂, N(H)C(O)CH₃, N(H)(CH₂)₃CH₃, N(H)(CH₂)₂NH₂, N(H)(CH₂)₂N(H)C(O)CH₃, N(H)(CH₂)₂N(H)CH₃, N(H)(CH₂)₂N(CH₃)₂, N(H)CH₂C(CH₃)₂CH₂N(H)C(O)CH₃, N(H)(CH₂)₃-imidazole, N(H)(CH₂)₂-pyrazole, N(H)(CH₂)₃-imidazole, N(H)(CH₂)₃-pyrazole, N(H)(CH₂)₃-imidazole-CH₃, N(H)(CH₂)₃-pyrazole-CH₃, N(H)(CH₂)₃-imidazole-(CH₃)₂, N(H)(CH₂)₃-pyrazole-(CH₃)₂, N(H)(CH₂)₃-morpholino, N(H)(CH₂)₃-piperidine, N(H)(CH₂)₃-piperazine, N(H)(CH₂)₃-lactam, N(H)(CH₂)₃-pyrrolidin-2-one, N(H)(CH₂)₃NH₂, N(H)(CH₂)₃N(H)C(O)CH₃, N(H)(CH₂)₃N(H)C(O)C(CH₃)₃, N(H)(CH₂)₃N(H)C(O)CH₂C(CH₃)₃, N(H)CH₂C(CH₃)₂CH₂NH₂, N(H)(CH₂)₃N(H)CH₃, N(H)(CH₂)₃N(CH₃)₂, N(H)(CH₂)₃N(H)C(H)(CH₃)₂, N(H)(CH₂)₃N(H)S(O)₂CH₃, N(H)(CH₂)₄NH₂, N(H)(CH₂)₄N(H)C(O)CH₃, and N(H>Ph-S(O)₂CH₃.

39. The compound of claim 28, wherein
R⁵ is selected from the group consisting of fluorine, hydrogen and OCH₃.

40. The compound of claim 28, wherein
R⁸ is selected from the group consisting of hydrogen, fluorine, OCH₃ and CH₃.

41. The compound of claim 1, wherein the compound of Formula I is represented by a compound selected from Table A, Table B, or Table C.

42.-47. (canceled)

48. A method of treating a protein kinase-associated disorder comprising administering to a subject in need thereof a pharmaceutically acceptable amount of a compound such that the protein kinase-associated disorder is treated, wherein the compound is a compound of the Formula I, Formula II or Formula III.

49. The method of claim 48, wherein the compound is selected from the group consisting of compounds listed in Table A, Table B, or Table C.

50. The method of claim 48, wherein the protein kinase is selected from the group consisting of CDK1, CDK2, CDK3, CDK4, CDK5, CDK6, CDK7, CDK8 and CDK9.

51. The method of claim 48, wherein the protein kinase-associated disorder is cancer.

52.-57. (canceled)

58. A method of treating cancer comprising administering to a subject in need thereof a pharmaceutically acceptable amount of a compound such that the cancer is treated, wherein the compound is a compound of the Formula I, Formula II or Formula III.

59.-70. (canceled)
